(19) United States
(12) Patent Application Publication
(10) Pub. No.: US 2025/0169509 A1
(43) Pub. Date: May 29, 2025

(54) ENDOPHYTE COMPOSITIONS AND METHODS FOR IMPROVED PLANT HEALTH

(71) Applicant: INDIGO AG, INC., Charlestown, MA (US)

(72) Inventors: Victoria KNIGHT-CONNONI, Holliston, MA (US); Scott SCHAEFFER, Malden, MA (US); Rebecca RYAN, Swampscott, MA (US); Zhanshan DONG, Johnston, IA (US)

(21) Appl. No.: 18/843,000

(22) PCT Filed: Mar. 3, 2023
(86) PCT No.: PCT/US2023/063741

§ 371 Date: Aug. 30, 2024

Related U.S. Application Data
(60) Provisional application No. 63/316,100, filed on Mar. 3, 2022.

(51) Int. Cl. A01N 65/00; A01N 63/22; A01N 65/44; A01P 21/00; C12N 1/16; C12N 1/20; C12R 1/07; C12R 1/645

(52) U.S. Cl. CPC A01N 65/00; A01N 63/22; A01N 65/44; A01P 21/00; C12N 1/165; C12N 1/205; C12R 2001/07; C12R 2001/645

(57) ABSTRACT

This invention relates to compositions and methods for improving plant health, wherein a plant is heterologously disposed to one or more endophytes, or derived from a plant element heterologously disposed to one or more endophytes.

### CROSS REFERENCE TO RELATED APPLICATIONS

This application claims the benefit of U.S. Provisional Application No. 63/316,100, filed Mar. 3, 2022, which is hereby incorporated by reference in its entirety.

### SEQUENCE LISTING

The instant application contains a Sequence Listing with 39 sequences which has been submitted via the USPTO Patent Center and is hereby incorporated by reference in its entirety. Said XML copy, created on Mar. 3, 2023, is named 10245PCT1 Sequence Listing.xml, and is 42 Kilobytes in size.

### BACKGROUND

According to the United Nations Food and Agriculture Organization, the world's population will exceed 9.6 billion people by the year 2050, which will require significant improvements in agriculture to meet growing food demands. There is a need for improved agricultural plants that will enable a near doubling of food production with fewer resources and more environmentally sustainable inputs, and for plants with improved responses to various stresses.

### BRIEF DESCRIPTION OF THE DRAWINGS

FIG. 1 shows exemplary results of endophyte MIC-28421 treatment on average corn shoot wet weight (grams) and grain weight (grams) relative to untreated corn grown in the same water deficit conditions.

FIG. 2 shows exemplary results of endophyte MIC-28421 treatment on leaf secondary nutrient content of corn plants grown in water deficit conditions. Nutrient content was measured 85 days after planting. Results show the average nutrient content per treatment as a percentage of total weight. The first row of graphs show average leaf calcium content per treatment. The second row of graphs show average leaf magnesium content per treatment, where * indicates a statistically significant difference between plants treated with MIC-28421 and untreated controls. The third row of graphs show average leaf magnesium content per treatment, where * indicates a statistically significant difference between plants treated with MIC-28421 and untreated controls.

FIG. 3 shows exemplary images of representative ears indicative of the treatment mean for MIC-28421 and untreated control plants grown in stress conditions. The representative ear of a corn plant treated with MIC-28421 is larger and has more fully developed kernels than that of the untreated control corn plant.

FIG. 4A shows exemplary results of endophyte MIC-28421 treatment on field grown corn plants under water stress during grain fill. The values shown are the difference in yield (bushels/acre) between corn treated with MIC-28421 and untreated controls. Error bars indicate 95% confidence intervals.

FIG. 4B shows exemplary results of endophyte MIC-28421 treatment on field grown corn plants not under water stress. The values shown are the difference in yield (bushels/acre) between corn treated with MIC-28421 and untreated controls. Error bars indicate 95% confidence intervals.

FIG. 5A shows exemplary results of endophyte treatment with the combination of MIC-28421 and MIC-93265 on corn in field trials under water stress during grain fill and flowering. The values shown are the difference in yield (bushels/acre) between corn treated with the combination of MIC-28421 and MIC-93265 and untreated controls. Error bars indicate 95% confidence intervals.

FIG. 5B shows exemplary results of endophyte treatment with the combination of MIC-28421 and MIC-93265 on corn in field trials not under water stress. The values shown are the difference in yield (bushels/acre) between corn treated with the combination of MIC-28421 and MIC-93265 and untreated controls. Error bars indicate 95% confidence intervals.

FIG. 6 shows exemplary results of endophyte treatment of the combination of MIC-28421 and MIC-93265, as well as MIC-28421 and MIC-93265 individually on corn in field trials. The values shown are total yield as kilograms/hectare (kg/ha).

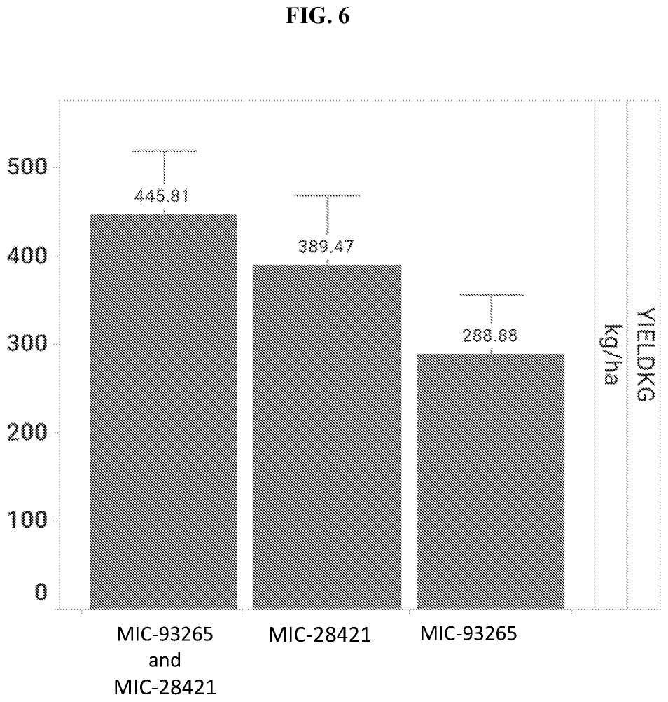

FIG. 7A shows exemplary results of endophyte treatment of the combination of MIC-28421 and MIC-93265, as well as MIC-28421 and MIC-93265 individually on corn in field trials under intermediate drought stress at flowering stage. The symbol * represents treatments having a greater than 99% probability that the yield uplift is positive. Yield uplift (kg/ha) represents the difference between each treatment and the untreated controls.

FIG. 7B shows exemplary results of endophyte treatment of the combination of MIC-28421 and MIC-93265, as well as MIC-28421 and MIC-93265 individually on corn in field trials under high drought stress at grain filling stage. The symbol * represents treatments having a greater than 99% probability that the yield uplift is positive. Yield uplift (kg/ha) represents the difference between each treatment and the untreated controls.

FIG. 8A shows exemplary results of endophyte treatment of the combination of MIC-28421 and MIC-93265 on corn in field trials a variety of stress conditions and soil types. Yield uplift (kg/ha) represents the difference between the endophyte treatment and the untreated controls.

FIG. 8B shows exemplary results of endophyte treatment of MIC-93265 on corn in field trials a variety of stress conditions and soil types. Yield uplift (kg/ha) represents the difference between the endophyte treatment and the untreated controls.

FIG. 8C shows exemplary results of endophyte treatment of MIC-28421 on corn in field trials in a variety of stress conditions and soil types. Yield uplift (kg/ha) represents the difference between the endophyte treatment and the untreated controls.

### SUMMARY OF INVENTION

In some embodiments, the invention provides a method of improving plant health, comprising heterologously disposing one or more endophytes to a plant element in an effective amount to improve a trait of agronomic importance in a plant derived from the treated plant element relative to a reference plant derived from a reference plant element, wherein the one or more endophytes comprise at least one polynucleotide sequence that is at least 97% identical to SEQ ID NO. 29-37, or 38. In some embodiments, the plant element is a monocot. In some embodiments, the monocot is a cereal. In some embodiments, the cereal is selected from the group consisting of wheat, rice, barley, buckwheat, rye, millet, oats, corn, sorghum, triticale and spelt. In some embodiments, the cereal is wheat. In some embodiments, the plant element is a dicot. In some embodiments, the dicot is selected from the group consisting of cotton, canola, sunflower, tomato, lettuce, peppers, cucumber, endive, melon, potato, and squash. In some embodiments, the dicot is a legume. In some embodiments, the legume is soy, peanut, peas, or beans. In some embodiments, the plant element is a whole plant, seedling, meristematic tissue, ground tissue, vascular tissue, dermal tissue, seed, leaf, root, shoot, stem, flower, fruit, stolon, bulb, tuber, corm, keikis, or bud. In some embodiments, the plant element is a seed. In some embodiments, the method additionally comprises the step of placing the plant elements in or on a growth medium. In some embodiments, the one or more endophytes are heterologously disposed to a plant element prior to placing the treated plant element in or on a growth medium. In some embodiments, the one or more endophytes are heterologously disposed to a plant element after placing the plant elements in or on a growth medium. In some embodiments, the one or more endophytes are heterologously disposed to a plant element concurrently with placing the plant elements in or on a growth medium. In some embodiments, the one or more endophytes are heterologously disposed to a plant element at least two times. In some embodiments, the one or more endophytes are heterologously disposed to a plant element via a seed treatment or soil pre-treatment and one or more foliar applications. In some embodiments, the one or more endophytes are heterologously disposed to a plant element via a seed treatment or soil pre-treatment and one or more floral applications. In some embodiments, the one or more endophytes are heterologously disposed to a plant element via one or more seed treatments or soil pre-treatments, one or more foliar applications, and one or more floral applications.

In some embodiments, the one or more endophytes are heterologously disposed to a plant element via seed treatment, on-planter application, root wash, seedling soak, foliar application, floral application, soil inoculum, in-furrow application, sidedress application, soil pre-treatment, wound inoculation, drip tape irrigation, vector-mediation inoculation, injection, osmopriming, hydroponics, aquaponics, or aeroponics. In some embodiments, the one or more endophytes are heterologously disposed to a plant element of a different plant variety from the variety of the plant element from which the one or more endophytes were obtained. In some embodiments, the one or more endophytes are heterologously disposed to a plant element of the same plant variety as the variety of the plant element from which the one or more endophytes were obtained. In some embodiments, the one or more endophytes are heterologously disposed to a plant element of a different plant species from the species of the plant element from which the one or more endophytes were obtained. In some embodiments, the one or more endophytes are heterologously disposed to a plant element of the same plant species as the species of the plant element from which the one or more endophytes were obtained. In some embodiments, the plant elements are allowed to germinate. In some embodiments, the plant elements are grown to yield. In some embodiments, the trait of agronomic importance is selected from the group consisting of drought tolerance, heat tolerance, cold tolerance, salinity tolerance, metal tolerance, herbicide tolerance, improved water use efficiency, improved nitrogen utilization, improved nitrogen fixation, improved nutrient use efficiency, improved nutrient utilization, improved phosphorus use efficiency, biotic stress tolerance, yield improvement, health enhancement, vigor improvement, decreased necrosis, decreased chlorosis, decreased area of necrotic tissue, decreased area of chlorotic tissue, growth improvement, photosynthetic capability improvement, nutrition enhancement, altered protein content, altered oil content, increased biomass, increased shoot height, increased root length, increased shoot biomass, increased root biomass, increased leaf area, increased shoot area, increased root area, increased seedling area, improved root architecture, increased seed germination percentage, increased seed germination rate, increased seedling survival, increased survival, photosynthetic efficiency, transpiration rate, seed/fruit number or mass, plant grain or fruit yield, leaf chlorophyll content, photosynthetic rate, wilt recovery, turgor pressure, modulation of a metabolite, production of a volatile organic compound (VOC), modulation of the proteome, increased seed weight, altered seed carbohydrate composition, altered seed oil composition, altered seed protein composition, altered seed nutrient composition, and combinations thereof. In some embodiments, the trait of agronomic importance is improved nutrient use efficiency. In some embodiments, the trait of agronomic importance is drought tolerance. In some embodiments, the plant element is wheat, and the trait of agronomic importance is one of more of increased root length, increased yield, and increased shoot length. In some embodiments, the plant element is corn, and the trait of agronomic importance is one of more of total grain weight, nutrition enhancement, shoot fresh weight, increased yield, increased win rate, and improved grain fill. In some embodiments, the plant element is rice, and the trait of agronomic importance is one or more of increased shoot dry weight, and tiller number. In some embodiments, the trait of agronomic importance is nutrition enhancement. In some embodiments, the nutrition enhancement comprises an increase in magnesium (Mg) or sulfur (S).

In some embodiments, the nutrition enhancement comprises an increase in magnesium (Mg) or sulfur (S) in leaf tissue.

In some embodiments, the invention provides a synthetic composition, comprising one or more endophytes heterologously disposed to a treatment formulation, wherein the one or more endophytes comprise at least one polynucleotide sequence that is at least 97% identical to SEQ ID NO. 29-37, or 38. In some embodiments, the composition additionally comprises a plant element. In some embodiments, the one or more endophytes are capable of improving a trait of agronomic importance in a plant derived from the plant element relative to a plant derived from a reference plant element. In some embodiments, the plant element is a monocot. In some embodiments, the monocot is a cereal. In some embodiments, the cereal is wheat. In some embodiments, the plant element is a dicot. In some embodiments, the dicot is selected from the group consisting of cotton, canola, sunflower, tomato, lettuce, peppers, cucumber, endive, melon, potato, and squash. In some embodiments, the dicot is a legume. In some embodiments, the legume is soy, peanut, peas, or beans. In some embodiments, the trait of agronomic importance is selected from the group consisting of drought tolerance, heat tolerance, cold tolerance, salinity tolerance, metal tolerance, herbicide tolerance, improved water use efficiency, improved nitrogen utilization, improved nitrogen fixation, improved nutrient use efficiency, improved nutrient utilization, improved phosphorus use efficiency, biotic stress tolerance, yield improvement, health enhancement, vigor improvement, decreased necrosis, decreased chlorosis, decreased area of necrotic tissue, decreased area of chlorotic tissue, growth improvement, photosynthetic capability improvement, nutrition enhancement, altered protein content, altered oil content, increased biomass, increased shoot height, increased root length, increased shoot biomass, increased root biomass, increased leaf area, increased shoot area, increased root area, increased seedling area, improved root architecture, increased seed germination percentage, increased seed germination rate, increased seedling survival, increased survival, photosynthetic efficiency, transpiration rate, seed/fruit number or mass, plant grain or fruit yield, leaf chlorophyll content, photosynthetic rate, wilt recovery, turgor pressure, modulation of a metabolite, production of a volatile organic compound (VOC), modulation of the proteome, increased seed weight, altered seed carbohydrate composition, altered seed oil composition, altered seed protein composition, altered seed nutrient composition, and combinations thereof. In some embodiments, the trait of agronomic importance is biotic stress tolerance. In some embodiments, the trait of agronomic importance is improved nutrient use efficiency. In some embodiments, the trait of agronomic importance is drought tolerance. In some embodiments, the synthetic composition additionally comprises one or more of a surfactant, a buffer, a tackifier, a microbial stabilizer, a fungicide, an anticomplex agent, an herbicide, a nematicide, an insecticide, a plant growth regulator, a rodenticide, a desiccant, a nutrient, an excipient, a wetting agent, a salt, and a polymer. In some embodiments, the polymer is a biodegradable polymer selected from the group consisting of alginate, agarose, agar, gelatin, polyacrylamide, chitosan, polyvinyl alcohol, and combinations thereof.

In some embodiments, the biodegradable polymer is alginate and the alginate is sodium alginate. In some embodiments, the one or more endophytes are encapsulated in polymeric beads. In some embodiments, the polymeric beads are less than 500 μm in diameter at their widest point. In some embodiments, the polymeric beads are less than 200 μm in diameter at their widest point. In some embodiments, the polymeric beads are less than 100 μm in diameter at their widest point. In some embodiments, the polymeric beads are less than 50 μm in diameter at their widest point. In some embodiments, the polymeric beads' average diameter at their widest point is between 500 μm and 250 μm. In some embodiments, the polymeric beads' average diameter at their widest point is between 249 μm and 100 μm. In some embodiments, the polymeric beads' average diameter at their widest point is between 100 μm and 50 μm. In some embodiments, the synthetic composition may be stored at between 0° C. and 4° C. for 1 week with less than 1 log loss of CFU of the one or more endophytes. In some embodiments, the synthetic composition may be stored at between 4.1° C. and 20° C. for 1 week with less than 1 log loss of CFU of the one or more endophytes. In some embodiments, the synthetic composition may be stored at between 20.1° C. and 33° C. for 1 week with less than 1 log loss of CFU of the one or more endophytes.

In some embodiments, the invention provides a method of measuring plant health, comprising determining the presence or abundance of one or more endophytes in a plant element, growth medium or growth environment, wherein the one or more endophytes comprise at least one polynucleotide sequence that is at least 97% identical to SEQ ID NO. 29-37, or 38. In some embodiments, the presence or abundance of one or more endophytes is determined relative to a reference plant element, growth medium or growth environment. In some embodiments, the one or more endophytes are not present in the reference plant element, growth medium or growth environment. In some embodiments, the one or more endophytes are less abundant in the reference plant element, growth medium or growth environment. In some embodiments, the presence or abundance of one or more endophytes is determined in a plant element and modulation of one or more traits of agronomic importance is inferred from the presence or amount of the one or more endophytes in the plant element. In some embodiments, the presence or abundance of one or more endophytes is determined in a growth medium and the capacity of the growth medium to modulate one or more trait of agronomic importance in a plant element planted therein is inferred from the presence or amount of the one or more endophytes in the growth medium.

In some embodiments, the presence or abundance of one or more endophytes is determined in a growth environment and the capacity of the growth environment to modulate one or more trait of agronomic importance in a plant element grown therein is inferred from the presence or amount of the one or more endophytes in the growth environment. In some embodiment, the trait of agronomic importance is selected from the group consisting of drought tolerance, heat tolerance, cold tolerance, salinity tolerance, metal tolerance, herbicide tolerance, improved water use efficiency, improved nitrogen utilization, improved nitrogen fixation, improved nutrient use efficiency, improved nutrient utilization, improved phosphorus use efficiency, biotic stress tolerance, yield improvement, health enhancement, vigor improvement, decreased necrosis, decreased chlorosis, decreased area of necrotic tissue, decreased area of chlorotic tissue, growth improvement, photosynthetic capability improvement, nutrition enhancement, altered protein content, altered oil content, increased biomass, increased shoot height, increased root length, increased shoot biomass, increased root biomass, increased leaf area, increased shoot area, increased root area, increased seedling area, improved root architecture, increased seed germination percentage, increased seed germination rate, increased seedling survival, increased survival, photosynthetic efficiency, transpiration rate, seed/fruit number or mass, plant grain or fruit yield, leaf chlorophyll content, photosynthetic rate, wilt recovery, turgor pressure, modulation of a metabolite, production of a volatile organic compound (VOC), modulation of the proteome, increased seed weight, altered seed carbohydrate composition, altered seed oil composition, altered seed protein composition, altered seed nutrient composition, and combinations thereof.

In some embodiments, the plant element is a monocot. In some embodiments, the monocot is a cereal. In some embodiments, the cereal is selected from the group consisting of wheat, rice, barley, buckwheat, rye, millet, oats, corn, sorghum, triticale and spelt. In some embodiments, the cereal is wheat. In some embodiments, the plant element is a dicot. In some embodiments, the dicot is selected from the group consisting of cotton, canola, sunflower, tomato, lettuce, peppers, cucumber, endive, melon, potato, and squash. In some embodiments, the dicot is a legume. In some embodiments, the legume is soy, peanut, peas, or beans. In some embodiments, a plurality of nucleic acid probes is used to determine the presence or abundance of one or more endophytes in a plant element, growth medium or growth environment, wherein the plurality comprises complementary or reverse complementary sequences to a region of at least 10 contiguous nucleotides within SEQ ID NO. 29-37, or 38. In some embodiments, the complementary or reverse complementary region comprises at least 20 contiguous nucleotides. In some embodiments, the complementary or reverse complementary region comprises at least 30 contiguous nucleotides. In some embodiments, the complementary or reverse complementary region comprises at least 40 contiguous nucleotides. In some embodiments, the plurality of nucleic acid probes is single-stranded DNA. In some embodiments, the plurality of nucleic acid probes is attached to one or more solid supports. In some embodiments, the plurality of nucleic acid probes is attached to a plurality of beads. In some embodiments, the plurality of nucleic acid probes is attached to a contiguous solid support. In some embodiments, the presence or abundance of one or more endophytes is determined by polymerase chain reaction, fluorescence in situ hybridization, or isothermal amplification.

### DETAILED DESCRIPTION

Terms used in the claims and specification are defined as set forth below unless otherwise specified.

It must be noted that, as used in the specification and the appended claims, the singular forms “a,” “an” and “the” include plural referents unless the context clearly dictates otherwise.

This invention relates to methods and compositions for improving plant health. The present invention includes methods for improving plant health, as well as synthetic compositions comprising endophytes capable of improving plant health, and nucleic acid probes and nucleic acid detection kits that may be used to identify endophytes of the present invention.

“Plant health” is demonstrated by the improvement of a trait of agronomic importance in a plant or plant element as compared to a reference plant or plant element. A trait of agronomic importance includes, but is not limited to, drought tolerance, heat tolerance, cold tolerance, salinity tolerance, metal tolerance, herbicide tolerance, improved water use efficiency, improved nitrogen utilization, improved nitrogen fixation, improved nutrient use efficiency, improved nutrient utilization, improved phosphorus use efficiency, biotic stress tolerance, yield improvement, health enhancement, vigor improvement, decreased necrosis, decreased chlorosis, decreased area of necrotic tissue, decreased area of chlorotic tissue, decreased pathogen load of tissues, growth improvement, photosynthetic capability improvement, nutrition enhancement, altered protein content, altered oil content, increased biomass, increased shoot height, increased root length, increased shoot biomass, increased root biomass, increased leaf area, increased shoot area, increased root area, increased seedling area, improved root architecture, increased seed germination percentage, increased seed germination rate, increased seedling survival, increased survival, photosynthetic efficiency, transpiration rate, seed/fruit number or mass, plant grain or fruit yield, leaf chlorophyll content, photosynthetic rate, wilt recovery, turgor pressure, production of a volatile organic compound (VOC), increased seed weight, altered seed carbohydrate composition, altered seed oil composition, altered seed protein composition, altered seed nutrient composition, and combinations thereof. The phrase “biotic stress” refers to a growth environment comprising one or more pests or pathogens. Pests can be nematodes and/or insects. In some embodiments, a pest is of the order Lepidoptera, Hemiptera, Tylenchida/Rhabditida, Dorylaimida, Trichinellida, or Triplonchida. In some embodiments, a pest is of a genera Chrysodeixis, Trichoplusia, Nezara, Lygus, Aphis, Belonolaimus, Xiphenema, Trichodorus, Pratylenchus, Aphelenchoides, Meloidogyne, or Rotylenchulus. Pathogens can be fungal, viral, protist, or bacterial pathogens, for example, pathogens of vertebrates or plants. In some embodiments, a pathogen is of a genera Pythium, Rhizoctonia, Phytophthora, Fusarium, Alternaria, Stagonospora, Aspergillus, Magnaporthe, Botrytis, Puccinia, Blumeria, Erysiphe, Leveillula, Mycosphaerella, or Colletotrichum.

“Biomass” means the total mass or weight (fresh or dry), at a given time (for example, age or stage of development), of a plant tissue, plant tissues, an entire plant, or population of plants. The term may also refer to all the plants or species in the community (“community biomass”).

An “increased yield” can refer to any increase in seed or fruit biomass; or seed, seed pod or ear, or fruit number per plant; or seed or fruit weight; or seed or fruit size per plant or unit of production area, e.g., acre or hectare. For example, increased yield of seed or fruit biomass may be measured in units of bushels per acre, pounds per acre, tons per acre, or kilos per hectare. An increased yield can also refer to an increased production of a component of, or product derived from, a plant or plant element or of a unit of measure thereof (for example, increased carbohydrate yield of a grain or increased oil yield of a seed). Typically, where yield indicates an increase in a particular component or product derived from a plant, the particular characteristic is designated when referring to increased yield, e.g., increased oil or grain yield or increased protein yield or seed size.

“Nutrition enhancement” refers to modulation of the presence, abundance or form of one or more substances in a plant element, wherein the modulation of the one or more substances provides a benefit to other organisms that consume or utilize said plant element.

Synthetic compositions and methods of use described herein may improve plant health by providing an improved benefit or tolerance to a plant that is of at least 0.1%, at least 0.5%, at least 1%, at least 2%, at least 3%, between 3% and 5%, at least 5%, between 5% and 10%, at least 10%, between 10% and 15%, at least 15%, between 15% and 20%, at least 20%, between 20% and 30%, at least 30%, between 30% and 40%, at least 40%, between 40% and 50%, at least 50%, between 50% and 60%, at least 60%, between 60% and 75%, at least 75%, between 75% and 100%, at least 100%, between 100% and 150%, at least 150%, between 150% and 200%, at least 200%, between 200% and 300%, at least 300% or more, when compared with a reference plant. A “reference plant”, “reference plant element”, “reference agricultural plant”, or “reference seed” means a similarly situated plant or seed of the same species, strain, or cultivar to which a treatment, formulation, composition or endophyte preparation as described herein is not administered/contacted. A reference plant, therefore, is identical to the treated plant except for the presence of the active ingredient to be tested and can serve as a control for detecting the effects of the treatment conferred to the plant. A plurality of reference plants may be referred to as a “reference population”.

In some embodiments, one or more endophytes and or one or more compounds produced by one or more endophytes are heterologously disposed on a plant element in an effective amount to improve plant health. In some embodiments, an improvement of plant health is measured by an increase in a trait of agronomic importance, for example root length or yield. In some embodiments, an improvement of subject health is measured by a decrease in a trait of importance, for example necrosis or chlorosis. In some embodiments, improved plant health is demonstrated by an improvement of a trait of agronomic importance or tolerance in a treated plant by at least 0.1%, at least 0.5%, at least 1%, at least 2%, at least 3%, between 3% and 5%, at least 5%, between 5% and 10%, at least 10%, between 10% and 15%, at least 15%, between 15% and 20%, at least 20%, between 20% and 30%, at least 30%, between 30% and 40%, at least 40%, between 40% and 50%, at least 50%, between 50% and 60%, at least 60%, between 60% and 75%, at least 75%, between 75% and 100%, at least 100%, between 100% and 150%, at least 150%, between 150% and 200%, at least 200%, between 200% and 300%, at least 300% or more, as compared to a reference plant element not further comprising said endophyte. An “effective amount” of one or more endophytes is the amount capable of improving trait of agronomic importance or tolerance by at least 0.1%, at least 0.5%, at least 1%, at least 2%, at least 3%, between 3% and 5%, at least 5%, between 5% and 10%, at least 10%, between 10% and 15%, at least 15%, between 15% and 20%, at least 20%, between 20% and 30%, at least 30%, between 30% and 40%, at least 40%, between 40% and 50%, at least 50%, between 50% and 60%, at least 60%, between 60% and 75%, at least 75%, between 75% and 100%, at least 100%, between 100% and 150%, at least 150%, between 150% and 200%, at least 200%, between 200% and 300%, at least 300% or more, as compared to a reference plant element not further comprising said endophyte. In some embodiments, an effective amount of treatment comprising an endophyte is at least 10 CFU per unit of plant element, at least 10\hat{ }2 CFU per unit of plant element, between 10\hat{ }2 and 10\hat{ }3 CFU per unit of plant element, at least about 10\hat{ }3 CFU per unit of plant element, between 10\hat{ }3 and 10\hat{ }4 CFU per unit of plant element, at least about 10\hat{ }4 CFU per unit of plant element, between 10\hat{ }4 and 10\hat{ }5 CFU per unit of plant element, at least about 10\hat{ }5 CFU, between 10\hat{ }5 and 10\hat{ }6 CFU per unit of plant element, at least about 10\hat{ }6 CFU per unit of plant element, between 10\hat{ }6 and 10\hat{ }7 CFU per unit of plant element, at least about 10\hat{ }7 CFU per unit of plant element, between 10\hat{ }7 and 10\hat{ }8 CFU per unit of plant element, or even greater than 10\hat{ }8 CFU per unit of plant element. A unit of a plant element may be an individual plant element, e.g., an individual seed, or a unit of surface area of a plant element, e.g., a square inch of leaf tissue, or unit of surface area of a plant element, e.g., a cubic centimeter of root.

The methods and compositions of the present invention are broadly applicable to cultivated plants, particularly plants that are cultivated by humans for food, feed, fiber, fuel, and/or industrial purposes. In some embodiments, plants (including seeds and other plant elements) are monocots or dicots. In some embodiments, plants used in the methods and compositions of the present invention include, but are not limited to: agricultural row, agricultural grass plants or other field crops: wheat, rice, barley, buckwheat, beans (for example: soybean, snap, dry), corn (for example: grain, seed, sweet corn, silage, popcorn, high oil), canola, peas (for example: dry, succulent), peanuts, safflower, sunflower, alfalfa hay, forage and cover crops (for example: alfalfa, clover, vetch, and trefoil), berries and small fruits (for example: blackberries, blueberries, currants, elderberries, gooseberries, huckleberries, loganberries, raspberries, strawberries, bananas and grapes), bulb crops (for example: garlic, leeks, onions, shallots, and ornamental bulbs), citrus fruits (for example: citrus hybrids, grapefruit, kumquat, limes, oranges, and pummelos), cucurbit vegetables (for example: cucumbers, melons, gourds, pumpkins, and squash), flowers (for example: ornamental, horticultural flowers including roses, daisies, tulips, freesias, carnations, heather, lilies, irises, orchids, snapdragons, and ornamental sunflowers), bedding plants, ornamentals, fruiting vegetables (for example: eggplant, sweet and hot peppers, tomatillos, and tomatoes), herbs, spices, mints, hydroponic crops (for example: cucumbers, tomatoes, lettuce, herbs, and spices), leafy vegetables and cole crops (for example: arugula, celery, chervil, endive, fennel, lettuce including head and leaf, parsley, radicchio, rhubarb, spinach, Swiss chard, broccoli, Brussels sprouts, cabbage, cauliflower, collards, kale, kohlrabi, and mustard greens), asparagus, legume vegetable and field crops (for example: snap and dry beans, lentils, succulent and dry peas, and peanuts), pome fruit (for example: pears and quince), root crops (for example: beets, sugar beets, red beets, carrots, celeriac, chicory, horseradish, parsnip, radish, rutabaga, salsify, and turnips), deciduous trees (for example: maple and oak), evergreen trees (for example: pine, cedar, hemlock and spruce), small grains (for example: rye, wheat including spring and winter wheat, millet, oats, barley including spring and winter barley, and spelt), stone fruits (for example: apricots, cherries, nectarines, peaches, plums, and prunes), tree nuts (for example: almonds, beech nuts, Brazil nuts, butternuts, cashews, chestnuts, filberts, hickory nuts, macadamia nuts, pecans, pistachios, and walnuts), and tuber crops (for example: potatoes, sweet potatoes, yams, artichoke, cassava, and ginger). In a particular embodiment, the agricultural plant is selected from the group consisting of rice (Oryza sativa and related varieties), soy (Glycine max and related varieties), wheat (Triticum aestivum and related varieties), oats (Avena sativa and related varieties), barley (Hordeum vulgare and related varieties), corn (Zea mays and related varieties), peanuts (Arachis hypogaea and related varieties), canola (Brassica napus, Brassica rapa and related varieties), sunflower (Helianthus spp.), coffee (Coffea spp.), cocoa (Theobroma cacao), melons, and tomatoes (Solanum lycopsersicum and related varieties).

Plant health may be improved by treatment of a plant or plant element. A “plant element” is intended to generically reference either a whole plant or a plant component, including but not limited to plant tissues, parts, and cell types. A plant element is preferably one of the following: whole plant, seedling, meristematic tissue, ground tissue, vascular tissue, dermal tissue, seed, leaf, root, shoot, stem, flower, fruit, stolon, bulb, tuber, corm, keikis, or bud.

Plant health may be improved by treatment with a composition of the present invention, in particular compositions of the present invention comprising one or more endophytes. An “endophyte” is an organism capable of living on a plant element (e.g., rhizoplane or phyllosphere) or within a plant element, or on a surface in close physical proximity with a plant element, e.g., the phyllosphere and rhizosphere including soil surrounding roots. A “beneficial” endophyte does not cause disease or harm the host plant otherwise. Endophytes can occupy the intracellular or extracellular spaces of plant tissue, including the leaves, stems, flowers, fruits, seeds, or roots. An endophyte can be, for example, a bacterial or fungal organism, and can confer a beneficial property to the host plant such as an increase in yield, biomass, resistance, or fitness. An endophyte can be a fungus or a bacterium. As used herein, the term “microbe” is sometimes used to describe an endophyte. As used herein, the term “microbe” or “microorganism” refers to any species or taxon of microorganism, including, but not limited to, archaea, bacteria, microalgae, fungi (including mold and yeast species), mycoplasmas, microspores, nanobacteria, oomycetes, and protozoa. In some embodiments, a microbe or microorganism is an endophyte, for example a bacterial or fungal endophyte, which is capable of living within a plant.

The term “isolated” is intended to specifically reference an organism, cell, tissue, polynucleotide, or polypeptide that is removed from its original source and purified from additional components with which it was originally associated. For example, an endophyte may be considered isolated from a seed if it is removed from that seed source and purified so that it is isolated from one or more additional components with which it was originally associated. Similarly, an endophyte may be removed and purified from a plant or plant element so that it is isolated and no longer associated with its source plant or plant element.

As used herein, an isolated strain of a microbe is a strain that has been removed from its natural milieu. “Pure cultures” or “isolated cultures” are cultures in which the organisms present are only of one strain of a particular genus and species. This is in contrast to “mixed cultures,” which are cultures in which more than one genus and/or species of microorganism are present. As such, the term “isolated” does not necessarily reflect the extent to which the microbe has been purified. A “substantially pure culture” of the strain of microbe refers to a culture which contains substantially no other microbes than the desired strain or strains of microbe. In other words, a substantially pure culture of a strain of microbe is substantially free of other contaminants, which can include microbial contaminants. Further, as used herein, a “biologically pure” strain is intended to mean the strain was separated from materials with which it is normally associated in nature. A strain associated with other strains, or with compounds or materials that it is not normally found with in nature, is still defined as “biologically pure.” A monoculture of a particular strain is, of course, “biologically pure.” As used herein, the term “enriched culture” of an isolated microbial strain refers to a microbial culture that contains more than 50%, 60%, 70%, 80%, 90%, or 95% of the isolated strain.

A “population” of endophytes, or an “endophyte population”, refers to one or more endophytes that share a common genetic derivation, e.g., one or more propagules of a single endophyte, i.e., endophytes grown from a single picked colony. In some embodiments, a population refers to endophytes of identical taxonomy. In some cases, a population of endophytes refers to one or more endophytes of the same genus. In some cases, a population of endophytes refers to one or more endophytes of the same species or strain.

A “plurality of endophytes” means two or more types of endophyte entities, e.g., bacteria or fungi, or combinations thereof. In some embodiments, the two or more types of endophyte entities are two or more individual endophytic organisms, regardless of genetic derivation or taxonomic relationship. In some embodiments, the two or more types of endophyte entities are two or more populations of endophytes. In other embodiments, the two or more types of endophyte entities are two or more species of endophytes. In yet other embodiments, the two or more types of endophyte entities are two or more genera of endophytes. In yet other embodiments, the two or more types of endophyte entities are two or more families of endophytes. In yet other embodiments, the two or more types of endophyte entities are two or more orders of endophytes. In yet other embodiments, the two or more types of endophyte entities are two or more classes of endophytes. In yet other embodiments, the two or more types of endophyte entities are two or more phyla of endophytes. In some embodiments, a plurality refers to three or more endophytes, either distinct individual organisms or distinct members of different genetic derivation or taxa. In some embodiments, a plurality refers to four or more, either distinct individual endophytic organisms or distinct members of different genetic derivation or taxa. In some embodiments, a plurality refers to five or more, ten or more, or an even greater number of either distinct individual endophytic organisms or distinct members of different genetic derivation or taxa. In some embodiments, the term “consortium” or “consortia” may be used as a collective noun synonymous with “plurality”, when describing more than one population, species, genus, family, order, class, or phylum of endophytes.

In some embodiments, a treatment may comprise a modified microbe, plant or plant element. A microbe, plant or plant element is “modified” when it comprises an artificially introduced genetic or epigenetic modification. In some embodiments, the modification is introduced by genome engineering or genome editing technology. In some embodiments, genome engineering or editing utilizes non-homologous end joining (NHEJ), homology directed repair (HDR), or combinations thereof. In some embodiments, genome engineering or genome editing is carried out with a Class I or Class II clustered regulatory interspaced short palindromic repeats (CRISPR) system. In some embodiments, the CRISPR system is CRISPR/Cas9. In some embodiments, the CRISPR system is CRISPR/Cpf1. In some embodiments, the modification is introduced by a targeted nuclease. In some embodiments, targeted nucleases include, but are not limited to, transcription activator-like effector nuclease (TALEN), zinc finger nuclease (ZNF), Cas9, Cas9 variants, Cas9 homologs, Cpf1, Cpf1 variants, Cpf1 homologs, and combinations thereof. In some embodiments, the modification is an epigenetic modification. In some embodiments, the modification is introduced by treatment with a DNA methyltransferase inhibitor such as 5-azacytidine, or a histone deacetylase inhibitor such as 2-amino-7-methoxy-3H-phenoxazin-3-one. In some embodiments, the modification is introduced via tissue culture. In some embodiments, a modified microbe, plant or plant element comprises a transgene.

As used herein, the term “bacterium” or “bacteria” refers in general to any prokaryotic organism and may reference an organism from either Kingdom Eubacteria (Bacteria), Kingdom Archaebacteria (Archaea), or both. In some cases, bacterial genera have been reassigned due to various reasons (such as, but not limited to, the evolving field of whole genome sequencing), and it is understood that such nomenclature reassignments are within the scope of any claimed genus.

As used herein, the term “fungus” or “fungi” refers in general to any organism from Kingdom Fungi. Historical taxonomic classification of fungi has been according to morphological presentation. Beginning in the mid-1800's, it was recognized that some fungi have a pleomorphic life cycle, and that different nomenclature designations were being used for different forms of the same fungus. With the development of genomic sequencing, it became evident that taxonomic classification based on molecular phylogenetics did not align with morphological-based nomenclature (Shenoy B D, Jeewon R, Hyde K D. Impact of DNA sequence-data on the taxonomy of anamorphic fungi. Fungal Diversity 26(10) 1-54. 2007). Systematics experts have not aligned on common nomenclature for all fungi, nor are all existing databases and information resources inclusive of updated taxonomies. As such, many fungi provided herein may be described by their anamorph form, but it is understood that based on identical genomic sequencing, any pleomorphic state of that fungus may be considered the same organism. In some cases, fungal genera have been reassigned due to various reasons, and it is understood that such nomenclature reassignments are within the scope of any claimed genus.

The degree of relatedness between microbes may be inferred from the sequence similarity of one or more homologous polynucleotide sequences of the microbes. In some embodiments, the one or more homologous polynucleotide sequences are marker genes. As used herein, the term “marker gene” refers to a conserved genomic region comprising sequence variation among related organisms.

Examples of fungal marker genes that may be used for the present invention, include but are not limited to: internal transcribed spacer (“ITS”); 60S ribosomal protein L10 (“RPL10”); actin (“ACT”); beta-tubulin or tubulin (“BTUB2”, “TUB2”, or “TB”); DNA topoisomerase I (“TOP1”); glyceraldehyde-3-phosphate dehydrogenase (“GDP”); mini-chromosome maintenance protein 7 (“MCM7”); largest subunit of RNA polymerase II (“RPB1”); second largest subunit of RNA polymerase II (“RPB2”); ‘long’ subunit rRNA (“LSU”); phosphoglycerate kinase (“PGK”); actin (“ACT”); long subunit rRNA gene (“LSU”); small subunit rRNA gene (“SSU”); translation elongation factor (“TEF1”); Calmodulin (“CMD”), etc.

The terms “sequence similarity”, “identity”, “percent identity”, “percent sequence identity” or “identical” in the context of polynucleotide sequences refer to the nucleotides in the two sequences that are the same when aligned for maximum correspondence. There are different algorithms known in the art that can be used to measure nucleotide sequence identity. Nucleotide sequence identity can be measured by a local or global alignment, preferably implementing an optimal local or optimal global alignment algorithm. For example, a global alignment may be generated using an implementation of the Needleman-Wunsch algorithm (Needleman, S. B. & Wunsch, C. D. (1970) Journal of Molecular Biology. 48(3):443-53). For example, a local alignment may be generated using an implementation of the Smith-Waterman algorithm (Smith T. F & Waterman, M. S. (1981) Journal of Molecular Biology. 147(1):195-197). Optimal global alignments using the Needleman-Wunsch algorithm and optimal local alignments using the Smith-Waterman algorithm are implemented in USEARCH, for example USEARCH version v8.1.1756_i86osx32.

A gap is a region of an alignment wherein a sequence does not align to a position in the other sequence of the alignment. A terminal gap is a region beginning at the end of a sequence in an alignment wherein the nucleotide in the terminal position of that sequence does not correspond to a nucleotide position in the other sequence of the alignment and extending for all contiguous positions in that sequence wherein the nucleotides of that sequence do not correspond to a nucleotide position in the other sequence of the alignment. An internal gap is a gap in an alignment which is flanked on the 3′ and 5′ end by positions wherein the aligned sequences are identical. In global alignments, terminal gaps are discarded before identity is calculated. For both local and global alignments, internal gaps are counted as differences.

In some embodiments, the nucleic acid sequence to be aligned is a complete gene. In some embodiments, the nucleic acid sequence to be aligned is a gene fragment. In some embodiments, the nucleic acid sequence to be aligned is an intergenic sequence. In a preferred embodiment, inference of homology from a sequence alignment is made where the region of alignment is at least 85% of the length of the query sequence.

The term “substantial homology” or “substantial similarity,” when referring to a polynucleotide sequence or fragment thereof, indicates that, when optimally aligned with appropriate nucleotide insertions or deletions with another polynucleotide sequence (or its complementary strand), there is nucleotide sequence identity in at least about 76%, 80%, 85%, or at least about 90%, or at least about 95%, 96%, at least 97%, 98%, 99% or 100% of the positions of the alignment, wherein the region of alignment is at least about 50%, 60%, 70%, 75%, 85%, or at least about 90%, or at least about 95%, 96%, 97%, 98%, 99% or 100% of the length of the query sequence. In a preferred embodiment, the region of alignment contains at least 100 positions inclusive of any internal gaps. In some embodiments, the region of alignment comprises at least 100 nucleotides of the query sequence. In some embodiments, the region of alignment comprises at least 200 nucleotides of the query sequence. In some embodiments, the region of alignment comprises at least 300 nucleotides of the query sequence. In some embodiments, the region of alignment comprises at least 400 nucleotides of the query sequence. In some embodiments, the region of alignment comprises at least 500 nucleotides of the query sequence. In some embodiments, the terminal nucleotides are trimmed from one or both ends of the sequence prior to alignment. In some embodiments, at least the terminal 10, 15, 20, 25, 30, between 20-30, 35, 40, 45, 50, between 25-50 nucleotides are trimmed from the sequence prior to alignment.

### Synthetic Compositions for Improving Plant Health

In some embodiments, a synthetic composition comprises one or more endophytes capable of improving plant health. A “synthetic composition” comprises one or more endophytes combined by human endeavor with a heterologously disposed plant element or a treatment formulation, said combination which is not found in nature. In some embodiments, a synthetic composition comprises one or more plant elements or formulation components combined by human endeavor with an isolated, purified endophyte composition. In some embodiments, synthetic composition refers to a plurality of endophytes in a treatment formulation comprising additional components with which said endophytes are not found in nature. An endophyte is “heterologously disposed” when mechanically or manually applied, artificially inoculated or disposed onto or into a plant element, seedling, plant or onto or into a plant growth medium or onto or into a treatment formulation so that the endophyte exists on or in the plant element, seedling, plant, plant growth medium, or formulation in a manner not found in nature prior to the application of the treatment, e.g., said combination which is not found in nature in that plant variety, at that time in development, in that tissue, in that abundance, or in that growth condition (for example, drought, flood, cold, nutrient deficiency, etc.).

A “treatment formulation” refers to one or more compositions that facilitate the stability, storage, and/or application of one or more endophytes. Treatment formulations may comprise any one or more agents such as: antioxidant, a pH modifier, surfactant, a bulking agent, a solid diluent, a tackifier, a microbial stabilizer, an antimicrobial, a fungicide, an anticomplex agent, an herbicide, a nematicide, an insecticide, a plant growth regulator, a rodenticide, a desiccant, a nutrient, an excipient, a wetting agent, a salt, a polymer. As used herein as a noun, a “treatment” may comprise one or more endophytes.

In some embodiments, a treatment formulation may comprise one or more polymeric beads comprising one or more endophytes. In some embodiments, a treatment formulation may consist of one or more polymeric beads comprising one or more endophytes. A polymeric bead may contain a biodegradable polymer such as alginate, agarose, agar, gelatin, polyacrylamide, chitosan, and polyvinyl alcohol. In some embodiments, the polymeric beads are less than 500 μm in diameter at their widest point. In some embodiments, the polymeric beads' average diameter at their widest point is between 500 μm and 250 μm, between 249 μm and 100 μm, 100 μm or less, between 100 μm and 50 μm, or 50 μm or less.

In some embodiments, an “agriculturally compatible carrier” can be used to formulate an agricultural formulation or other composition that includes a purified endophyte preparation. As used herein an “agriculturally compatible carrier” refers to any material, other than water, that can be added to a plant element without causing or having an adverse effect on the plant element (e.g., reducing seed germination) or the plant that grows from the plant element, or the like.

In some embodiments, the formulation can include a tackifier or adherent. Such agents are useful for combining the bacterial population of the invention with carriers that can contain other compounds (e.g., control agents that are not biologic), to yield a coating composition. Such compositions help create coatings around the plant or seed to maintain contact between the microbe and other agents with the plant or plant part. In some embodiments, adherents are selected from the group consisting of: alginate, gums, starches, lecithins, formononetin, polyvinyl alcohol, alkali formononetinate, hesperetin, polyvinyl acetate, cephalins, Gum Arabic, Xanthan Gum, Mineral Oil, Polyethylene Glycol (PEG), Polyvinyl pyrrolidone (PVP), Arabino-galactan, Methyl Cellulose, PEG 400, Chitosan, Polyacrylamide, Polyacrylate, Polyacrylonitrile, Glycerol, Triethylene glycol, Vinyl Acetate, Gellan Gum, Polystyrene, Polyvinyl, Carboxymethyl cellulose, Gum Ghatti, and polyoxyethylene-polyoxybutylene block copolymers.

The formulation can also contain a surfactant. Non-limiting examples of surfactants include nitrogen-surfactant blends such as Prefer 28 (Cenex), Surf-N(US), Inhance (Brandt), P-28 (Wilfarm) and Patrol (Helena); esterified seed oils include Sun-It II (AmCy), MSO (UAP), Scoil (Agsco), Hasten (Wilfarm) and Mes-100 (Drexel); and organo-silicone surfactants include Silwet L77 (UAP), Silikin (Terra), Dyne-Amie (Helena), Kinetic (Helena), Sylgard 309 (Wilbur-Ellis) and Century (Precision). In one embodiment, the surfactant is present at a concentration of between 0.01% v/v to 10% v/v. In another embodiment, the surfactant is present at a concentration of between 0.1% v/v to 1% v/v.

In certain cases, the formulation includes a microbial stabilizer. Such an agent can include a desiccant. As used herein, a “desiccant” can include any compound or mixture of compounds that can be classified as a desiccant regardless of whether the compound or compounds are used in such concentrations that they in fact have a desiccating effect on the liquid inoculant. Such desiccants are ideally compatible with the bacterial population used, and should promote the ability of the microbial population to survive application on the seeds and to survive desiccation. Examples of suitable desiccants include one or more of trehalose, sucrose, glycerol, and Methylene glycol. Other suitable desiccants include, but are not limited to, non-reducing sugars and sugar alcohols (e.g., mannitol or sorbitol). The amount of desiccant introduced into the formulation can range from about 5% to about 50% by weight/volume, for example, between about 10% to about 40%, between about 15% and about 35%, or between about 20% and about 30%.

In some embodiments the formulation includes, for example, solid carriers such as talc, fullers earth, bentonite, kaolin clay, pyrophyllite, bentonite, montmorillonite, diatomaceous earth, acid white soil, vermiculite, and pearlite, and inorganic salts such as ammonium sulfate, ammonium phosphate, ammonium nitrate, urea, ammonium chloride, and calcium carbonate. Also, organic fine powders such as wheat flour, wheat bran, and rice bran may be used. The liquid carriers include vegetable oils such as soybean oil and cottonseed oil, glycerol, ethylene glycol, polyethylene glycol, propylene glycol, polypropylene glycol, etc.

In some embodiments, the abundance of an endophyte can be estimated by methods well known in the art including, but not limited to, qPCR, community sequencing, flow cytometry, and/or counting colony-forming units. As used herein, a “colony-forming unit” (“CFU”) is used as a measure of viable microorganisms in a sample. A CFU is an individual viable cell capable of forming on a solid medium a visible colony whose individual cells are derived by cell division from one parental cell.

In some embodiments, the synthetic composition of the present invention comprises one or more of the following: antimicrobial, fungicide, nematicide, bactericide, insecticide, or herbicide.

In some embodiments, a treatment is applied mechanically or manually or artificially inoculated to a plant element in a seed treatment, on-planter application, root wash, seedling soak, foliar application, floral application, soil inoculum, in-furrow application, sidedress application, soil pre-treatment, wound inoculation, drip tape irrigation, vector-mediation via a pollinator, injection, osmopriming, hydroponics, aquaponics, aeroponics, and combinations thereof. Application to the plant may be achieved, for example, as a powder for surface deposition onto plant leaves, as a spray to the whole plant or selected plant element, as part of a drip to the soil or the roots, or as a coating onto the plant element prior to or after planting. Such examples are meant to be illustrative and not limiting to the scope of the invention.

In some embodiments, the invention described herein provides a synthetic composition comprising one or more endophytes capable of improving plant health, wherein the one or more endophytes are members of the Order Coniochaetales. In some embodiments, the one or more endophytes are members of the Family Coniochaetaceae. In some embodiments, the one or more endophytes are members of the Genus Coniochaeta. In some embodiments, the one or more endophytes are selected from Table 5. In some embodiments, the one or more endophytes comprise one or more a polynucleotide sequences at least 95%, at least 96%, at least 97%, at least 97%, at least 98%, at least 99%, or 100% identical to one or more polynucleotide sequences selected from the group consisting of SEQ ID NOs. 29-39.

In some embodiments, the invention described herein provides a synthetic composition comprising one or more endophytes capable of conferring improved plant health in an agricultural plant under stress, wherein the one or more endophytes are members of the Order Coniochaetales. In some embodiments, the one or more endophytes are members of the Family Coniochaetaceae. In some embodiments, the one or more endophytes are members of the Genus Coniochaeta. In some embodiments, the one or more endophytes comprise one or more a polynucleotide sequences 95%, 96%, 97%, at least 97%, at least 98%, at least 99%, or 100% identical to one or more polynucleotide sequences selected from the group consisting of SEQ ID NOs. 29-38.

In some embodiments, a synthetic composition, comprises one or more endophytes heterologously disposed to a plant element, wherein the one or more endophytes comprises a first endophyte of the Order Coniochaetales, and a second endophyte of the Order Bacillales. In some embodiments, a synthetic composition, comprises one or more endophytes heterologously disposed to a plant element, wherein the one or more endophytes comprises a first endophyte of the Family Coniochaetaceae, and a second endophyte of the Family Bacillaceae. In some embodiments, a synthetic composition, comprises one or more endophytes heterologously disposed to a plant element, wherein the one or more endophytes comprises a first endophyte of the Genus Coniochaeta, and a second endophyte of the Genus Bacillus. In some embodiments, a synthetic composition, comprises one or more endophytes heterologously disposed to a plant element, wherein the one or more endophytes comprises a first endophyte having at least one polynucleotide sequence that is at least 97% identical to SEQ ID NO. 29-37, or 38, and a second endophyte having at least one polynucleotide sequence that is at least 97% identical to SEQ ID NO. 39. In some embodiments, a synthetic composition, comprises one or more endophytes heterologously disposed to a plant element, wherein the one or more endophytes comprises a first endophyte having at least one polynucleotide sequence that is at least 99% identical to SEQ ID NO. 29-37, or 38, and a second endophyte having at least one polynucleotide sequence that is at least 99% identical to SEQ ID NO. 39. In some embodiments, a synthetic composition, comprises one or more endophytes heterologously disposed to a plant element, wherein the one or more endophytes comprises a first endophyte comprising the polynucleotide sequence SEQ ID NO. 29, and a second endophyte comprising the polynucleotide sequence SEQ ID NO. 39.

In some embodiments of any of the synthetic compositions described herein, the synthetic compositions comprise at least 2, 3, 4, 5, 6, 7, 8, 9, 10, 15 or 20 or more endophytes. In some embodiments, the one or more endophytes comprise at least 2, 3, 4, 5, 6, 7, 8, 9, 10, 15 or 20 or more endophytes. In some embodiments, the one or more endophytes are distinct individual organisms or distinct members of different genetic derivation or taxa.

### Methods for Improving Plant Health

In some embodiments, the invention provides methods of improving plant health comprising heterologously disposing one or more endophytes to a plant element in an effective amount to increase a trait of agronomic importance in the plant derived from the treated plant element relative to a plant derived from a reference plant element. In some embodiments, the one or more endophytes are a component of a treatment formulation. In some embodiments, the one or more endophytes are a component of a synthetic composition.

In some embodiments, the invention provides methods of improving plant health comprising creating any of the synthetic compositions described herein, wherein the synthetic composition comprises any of the plant elements of any of the plants described herein and any of the one or more endophytes described herein. In some embodiments, the synthetic composition additionally comprises a growth medium. In some embodiments, the growth medium is soil. In some embodiments, the method comprises a step of applying the synthetic composition to a growth medium. In some embodiments, the method comprises a step of germinating the plants. In some embodiments, the method comprises a step of growing the plants. In some embodiments, the method comprises a step of growing the plants to yield.

In some embodiments, the synthetic composition comprises any of the treatment formulations described herein and any of the one or more endophytes described herein. In some embodiments, the synthetic composition additionally comprises a growth medium or growth environment. A growth environment is a natural or artificially constructed surrounding, capable of supporting the life of a plant. In some embodiments, the growth medium is soil. In some embodiments, the growth medium is a culture fluid suitable for propagation of an endophyte or plant tissue culture. In some embodiments, the method comprises a step of applying the synthetic composition to a growth medium. In some embodiments, the synthetic composition is applied before one or more plant elements are placed in or on the growth medium. In some embodiments, the synthetic composition is applied after one or more plant elements are placed in or on the growth medium. In some embodiments, the method comprises a step of germinating the plants. In some embodiments, the method comprises a step of growing the plants. In some embodiments, where the plants are commercially produced, maturity is the stage at which the plant is normally harvested.

In some embodiments of any of the methods described herein, plant health may be improved for plants in a stress condition. In some embodiments, the stress condition is a biotic or abiotic stress, or a combination of one or more biotic or abiotic stresses. In some embodiments of any of the methods described herein, the stress condition is an abiotic stress selected from the group consisting of drought stress, salt stress, metal stress, heat stress, cold stress, low nutrient stress (alternately referred to herein as nutrient deficiency or growth in nutrient deficient conditions), and excess water stress, and combinations thereof. In some embodiments of any of the methods described herein, the stress condition is a biotic stress selected from the group consisting of insect infestation, nematode infestation, complex infection, fungal infection, bacterial infection, oomycete infection, protozoal infection, viral infection, herbivore grazing, and combinations thereof. Stress tolerance is exemplified by improvement of one or more other traits of agronomic importance when compared with a reference plant, reference plant element, or reference population. For example, biotic stress tolerance may be shown by decreased pathogen load of tissues, decreased area of chlorotic tissue, decreased necrosis, improved growth, increased survival, increased biomass, increased shoot height, increased root length, etc. relative to a reference.

In some embodiments, the invention provides methods of improving plant health, wherein the method comprises heterologously disposing one or more endophytes to a plant element in an effective amount to improve a trait of agronomic importance in a plant derived from the treated plant element relative to a reference plant derived from a reference plant element, wherein the one or more endophytes comprises a first endophyte of the Order Coniochaetales, and a second endophyte of the Order Bacillales. In some embodiments, a method comprises heterologously disposing one or more endophytes to a plant element in an effective amount to improve a trait of agronomic importance in a plant derived from the treated plant element relative to a reference plant derived from a reference plant element, wherein the one or more endophytes comprises a first endophyte of the Family Coniochaetaceae, and a second endophyte of the Family Bacillaceae. In some embodiments, a method comprises heterologously disposing one or more endophytes to a plant element in an effective amount to improve a trait of agronomic importance in a plant derived from the treated plant element relative to a reference plant derived from a reference plant element, wherein the one or more endophytes comprises a first endophyte of the Genus Coniochaeta, and a second endophyte of the Genus Bacillus. In some embodiments, a method comprises heterologously disposing one or more endophytes to a plant element in an effective amount to improve a trait of agronomic importance in a plant derived from the treated plant element relative to a reference plant derived from a reference plant element, wherein the one or more endophytes comprises a first endophyte having at least one polynucleotide sequence that is at least 97% identical to SEQ ID NO. 29-37, or 38, and a second endophyte having at least one polynucleotide sequence that is at least 97% identical to SEQ ID NO. 39. In some embodiments, a method comprises heterologously disposing one or more endophytes to a plant element in an effective amount to improve a trait of agronomic importance in a plant derived from the treated plant element relative to a reference plant derived from a reference plant element, wherein the one or more endophytes comprises a first endophyte having at least one polynucleotide sequence that is at least 99% identical to SEQ ID NO. 29-37, or 38, and a second endophyte having at least one polynucleotide sequence that is at least 99% identical to SEQ ID NO. 39. In some embodiments, a method comprises heterologously disposing one or more endophytes to a plant element in an effective amount to improve a trait of agronomic importance in a plant derived from the treated plant element relative to a reference plant derived from a reference plant element, wherein the one or more endophytes comprises a first endophyte comprising the polynucleotide sequence SEQ ID NO. 29, and a second endophyte comprising the polynucleotide sequence SEQ ID NO. 39.

In some embodiments, the method comprises heterologously disposing one or more endophytes to a plant element, wherein the plant element is a seed (for example, a corn seed, a wheat seed, a rice seed, a soybean seed, etc.). In some embodiments, the method comprises heterologously disposing one or more endophytes to a plant element, wherein the plant element is wheat (for example, a wheat seed), and the trait of agronomic importance is one of more of increased root length, increased yield, and increased shoot length. In some embodiments, the method comprises heterologously disposing one or more endophytes to a plant element, wherein the plant element is corn, and the trait of agronomic importance is one of more of total grain weight, nutrition enhancement, shoot fresh weight, increased yield, increased win rate, and improved grain fill. In some embodiments, the method comprises heterologously disposing one or more endophytes to a plant element, wherein the plant element is rice, and the trait of agronomic importance is one or more of increased shoot dry weight, and tiller number. In some embodiments, the method comprises heterologously disposing one or more endophytes to a plant element, wherein the trait of agronomic importance is nutrition enhancement, and the nutrition enhancement comprises an increase in magnesium (Mg) or sulfur (S).

### Methods for Measuring Plant Health

The present invention includes methods of measuring plant health, comprising determining the presence or abundance of one or more endophytes in a plant element, growth medium and or growth environment. In some embodiments, the abundance or presence of the one or more endophytes in a plant element in an effective amount to improve a trait of agronomic importance is an indicator of plant health. In some embodiments, the abundance or presence of the one or more endophytes in a growth medium and or growth environment in an effective amount to improve a trait of agronomic importance of a plant element grown in the growth environment or growth medium may be used as a measure or predictor of plant health in a plant grown in that growth environment or growth medium. In some embodiments, the presence or abundance of one or more endophytes in a plant element, growth medium or growth environment can be detected before an improvement of a trait of agronomic importance can otherwise be observed or detected. In some embodiments, the presence or abundance of one or more endophytes is determined by polymerase chain reaction, fluorescence in situ hybridization, or isothermal amplification.

### Nucleic Acid Probes and Detection Kits

The present invention includes one or more nucleic acid probes that are markers of improved plant health. These probes include single and double stranded nucleic acids, engineered polymers such as peptide nucleic acids, or combinations thereof. In some embodiments, there are a plurality of nucleic acid probes. In some embodiments, the nucleic acid probes are attached to one or more solid supports. In some embodiments, the nucleic acid probes are reversibly attached to one or more solid supports. In some embodiments, the nucleic acid probes are attached to a contiguous solid support. In some embodiments, the nucleic acid probes are attached to a plurality of particles, for example beads. In some embodiments, only one unique sequence is attached to each particle. In some embodiments, nucleic acid probes attached to a solid support are physically separated from non-identical probes by an indentation or raised portion of the solid support. In some embodiments, the invention described herein provides a nucleic acid detection kit comprising any of the plurality of nucleic acid probes described herein.

In some embodiments, the one or more nucleic acid probes of the present invention may comprise sequences complementary or reverse complementary to one or more of SEQ ID NOs. 29-38. In some embodiments, the one or more nucleic acid probes of the present invention may comprise nucleic acid sequences complementary or reverse complementary to a nucleic acid sequence that is at least 97% identical to one or more of SEQ ID NOs. 29-38. In some embodiments, the one or more nucleic acid probes of the present invention may comprise sequences complementary or reverse complementary to the entire length of one or more of SEQ ID NOs. 29-38. In some embodiments, the one or more nucleic acid probes of the present invention may comprise sequences complementary or reverse complementary to a region within one or more of SEQ ID NOs. 29-38. In some embodiments, the region to which the nucleic acid probe is complementary or reverse complementary is a contiguous region. In some embodiments, the region to which the nucleic acid probe is complementary or reverse complementary is at least 5 nucleotides (nt) in length, at least 10 nt in length, at least 15 nt, between 10 nt and 30 nt, between 10 and 20 nt, between 15 and 50 nt, at least 20 nt, between 20 and 60 nt, at least 25 nt, at least 30 nt, at least 40 nt, at least 50 nt, between 50 nt and 100 nt, at least 60 nt, at least 70 nt, at least 80 nt, at least 100 nt in length. In some embodiments, the regions to which the nucleic acid probe is complementary or reverse complementary is not a contiguous region.

In some embodiments, a nucleic acid probe is capable of hybridizing to one or more of SEQ ID NOs. 29-38, or a reverse complement thereof. In some embodiments, the nucleic acid probe is capable of hybridizing under moderate conditions. “Moderate conditions” are 0.165M-0.330M NaCl and 20-29° C. below the melting temperature of the nucleic acid probe. In some embodiments, the nucleic acid probe is capable of hybridizing under stringent conditions. “Stringent conditions” are 0.0165M-0.0330M NaCl and 5-10° C. below the melting temperature of the nucleic acid probe.

In some embodiments, the nucleic acid probes are a component of a nucleic acid detection kit. In some embodiments, the nucleic acid probes are a component of a DNA detection kit. In some embodiments, the nucleic acid detection kit comprises additional reagents. In some embodiments, the contents of the nucleic acid detection kit are utilized in performing DNA sequencing.

In some embodiments, the one or more nucleic acid probes comprises at least 2, 3, 4, 5, 6, 7, 8, 9, or 10 probes.

The invention will be further described in the following examples, which do not limit the scope of the invention described in the claims.

### EXAMPLES

### Example 1. Isolation and Identification of Endophytes

### DNA Extraction

Endophytes of the present invention were isolated from the sources listed in Table 1.

TABLE 1
Sources of microbes of the present invention

| MIC ID | Isolated From | Isolation Environment |
|---|---|---|
| MIC-28421 | Pseudevernia | Arizona desert |
| | intensa (lichen) | |
| MIC-93265 | Zea mays | Agricultural cultivation |

Each sample was processed independently. Each sample was washed in a dilute water and detergent solution; tissue was collected from plants. Samples were surface sterilized by successive rinses: 2 minutes in 10% bleach solution, 2 minutes in 70% ethanol solution, and a rinse with sterile water. The series of rinses was repeated 3 times. The plant tissue was cut into small pieces with sterile scissors and blended with 3, 7 mm steel beads in 5-7.5 ml phosphate buffered solution (PBS). DNA was extracted from the ground tissues using the Magbind Plant DNA kit (Omega, Norcross, Georgia, USA) according to the manufacturer's instructions.

### Identification of Endophytes by Sequencing of Marker Genes

The endophytes were characterized by the sequences of genomic regions. Primers that amplify genomic regions of the endophytes of the present invention are listed in Table 2. IUPAC nucleotide ambiguity codes were used in the nucleic acid sequences of the present invention (N=any nucleotide, R=A or G, K=G or T, S=G or C, Y=C or T, M=A or C, W=A or T, B=not A, H=not G, D=not C, V=not T). Sanger sequencing was performed at Genewiz (South Plainfield, NJ). Raw chromatograms were converted to sequences, and corresponding quality scores were assigned using TraceTuner v3.0.6beta (U.S. Pat. No. 6,681,186). These sequences were quality filtered, aligned and a consensus sequence generated using Geneious v 8.1.8 (Biomatters Limited, Auckland NZ). The consensus sequences identifying the endophytes are listed in Table 3.

MIC-28421 was deposited with ______ as Deposit ID ______.

TABLE 2
Primer sequences useful in identifying microbes of the present invention

| Seq ID | Genomic Locus | Sequence |
|---|---|---|
| 1 | 60S ribosomal protein L 10 | GHGACAAGCGTTTCTCNGG |
| 2 | 60S ribosomal protein L 10 | CTTVAVYTGGAACTTGATGGT |
| 3 | ACT512f, Actin, primer-amplicon F | ATGTGCAAGGCCGGTTTCG |
| 4 | ACT783r, Actin, primer-amplicon R | TACGAGTCCTTCTGGCCCAT |
| 5 | Cmd5, Calmodulin | CCGAGTACAAGGARGCCTTC |
| 6 | Cmd6, Calmodulin | CCGATRGAGGTCATRACGTGG |
| 7 | GPD_128f, Glyceraldehyde-3-phosphate | TAAGTAGCCCCGAGACTGCT |
| | dehydrogenase | |
| 8 | EF_648r, Glyceraldehyde-3-phosphate | CTTCTGGGTGGCAGTGTAGG |
| | dehydrogenase | |
| 9 | ITS, Internal transcribed spacer | CTTGGTCATTTAGAGGAAGTAA |
| 10 | ITS, Internal transcribed spacer | TCCTGAGGGAAACTTCG |
| 11 | ITS, Internal transcribed spacer | GCTGCGTTCTTCATCGATGC |
| 12 | ITS, Internal transcribed spacer | GCATCGATGAAGAACGCAGC |
| 13 | ITS, Internal transcribed spacer | GTGARTCATCGAATCTTTG |
| 14 | ITS, Internal transcribed spacer | TCCTCCGCTTATTGATATGC |
| 15 | Phosphoglycerate kinase | GTYGAYTTCAAYGTYCC |
| 16 | Phosphoglycerate kinase | ACACCDGGDGGRCCGTTCCA |
| 17 | RPB1-Af, largest subunit of RNA | GARTGYCCDGGDCAYTTYGG |
| | polymerase II, primer-amplicon F | |
| 18 | RPB1-Cr, largest subunit of RNA | CCNGCDATNTCRTTRTCCATRTA |
| | polymerase II, primer-amplicon R | |
| 19 | bRPB2-7.1R, second largest subunit of | CCCATRGCYTGYTTMCCCATDGC |
| | RNA polymerase II, primer-amplicon R | |
| 20 | 5F_Eur, Second largest subunit of RNA | GAYGAYCGKGAYCAYTTCGG |
| | polymerase II | |
| 21 | 7CR_Eur, Second largest subunit of RNA | CCCATRGCYTGYTTRCCCAT |
| | polymerase II | |
| 22 | bRPB2-7.1R, Second largest subunit of | CCCATRGCYTGYTTMCCCATDGC |
| | RNA polymerase II | |
| 23 | fRPB2-5F, Second largest subunit of RNA | GAYGAYMGWGATCAYTTYGG |
| | polymerase II | |
| 24 | fRPB2-7R, Second largest subunit of RNA | CCCATWGCYTGCTTMCCCAT |
| | polymerase II | |
| 25 | TEF1, Eff, Translation elongation factor | TACCATCGAGAAGTTCGAGA |
| 26 | TEF1, Efr, Translation elongation factor | CATCCTTGGAGATACCAGCC |
| 27 | Btub2Fd, beta-tubulin, primer-amplicon F | GTBCACCTYCARACCGGYCARTG |
| 28 | Btub4Rd, beta-tubulin, primer-amplicon R | CCRGAYTGRCCRAARACRAAGTTGTC |

TABLE 3
Exemplary sequences of endophytes of the present invention

| SEQ | MIC | Sequence | |
|---|---|---|---|
| ID | ID | Description | Sequence |
| 29 | MIC-28421 | Internal | CAAGAAGCCGAAAGGCTACTTCAAACCATTGCGAACTCGTCCAAGTT |
| | | transcribed | GCTTCGGCGGCGCGGCACCCCCTAACGGGGGCGCCGCAGCCCTGCCTC |
| | | spacer (ITS) | TCCGGAGGTTCGGGGCGCCCGCCGGAGGTACGAAACTCTGTATTATAG |
| | | | TGGCATCTCTGAGTATAAAGCAAATAAGTTAAAACTTTCAACAACGGA |
| | | | TCTCTTGGCTCTGGCATCGATGAAGAACGCAGCGAAATGCGATAAGTA |
| | | | ATGTGAATTGCAGAATTCAGTGAATCATCGAATCTTTGAACGCACATT |
| | | | GCGCCCGGTAGTACTCTACCGGGCATGCCTGTTCGAGCGTCATTTCAA |
| | | | CCCTCAAGCTCTGCTTGGTGTTGGGGCCCTACGGCTGCCGTAGGCCCT |
| | | | GAAAGGAAGTGGCGGGCTCGCTACAACTCCGAGCGTAGTAATTCATT |
| | | | ATCTCGCTAGGGAGGTTGCGGCGTGCTCCTGCCGTTAAAGACCCATCT |
| | | | TTAACCA |
| 30 | MIC-28421 | 60S | CTTCAGCTGGAACTTGATGGTCGACTTGACCTCGGTGATCTTGTTGGA |
| | | ribosomal | GAGGTCATCGGCGTGGGACACGGGGGTCGGGAACTTGCCGGCCTTGG |
| | | protein L 10 | AGAGACCGGGACCGAGCAGACGGGGGATCTGCTTGATCAGGGTGTCG |
| | | | GAGGCGATGAAAGCGGCGTACTTGCGGGCCAGCTTCTTGATCACTGTT |
| | | | GTATCGGTTAGTATCGAGGTCAAAGACCAGGAAGGCGAACCGGTTGC |
| | | | CACATACGCTTCTTGTTCTTGTTCAGCTTCTTCAAGTCGTCGGCCGACA |
| | | | TGGAGTCGACACCGCCGTGCTTCGCACGATCGATATCGTGCTGGTCAC |
| | | | CGAGAATGCAGATGGCCATGTTCGGGCGGGGAACAGTGGGCAGCTTG |
| | | | ACGGTGCCAGAGAAACGCTTGTCACG |
| 31 | MIC-28421 | Actin- | CCGGTATCGTGCTCGACTCCGGTGACGGTGTCACCCACGTTGTGCCCA |
| | | partial | TCTACGAGGGTTTCGCTCTTCCCCACGCCATTGCCCGTGTCGACATGG |
| | | (ACT) | CTGGCCGTGACTTGACCGACTACCTCATGAAGATCCTTGCTGAGCGCG |
| | | | GTTACGCCTTCTCCACCACTGCCGAGCGCGAAATCGTTCGTGACATCA |
| | | | AGGAGAAGCTCTGCTACGTTGCCCTCGACTTCGAGCAGGAGATCCAG |
| | | | ACCGCCGCCCAGAGCTCCAGCTTGGAGAAGTCGTATGAGCTTCCCGAC |
| | | | GGTCAGGTCATCACCCTCGGCAACGAGCGCTTCCGTGCTCCTGAGGCT |
| | | | CTCTTCCAGCCTTCTGTCCTCGGTCTCGAGAGCGGTGGCATCCACGTC |
| | | | ACCACTTTCAACTCGATCATGAAGTGCGACGTCGATGTCCGGAAGGAT |
| | | | CTCTATGGTAACATCGTTATGGTACGTTCTGCCTCTTATGGCTCCTCAA |
| | | | AGCGAAAACAATGGAAGAATATAGCTAACAGCGCACTAGTCTGGTGG |
| | | | CACCACCATGTACCCCGGTCTC |
| 32 | MIC-28421 | Calmodulin- | GGTCGAAAACCTGTTGATATCGGTTAGCACGTGTGCTATATCTGTTAT |
| | | partial | AAAAAATGTGATTGTATGTATCTTGCCTTGAAAGCTTCTCGAATCTCTT |
| | | (CMD) | CCTCGGAATCAGTATCCTTCATCTTCCGGGCCATCATGGTAAGAAACT |
| | | | CTGGCAATCGTCAGCGCCGCGACCTCCACACGTGTACTTTTCAAGTGA |
| | | | TAGACCTACCGGGGAAGTCAATGGTGCCGTTGTTGTCGGCATCAACCT |
| | | | CGTTGATCATGTCTTGGAGCTCGGACTCGGAGGGGTTCTGTCCTAGAG |
| | | | AGCGCATGACCGTTCCCAGTTCCTTGGTAGTGATTTGTCCTTGGTGTAA |
| | | | GTCAGCAATGTTGTTACTCGAGAAATGAGATCGAGTGGTAAGGCAGT |
| | | | AGCAGCGACACAGCGCCTGCGGATCTATCGACGCCGTAGAATTGATC |
| | | | GCGCATCCGCATTTGCCGCAGAGAGATAAAGGTAGGGCAGTACTAAC |
| | | | CATCGCCATCTTTGTCCTGAACATGCGCCCGTCAGCAATAGTCACACG |
| | | | CGCCGAACAAGACGC |
| 33 | MIC-28421 | Glyceraldehyde- | AACGAGGAGACCTACGACGGCAAGGCCGACGTCATCTCCAACGCCTC |
| | | 3-phosphate | GTGCACCACCAACTGCCTTGCCCCCCTCGCCAAGGTCGTCAACGACAA |
| | | dehydrogenase | GTTCACCATCATTGAGGGTCTGATGACCACCATCCACTCCTACACCGC |
| | | (GPD) | CACCCAGAAGACCGTCGACGGTCCTTCGTCCAAGGACTGGCGCGGTG |
| | | | GCCGTGGTGCTGCCCAGAACATCATCCCCAGCAGCACCGGTGCCGCCA |
| | | | AGGCCGTCGGCAAGGTCATCCCCGAGCTCAACGGCAAGCTGACCGGC |
| | | | ATGTCGATGCGTGTCCCCACCTCCAACGTCTCCGTTGTCGATCTTACCT |
| | | | GCCGTATCGAGAAGCCTGCTTCGTACGACGAGATCAAGGCCGCCATC |
| | | | AAGGAGGCCGCTGAGGGTCCCCTCAAGGGTATGTCTCATTCTCCTCAA |
| | | | CCCCTAAATGAAACCAGAACAGAAAAGCTAACCTGTCCCAGGAGTCC |
| | | | TTGCCTACACCGAGGACGAGGTCGTCTCCTCCGACATGAACGGCA |
| 34 | MIC-28421 | Phosphoglycerate | ACACCGGCGGGGCCGTTCCACAGGATGGTCTGCGCGTCGCCGATGGC |
| | | kinase (PGK) | CTCCTCGTACAGCTTGATCGACTCCGGGCCGCAGTCGAGGCCCATCCA |
| | | | GCCGTCGGGGATGCCGTCCTTGTCCGTGGCGGTGCCAGTCTGCGCGTC |
| | | | CTTGTCGAACTTGTCGGCGATAACGTAGTCGACGGGGAGCACGACCTT |
| | | | GACGTTGTTGGCCTTGGCCTTCTCCATGAGCTTGCCGACCGTCTTGGCG |
| | | | CCGGCCTCGTCGAAGAGCGAGGTGCCGATGGGGATGTTGTCCAGCGT |
| | | | CTTCTTGAAGGTGAAGGCCATGCCGCCGCAGATGACGAGCGTGTCCAC |
| | | | CTTCTCGAGCAGGTTGTCGATCAGCTGGATCTTGTCGCTGACCTTGGC |
| | | | GCCGCCCAGGATGGCCAGGAAGGGCCGCTTGGGGTTCTCGAGGGCCT |
| | | | TGGCGAAGTAGTCGAGCTCCTTCTTCATCAGGTAGCCGGCGGCCTTTT |
| | | | GTGGGAGGTCGATGCCGACCATGGAGGAGTGGGCGCGGTGTGCGGTG |
| | | | CCGAAGGCATCGTCTGAAGTGCGGTTGTGTTAGTTTCAGGGGCGCATA |
| | | | CTAGAGCTGCTGTCTGACAAAACTTACTGACGTAAACGTCCCCGAGCT |
| | | | TGGTGAGTCCCTTGCGGAACTCCTCGACCTTGGCCTTGTCGGCCTTGA |
| | | | CCTTGTTGCCCTCGGCATCAACGCCCTTGCCCTCCTCCTCGATGTGGAA |
| | | | GCGGAGGTTCTCCAGCAGCACAACGTCGCCATTGTCCGCCTTGGAGAC |
| | | | GATCTCCTCGACCTCGGGGCCGACGCAGTCCGGCGCGAAGGTGACCTT |
| | | | CTTGCCCAGCAGCTTCTCGAGCTCGGGCACGACGGGCTTGAGCGAGA |
| | | | ACTTCTCCTTGACCTGGCCGTCGGGGCGGCCGAGGTGGGACATGAGG |
| | | | ATCACGGCCTTGGCGCCGTTGTCGATGGCATACTTGATTGTGGGGATG |
| | | | GCGCCGGCAATGCGCTGGTTGTTGGTGACCTTCTTGTTCGCGTCGAGG |
| | | | GGGACGTTGAAGTCAACC |
| 35 | MIC-28421 | Largest | CCAGCAATATCGTTGTCCATGTACGTGGCAACGTGATACTGCAGCAGA |
| | | subunit of | TCCTCGAAATCCGTGATAATGTGCTGAGGCGCACCATCCGCGATAGCT |
| | | RNA | TGCCTAACATTGCTGTTGGCTCGAATGATGTCACCAAGCTTGTATGTC |
| | | polymerase | AAATCGTCCTCGTTACGCAGGCCTGTCCCGCTGCCGTCCATGGAAATG |
| | | II (RPB1) | CTAGGCCGAACAGGAGGTGGCGGCACGGGAAGAACGGTGACAATCA |
| | | | ACCACTCTGGGCGAGCATAGTCAACGTTGAGGCCCATATCACGAAGG |
| | | | TCCGAATCCGAGATCCGGCGAAGAATGCCGTGAGCCATCTCTGGTGTC |
| | | | AAAACCGTTTTGTCTTTGACGGTAGCACCATCCTCGGTCTTCGAGCTCT |
| | | | TCGTCGTAAACAGCTGAAGAGCTGCCTGGCGAACCGTCGGCTGCTGGT |
| | | | TTCCACATCCACCATGCTTGATTGTCGGCTTAGCATTCGGATCATTGGA |
| | | | ACCAAATTCGGCATCCTTGTCCTGCTTGACATCCTTCTCTATGTAGCAC |
| | | | ATGGTCTGCTTCTTGCAAACAGCCCACACGCGGTTGAAGCGAAGTTTG |
| | | | GGGTCTCTGATATTGACGGCGGCGAGAAATTCATCATCAGACTAGAGC |
| | | | GGAGTGCAAGATTAGCATCAAGAAGATCATGCAAAGCAACCAAAGCA |
| | | | GTGCAGACTAACCCTGTCAGCCAACACCTTGCTACAGTTATGACAGAC |
| | | | GATCTCGAGGATCTTCTTGACTTTCTTGATGAAGCCAGGGTGGTAGAC |
| | | | GGGTTTAGCCAACTCGATGTGACCAAAATGGCCCGGACACTCA |
| 36 | MIC-28421 | Second | CCCATGGCTTGCTTGCCCATGGCAGACTGATAGGTGTTTCGGGGCGAC |
| | | largest | TGGTTGTGGTCAGGGAACGGGATAATGCTGGCGCAGATGCCCAGGAG |
| | | subunit of | CATGCTGGGGTGGATTTCGCAGTGAGTGTAGGCGTGTGTGGTGGGATT |
| | | RNA | TATCCGGGTCTTGATGCGCGCGTTGGGCTCATTCTCGTGGTTGTCCGG |
| | | polymerase | AATCTTGAAGCCAGCCTTTTGTGCTCGGAAGTCGGCAAGATCTTCCGG |
| | | II (RPB2) | CGTCATACATATCATGGATGTCTCTTCCTCCTCAGCGTCGAGGTAGTC |
| | | | GATGCAGCCTGCGTCCTCCAACCCCTTGAAGCCAAAGTACTCTGGGTC |
| | | | GTCCTTGGCTAATTGAGCGTCGCGTCTGAGCTTCTCAATGTGCGTCTG |
| | | | GTTGATGACCAGCTCGCCCACAGGTGTTTCCACGTTCTCCCGTGTCTCA |
| | | | ACGACGAACAGCGGTCTCATCACACGTCCCGCATCGGAGAAGATCTT |
| | | | GAATTCTCGGTCTCGAATATCTCGGACCAGCGAGACCTCGGGCGGTAT |
| | | | GTCACCCTCACGACGGAGGTTTGTGATTTGAGCCACAAGATTCTTCGG |
| | | | ATCCTGGTGCACGCCGACCCATGTGCCGTTGACAAAAACCTTGGTTGC |
| | | | GTTCGGGAACCGAAGGGGCTCGTACTCCTCCAACACCTCCATATTCCT |
| | | | TGCAATCATGAAGTCGATAAGCGGCTCAGCGGGTTGCCCCACGCTCAC |
| | | | GTAGCACATGAGGGCCAGATTCTTGACAAGGCCACAAGCTTGCCCCTC |
| | | | GGGGGTCTCAGCAGGGCAGACAAGACCCCAATGTGTGTTGTGCAGTT |
| | | | GTCGTGGCTTTGCTAACTTGCCGTCACGGCCAATTGGCGTGTTGGTGC |
| | | | GTCTCAAATGCGACAGTGTGGAAGCGAACGTATAACGGTTCAGCACTT |
| | | | GCGAGACACCAGCGGTGGTGCTCATTGCCTTCTTCTGGTCGCCCCAGT |
| | | | TTCCTGTCGCAAGAGAGTACTTCAGAGCGTTGGTGATGGTGGACGGGT |
| | | | GGATGCCCTGGACCAACGAGAACTTCTTGTCTTGGTCCACGCACTTCT |
| | | | TCAAGTAGCTCATGAGGTCCTGCGTCATTCTGCGAACGATGTTTCGAA |
| | | | AAAGCTTCGCCAGCAGAGGACCGGCCAGGTCGAGGCGCTTCTTGCCA |
| | | | AAGTGGTCACGGTCGTCG |
| 37 | MIC-28421 | Translation | CTCATCATTGCCGCTGGTACTGGTGAGTTCGAGGCTGGTATCTCCAAG |
| | | elongation | GATGGCCAGACTCGTGAGCACGCTCTGCTCGCCTTCACCCTCGGTGTC |
| | | factor | AGGCAGCTCATCGTCGCCATCAACAAGATGGACACCGCCAAGTGGTC |
| | | (TEF1) | TGAGGATCGTTACAACGAGATCATCAAGGAGACCTCTTCCTTCATCAA |
| | | | GAAGGTCGGCTACAACCCCAAGCAGGTCGCTTTCGTCCCCATCTCGGG |
| | | | TTTCAACGGCGACAACATGCTGGCCGCCTCCACCAACTGCCCCTGGTA |
| | | | CAAGGGCTGGAAGAAGGAGGGCAAGAACGGTGAGATCACCGGCAAG |
| | | | ACCCTCCTCGAGGCCATCGACTCTTGCGAGCCCCCCAAGCGTCCTACC |
| | | | GACAAGCCCCTCCGTCTTCCCCTCCAGGATGTTTACAAGATCGGCGGC |
| | | | ATTGGAACAGTGCCCGTCGGCCGTATCGAGACTGGAAAGCTCATTCCC |
| | | | GGTATGGTCGTTACCTTCGCTCCCGCCAACGTCACCACTGAAGTCAAG |
| | | | TCCGTCGAGATGCACCACGAGCAGCTCAAGGAGGGTCTCCCCGGTGA |
| | | | CAACGTCGGCTTCAACGTGAAGAACGTCTCCGTCAAGGAGATCCGTCG |
| | | | TGGCAACGTCGCTGGTGACAGCAAGAACGACCCCCCCATGGGCGCCG |
| | | | CTTCTTTCCTCGCCCAGGTCATCGTCCTGAACCACCCCGGTCAGGTCG |
| | | | GTGCTGGATACGCCCCGGTTCTCGACTGCCACACTGCCCACATTGCCT |
| | | | GCAAGTTCGCCGAGATCCAGGAGAAGATTGACCGCCGTACCGGAAAG |
| | | | TCGACTGAGTCCAACCCCAAGTTCATCAAGTCGGGTGACGCTGCCATC |
| | | | GTCAAGATGATTC |
| 38 | MIC-28421 | Beta tubulin | CCCGACTGGCCGAAAACGAAGTTGTCGGGACGGAAGAGCTGACCGAA |
| | | 2 (TUB2) | GGGACCAGCGCGGACAGCGTCCATGGTACCCGGTTCGAGATCGACGA |
| | | | GGACAGCGCGGGGAACATACTTGTTGCCGGAAGCCTAGTTTTGTGATT |
| | | | ACGAGTCAATAGCTGCGGTATGGAGCCATGGCTTGATGACTGGATGG |
| | | | ATAGCTTACCTCGTTGAAGTAGACGCTCATACGCTCGAGCTGGAGCTC |
| | | | CGAAGTGCCGTTGTACCTAGATGGCGGTGAGCTTGAGCGGTACGAAG |
| | | | ATCACGTTAGAGCTGTTACGTACACGCCATTGCTGTCGAGGCCGTGCT |
| | | | CGCCGGAGATAGTTTGCCTGTTATCGTTATTAGAATCTCAGTTCGAAC |
| | | | GCAGGTCTCAACGTCGAGTTGTTGGTCTGAAGGCGAACGCACCAGAA |
| | | | GGCAGCACCGATTTGGTTACCCTGCGAGACATGTTAGCAGGCCATCAC |
| | | | TCAAGCACGAGAAGATAGACACGACTTACGCATTGACCGGTCTGAAG |
| | | | ATGGACC |
| 39 | MIC-93265 | 16S | GGCTCAGGACGAACGCTGGCGGCGTGCCTAATACATGCAAGTCGAGC |
| | | | GAATCGATGGGAGCTTGCTCCCTGAGATTAGCGGCGGACGGGTGAGT |
| | | | AACACGTGGGCAACCTGCCTATAAGACTGGGATAACTTCGGGAAACC |
| | | | GGAGCTAATACCGGATACGTTCTTTTCTCGCATGAGAGAAGATGGAAA |
| | | | GACGGTTTACGCTGTCACTTATAGATGGGCCCGCGGCGCATTAGCTAG |
| | | | TTGGTGAGGTAATGGCTCACCAAGGCGACGATGCGTAGCCGACCTGA |
| | | | GAGGGTGATCGGCCACACTGGGACTGAGACACGGCCCAGACTCCTAC |
| | | | GGGAGGCAGCAGTAGGGAATCTTCCGCAATGGACGAAAGTCTGACGG |
| | | | AGCAACGCCGCGTGAACGAAGAAGGCCTTCGGGTCGTAAAGTTCTGT |
| | | | TGTTAGGGAAGAACAAGTACCAGAGTAACTGCTGGTACCTTGACGGT |
| | | | ACCTAACCAGAAAGCCACGGCTAACTACGTGCCAGCAGCCGCGGTAA |
| | | | TACGTAGGTGGCAAGCGTTGTCCGGAATTATTGGGCGTAAAGCGCGC |
| | | | GCAGGTGGTTCCTTAAGTCTGATGTGAAAGCCCACGGCTCAACCGTGG |
| | | | AGGGTCATTGGAAACTGGGGAACTTGAGTGCAGAAGAGGAAAGTGGA |
| | | | ATTCCAAGTGTAGCGGTGAAATGCGTAGAGATTTGGAGGAACACCAG |
| | | | TGGCGAAGGCGACTTTCTGGTCTGTAACTGACACTGAGGCGCGAAAG |
| | | | CGTGGGGAGCAAACAGGATTAGATACCCTGGTAGTCCACGCCGTAAA |
| | | | CGATGAGTGCTAAGTGTTAGAGGGTTTCCGCCCTTTAGTGCTGCAGCT |
| | | | AACGCATTAAGCACTCCGCCTGGGGAGTACGGCCGCAAGGCTGAAAC |
| | | | TCAAAGGAATTGACGGGGGCCCGCACAAGCGGTGGAGCATGTGGTTT |
| | | | AATTCGAAGCAACGCGAAGAACCTTACCAGGTCTTGACATCCTCTGAC |
| | | | AACCCTAGAGATAGGGCTTTCCCCTTCGGGGGACAGAGTGACAGGTG |
| | | | GTGCATGGTTGTCGTCAGCTCGTGTCGTGAGATGTTGGGTTAAGTCCC |
| | | | GCAACGAGCGCAACCCTTGATCTTAGTTGCCAGCATTCAGTTGGGCAC |
| | | | TCTAAGGTGACTGCCGGTGACAAACCGGAGGAAGGTGGGGATGACGT |
| | | | CAAATCATCATGCCCCTTATGACCTGGGCTACACACGTGCTACAATGG |
| | | | ATGGTACAAAGGGCTGCAAACCTGCGAAGGTAAGCGAATCCCATAAA |
| | | | GCCATTCTCAGTTCGGATTGCAGGCTGCAACTCGCCTGCATGAAGCCG |
| | | | GAATCGCTAGTAATCGCGGATCAGCATGCCGCGGTGAATACGTTCCCG |
| | | | GGCCTTGTACACACCGCCCGTCACACCACGAGAGTTTGTAACACCCGA |
| | | | AGTCGGTGAGGTAACCTTCATGGAGCCAGCCGCCTAAGGTGGGACAG |
| | | | ATGATTGGGGT |

### Example 2. Taxonomic Classification of Endophytes

Classification of the fungal strain using ITS sequences was done by the following methodology.

Total genomic DNA was extracted from individual fungal isolates, using the DNeasy Plant Mini Kit (Qiagen, Germantown, MD). Polymerase Chain Reaction (PCR) was used to amplify a genomic region including the nuclear ribosomal internal transcribed spacers (ITS) using a primer pair ITS_1 (5′-CTTGGTCATTTAGAGGAAGTAA-3′) (SEQ ID NO: 9) and LR5 (5′-TCCTGAGGGAAACTTCG-3′) (SEQ ID NO: 10). Each 25 microliter-reaction mixture included 22.5 microliters of Invitrogen Platinum Taq supermix, 0.5 microliter of each primer (10 uM), and 1.5 microliters of DNA template (˜2-4 ng). Cycling reactions were run with MJ Research PTC thermocyclers and consisted of 94° C. for 5 min, 35 cycles of 94° C. for 30 s, 54° C. for 30 s, and 72° C. for 1 min, and 72° C. for 10 min. Sanger sequencing of was performed at Genewiz (South Plainfield, NJ) using primers: ITS_1 (5′-CTTGGTCATTTAGAGGAAGTAA-3′) (SEQ ID NO: 9), ITS_2 (5′-GCTGCGTTCTTCATCGATGC-3′) (SEQ ID NO: 11), ITS_3 (5′-GCATCGATGAAGAACGCAGC-3′) (SEQ ID NO: 12), and LR5 (5′-TCCTGAGGGAAACTTCG-3′) (SEQ ID NO: 10). Sequencing primers were chosen so that overlapping regions were sequenced. Raw chromatograms were converted to sequences, and corresponding quality scores were assigned using TraceTuner v3.0.6beta (U.S. Pat. No. 6,681,186). These sequences were quality filtered, aligned and a consensus sequence generated using Geneious v 8.1.8 (Biomatters Limited, Auckland NZ).

Taxonomic classifications were assigned to the sequences using the highest probability of assignment based on the results of industry standard taxonomic classification tools: LCA (runs USEARCH (Edgar, R. C. (2010) Bioinformatics. 26(19):2460-2461) with option search_global, then for all best match hits, returns lowest taxonomic rank shared by all best hits for a query), SPINGO (Allard et al. (2015) BMC Bioinformatics. 16: 324), and UTAX (Edgar, R. C., 2016), using the WARCUP Fungal ITS trainset 1 (Deshpande et al. (2016) Mycologia 108(1):1-5) and UNITE (Koljalg et al. (2013) Molecular Ecology, 22: 5271-5277). The classifier and database combinations listed in Table 4 were used to assign taxonomy to fungal sequences.

TABLE 4
The classifier and database combinations used to classify ITS sequences

| Classifier | Database |
|---|---|
| LCA | UNITE, Fungal ITS trainset Jul. 4, 2014 |
| RDP | UNITE, Fungal ITS trainset Jul. 4, 2014; |
| | WARCUP, Fungal ITS trainset 1 |
| SPINGO | UNITE, Fungal ITS trainset Jul. 4, 2014 |
| UTAX | UNITE, Fungal ITS trainset Jul. 4, 2014; |
| | WARCUP, Fungal ITS trainset 1 |

TABLE 4
The classifier and database combinations
used to classify ITS sequences

| Classifier | Database |
|---|---|
| LCA | UNITE, Fungal ITS trainset Jul. 4, 2014 |
| RDP | UNITE, Fungal ITS trainset Jul. 4, 2014; |
| | WARCUP, Fungal ITS trainset 1 |
| SPINGO | UNITE, Fungal ITS trainset Jul. 4, 2014 |
| UTAX | UNITE, Fungal ITS trainset Jul. 4, 2014; |
| | WARCUP, Fungal ITS trainset 1 |

### Example 3: Assessment of Improved Plant Characteristics: Vigor Assay

### Assay of Soy Seedling Vigor

Seed preparation: The lot quality of soybean seeds was first assessed by testing germination of 100 seeds. Seeds were placed, 8 seeds per petri dish, on filter paper in petri dishes, 12 ml of water was added to each plate and plates are incubated for 3 days at 24° C. The process would have been repeated with a fresh seed lot if fewer than 95% of the seeds had germinated. One thousand soybean seeds were then surface sterilized by co-incubation with chlorine gas in a 20×30 cm container placed in a chemical fume hood for 16 hours. Percent germination of 50 seeds, per sterilization batch, was tested as above and confirmed to be greater than 95%.

Preparation of endophyte treatments: Spore solutions were made by rinsing and scraping spores from agar slants which had been growing for about 1 month. Rinsing was done with 0.05% Silwet. Solutions were passed through Miracloth to filter out mycelia. Spores per ml were counted under a microscope using a hemocytometer. The stock suspension was then diluted into 10\hat{ }6 spores/ml utilizing water. 3 μl of spore suspension was used per soy seed (˜10\hat{ }13 CFUs/seed is obtained). Control treatments were prepared by adding equivalent volumes of sterile water to seeds.

Assay of seedling vigor: Two rolled pieces of germination paper were placed in a sterile glass jar with 50 ml sterile water, then removed when completely saturated. Then the papers were separated and inoculated seeds were placed at approximately 1 cm intervals along the length of one sheet of moistened germination paper, at least 2.5 cm from the top of the paper and 3.8 cm from the edge of the paper. The second sheet of paper was placed on top of the soy seeds and the layered papers and seeds were loosely rolled into a tube. Each tube was secured with a rubber band around the middle and placed in a single sterile glass jar and covered loosely with a lid. For each treatment, three jars with 15 seeds per jar were prepared. The position of jars within the growth chamber was randomized. Jars were incubated at 60% relative humidity, and 22° C. day, 18° C. night with 12 hours light and 12 hours dark for 4 days and then the lids were removed and the jars were incubated for an additional 7 days. Then the germinated soy seedlings were weighed and photographed, and root length, root area, and seedling fresh weight were scored as follows.

Dirt, excess water, seed coats and other debris were removed from seedlings to allow accurate scanning of the roots. Individual seedlings were laid out on clear plastic trays and trays were arranged on an Epson Expression 11000XL scanner (Epson America, Inc., Long Beach CA). Roots were manually arranged to reduce the amount of overlap. For root measurements, shoots were removed if the shape of the shoot caused it to overlap the roots.

The WinRHIZO software version Arabidopsis Pro2016a (Regents Instruments, Quebec Canada) was used with the following acquisition settings: greyscale 4000 dpi image, speed priority, overlapping (1 object), Root Morphology: Precision (standard), Crossing Detection (normal). The scanning area was set to the maximum scanner area. When the scan was completed, the root area was selected, and root length and root surface area were measured.

Statistical analysis was performed using R (R Core Team, 2016. R: A language and environment for statistical computing. R Foundation for Statistical Computing, Vienna, Austria. R-project.org/). Improvement of plant health is shown by percent change of one or more metrics relative to controls (e.g., 10% means microbe performed 10% above control performance; −10% means microbe performed 10% below control performance). Assays were run two times; results are shown in the following tables.

TABLE 6
Plant vigor of endophyte and control treated soy seedlings

| | | | % Delta relative |
|---|---|---|---|
| MIC-ID | Crop | Metric | to Control |
| MIC-28421 | Soybeans | Root Length | −21 |
| MIC-28421 | Soybeans | Root Area | −15 |
| MIC-28421 | Soybeans | Seedling Fresh Weight | 1 |
| MIC-28421 | Soybeans | Root Area | −8 |
| MIC-28421 | Soybeans | Root Length | −11 |
| MIC-28421 | Soybeans | Seedling Fresh Weight | 2 |

### Assay of Rice Seedling Vigor

Seed preparation: The lot of rice seeds is first evaluated for germination by transfer of 100 seeds and with 8 ml of water to a filter paper lined petri dish. Seeds are incubated for 3 days at 24° C. The process is repeated with a fresh seed lot if fewer than 95% of the seeds have germinated. Rice seeds are then surface sterilized by co-incubation with chlorine gas in a 20×30 cm container in a chemical fume hood for 12 hours. Percent germination of 50 seeds, per sterilization batch, is tested as above and confirmed to be greater than 95%.

Optional reagent preparation: 7.5% polyethylene glycol (PEG) is prepared by adding 75 g of PEG to 1000 ml of water, then stirring on a warm hot plate until the PEG is fully dissolved. The solution is then autoclaved.

Preparation of endophyte treatments: Spore solutions are made by rinsing and scraping spores from agar slants which are grown for about 1 month. Rinsing is done with 0.05% Silwet. Solutions are passed through Miracloth to filter out mycelia. Spores per ml are counted under a microscope using a hemocytometer. The stock suspension is then diluted into 10\hat{ }6 spores/ml utilizing water. 3 μl of spore suspension is used per rice seed (˜10\hat{ }3 CFUs/seed is obtained). Seeds and spores are combined in a 50 ml falcon tube and gently shaken for 5-10 seconds until thoroughly coated. Control treatments are prepared by adding equivalent volumes of sterile water to seeds.

Assay of seedling vigor: Petri dishes are prepared by adding four sheets of sterile heavy weight seed germination paper, then adding either 50 ml of sterile water or, optionally, 50 ml of PEG solution as prepared above, to each plate then allowing the liquid to thoroughly soak into all sheets. The sheets are positioned and then creased so that the back of the plate and one side wall are covered, two sheets are then removed and placed on a sterile surface. Along the edge of the plate across from the covered side wall 15 inoculated rice seeds are placed evenly at least one inch from the top of the plate and half an inch from the sides. Seeds are placed smooth side up and with the pointed end of the seed pointing toward the side wall of the plate covered by germination paper. The seeds are then covered by the two reserved sheets, and the moist paper layers smoothed together to remove air bubbles and secure the seeds, and then the lid is replaced. For each treatment, at least three plates with 15 seeds per plate are prepared. The plates are then randomly distributed into stacks of 8-12 plates and a plate without seeds is placed on the top. The stacks are incubated at 60% relative humidity, and 22° C. day, 18° C. night with 12 hours light and 12 hours dark for 24 hours, then each plate is turned to a semi-vertical position with the side wall covered by paper at the bottom. The plates are incubated for an additional 5 days, then rice seeds are scored manually for germination, root and shoot length.

Statistical analysis is performed using R or a similar statistical software program.

### Assay of Corn Seedling Vigor

Seed preparation: The lot quality of corn seeds is first evaluated for germination by transfer of 100 seeds with 3.5 ml of water to a filter paper lined petri dish. Seeds are incubated for 3 days at 24° C. The process is repeated with a fresh seed lot if fewer than 95% of the seeds have germinated. One thousand corn seeds are then surface sterilized by co-incubation with chlorine gas in a 20×30 cm container in a chemical fume hood for 12 hours. Percent germination of 50 seeds, per sterilization batch, is tested as above and confirmed to be greater than 95%.

Optional reagent preparation: 7.5% PEG 6000 (Calbiochem, San Diego, CA) is prepared by adding 75 g of PEG to 1000 ml of water, then stirred on a warm hot plate until the PEG is fully dissolved. The solution is then autoclaved.

Preparation of endophyte treatments: Spore solutions are made by rinsing and scraping spores from agar slants which are grown for about 1 month. Rinsing is done with 0.05% Silwet. Solutions are passed through Miracloth to filter out mycelia. Spores per ml are counted under a microscope using a hemocytometer. The stock suspension is then diluted into 10\hat{ }6 spores/ml utilizing water. 3 μl of spore suspension is used per corn seed (˜10\hat{ }13 CFUs/seed is obtained). Control treatments are prepared by adding equivalent volumes of sterile water to seeds.

Assay of seedling vigor: Either 25 ml of sterile water or, optionally, 25 ml of PEG solution as prepared above, is added to each Cyg™ germination pouch (Mega International, Newport, MN) and place into pouch rack (Mega International, Newport, MN). Sterile forceps are used to place corn seeds prepared as above into every other perforation in the germination pouch. Seeds are fitted snugly into each perforation to ensure they do not shift when moving the pouches. Before and in between treatments forceps are sterilized using ethanol and flame and workspace wiped down with 70% ethanol. For each treatment, three pouches with 15 seeds per pouch are prepared. The germination racks with germination pouches are placed into plastic tubs and covered with perforated plastic wrap to prevent drying. Tubs are incubated at 60% relative humidity, and 22° C. day, 18° C. night with 12 hours light and 12 hours dark for 6 days to allow for germination and root length growth. Placement of pouches within racks and racks/tubs within the growth chamber is randomized to minimize positional effect. At the end of 6 days the corn seeds are scored manually for germination, root and shoot length.

Statistical analysis is performed using R or a similar statistical software program.

### Assay of Wheat Seedling Vigor

Seed preparation: The lot of wheat seeds was first evaluated for germination by transfer of 100 seeds and with 8 ml of water to a filter paper lined petri dish. Seeds were incubated for 3 days at 24° C. The process was repeated with a fresh seed lot if fewer than 95% of the seeds had germinated. Wheat seeds were then surface sterilized by co-incubation with chlorine gas in a 20×30 cm container in a chemical fume hood for 12 hours. Percent germination of 50 seeds, per sterilization batch, was tested as above and confirmed to be greater than 95%.

Optional reagent preparation: 7.5% polyethylene glycol (PEG) was prepared by adding 75 g of PEG to 1000 ml of water, then stirring on a warm hot plate until the PEG was fully dissolved. The solution was then autoclaved.

Preparation of endophyte treatments: Spore solutions were made by rinsing and scraping spores from agar slants which had been growing for about 1 month. Rinsing was done with 0.05% Silwet. Solutions were passed through Miracloth to filter out mycelia. Spores per ml were counted under a microscope using a hemocytometer. The stock suspension was then diluted into 10\hat{ }6 spores/ml utilizing water. 3 μl of spore suspension was used per wheat seed (˜10\hat{ }3 CFUs/seed was obtained). Seeds and spores were combined in a 50 ml falcon tube and gently shaken for 5-10 seconds until thoroughly coated. Control treatments were prepared by adding equivalent volumes of sterile water to seeds.

Assay of seedling vigor: Petri dishes were prepared by adding four sheets of sterile heavy weight seed germination paper, then 50 ml of sterile water (optionally, 50 ml of PEG solution as prepared above may be used instead), was added to each plate so that the liquid thoroughly soaked into all sheets. The sheets were positioned and then creased so that the back of the plate and one side wall were covered, two sheets were then removed and placed on a sterile surface. Along the edge of the plate across from the covered side wall 15 inoculated wheat seeds were placed evenly at least one inch from the top of the plate and half an inch from the sides. Seeds were placed smooth side up and with the pointed end of the seed pointing toward the side wall of the plate covered by germination paper. The seeds were then covered by the two reserved sheets, and the moist paper layers smoothed together to remove air bubbles and secure the seeds, and then the lid was replaced. For each treatment, at least three plates with 15 seeds per plate were prepared. The plates were then randomly distributed into stacks of 8-12 plates and a plate without seeds was placed on the top. The stacks were incubated at 60% relative humidity, and 22° C. day, 18° C. night with 12 hours light and 12 hours dark for 24 hours, then each plate was turned to a semi-vertical position with the side wall covered by paper at the bottom. The plates were incubated for an additional 5 days, then wheat seeds were scored manually for root, seedling and shoot length, root and shoot area, and seedling surface area.

Statistical analysis was performed using R (R Core Team, 2016. R: A language and environment for statistical computing. R Foundation for Statistical Computing, Vienna, Austria. R-project.org/). Improvement of plant health is shown by percent change of one or more metrics relative to controls (e.g., 10% means microbe performed 10% above control performance; −10% means microbe performed 10% below control performance). Results are shown in the following table.

TABLE 7
Plant vigor of endophyte and control treated wheat seedlings

| | | | % Delta relative |
|---|---|---|---|
| MIC-ID | Crop | Metric | to Control |
| MIC-28421 | Wheat | Root Length | 23 |
| MIC-28421 | Wheat | Shoot Length | 72 |
| MIC-28421 | Wheat | Root Length | 9 |
| MIC-28421 | Wheat | Shoot Length | 18 |

### Example 4. Method of Preparation of Endophytes and Heterologous Disposition of Endophytes on Seeds for Greenhouse Trials

Seeds were heterologously disposed to each endophyte according to the following seed treatment protocol.

### Preparation of Seeds

Sieves were used to standardize the size of seeds used for greenhouse trials. The average weight of seeds was calculated by weighing 3 samples of 100 size selected seeds each and calculating the average weight of a seed. This value was used to calculate the target dose of endophyte per seed. The target dose was generally between 10\hat{ }0-10\hat{ }6 CFU per seed, in some cases at least 10\hat{ }3 CFU per seed, or at least 10\hat{ }4 CFU per seed.

### Preparation of Fungal Endophytes

The thawed contents of a cryovial were plated on 100% MEA with 3% agar plates. The plates were sealed with Parafilm® and incubated in a growth chamber set at 60% relative humidity and 22° C. with diurnal light settings (12:12 dark to light) for approximately 14 days.

The spore suspension buffer was prepared by mixing 1 ml 10% Silwet with 250 ml 1×PBS and filter sterilizing. For each plate of fungi, 4-5 ml of the prepared sterile PBS was added, and an L-shaped spreader used to vigorously scrape the spores, tilting the plate to allow the suspension to sink to the bottom of the plate. If additional plates of the fungal endophyte were prepared, an additional 2 ml of the prepared sterile PBS and the suspension from the prior plate of the treatment were added and the scraping procedure followed as above. The suspension was then pipetted onto a piece of sterile Miracloth over a sterile collection container. Spores per ml were counted under a microscope using a hemocytometer. The total spore suspension needed to treat the seeds with the desired dose was calculated. The target dose was generally between 10\hat{ }0-10\hat{ }6 CFU per seed, in some cases at least 10\hat{ }3 CFU per seed, or at least 10\hat{ }4 CFU per seed. The spore suspension was diluted with sterile 1×PBS so that the total volume of inoculum per seed was about 2.5 μl/seed for corn, about 1.5 μl/seed for wheat and soy, and about 1.5 μl/seed for cotton. Control treatments were prepared using equivalent volumes of sterile 1×PBS. The fungal inoculum solution was applied to the prepared seeds and mixed well.

### Example 5. Greenhouse Assessment of Improved Plant Characteristics Under Water Limiting Conditions

This example describes exemplary methods to evaluate performance of novel microbes described herein, applied as a seed treatment, in promoting plant growth of endophyte-treated corn, soy, wheat, sorghum, peanuts and/or cotton plants under water limited conditions (e.g., water deficit).

Experimental Design 18 total reps were used, reps were positioned horizontally across 4 tables with reps 1 and 2 going vertically down either side of 4 tables and reps 3-18 positioned horizontally across 4 tables.

Seed Preparation Seeds were poured through large, small, or optionally, medium sized sieves. The sieves were gently shaken until medium sized seeds remained on the tray. Seeds were examined by hand, removing all damaged and abnormally small or large seeds. Next, the appropriate number of ml of total inoculum of the seed treatment was added to a centrifuge tube. The seed treatment was centrifuged and added to the seeds.

Soil and pot preparation Nu-trays with Nu-pots were filled with well mixed soil. Pot weight was adjusted to 1,000 g/pot (+ or −5 g) immediately after the pot was filled. Each pot was covered with a thick plastic cover on the top to keep the soil from drying out until the day before planting. One day before planting, pot soil was pre-wet using an irrigation boom and Dosatron to deliver 125 ml of 1×normal Hoagland's solution per pot.

Planting One day after pre-wetting the soil, a 3-prong seed punch was used to punch three holes in the center of each pot. 6 seeds were planted per pot for soy and cotton plants and 9 seeds were planted per pot for wheat, rice, and corn plants. Stakes were placed into the pots on randomization scheme and layout. Seeds were placed into a plastic beaker and covered with 300 ml of prepared soil for soy, cotton, wheat, and corn plants, or with 125 ml of prepared soil for rice plants. Finally, the soil was flattened, and 80 ml of water was added for soy, cotton, wheat, and corn plants, and 60 ml of water was added for rice plants.

Trial management, thinning, and irrigation. Each trial was checked daily for emergence and soil water status for each pot. Irrigation occurred on Monday, Wednesday, and Friday with 125 ml of regular Hoagland's nutrient solution. Additional irrigation was performed as needed (for example, if soil surface was dry). After the final day of emergence count, each pot was thinned to one plant per pot. Each plant was checked daily for abnormal plant morphology, plant color, architecture change, or damaged plants.

Introducing water limiting conditions. On day 14, soil was saturated by adding 150 ml of Hoagland's solution (8 mM N) per pot, and letting the soil soak up all solution for 1 hour. Another 150 ml Hoagland's solution was then added to each pot (total of 300 ml added) and each pot was checked and confirmed for saturation. Onward watering was then withheld until the targeted drought stress level (such as, 80% of plants reach a wilting score of 4) was achieved. Wilting was scored on a 1-9 scale with 9=best (no wilting), 5=all leaves are rolling and soft; and 1=worst (plant almost dead). The Wilting score was recorded when the first 10-20% of plants in the trial showed early signs of wilting. Plants were visually assessed and assigned a Wilting score according to the Wilting Scoring Chart (Table 8). On the second day and third after the first wilting score, the wilting score was recorded twice daily, once in the morning and once in the afternoon. On the fourth and later days, the Wilting score was taken daily until the majority of plants in the trial scored lower than 5. At that point, all pots were re-watered and the soil re-saturated by adding 150 ml of Hoagland's solution (8 mM N) per pot, letting the soil soak up all solution for 1 hour, and then adding another 150 ml Hoagland's solution (total of 300 ml added).

Data Collection Emergence was counted on Days 4, 5 and 7 post planting for soybean, wheat, cotton, rice and counted on Days 3, 4 and 5 post planting for corn. Leaf data was collected on Days 10, 17 and 24 by counting the number of fully expanded leaves on the main stem. Data was collected from 1 out of every 3 reps. Plants were harvested at 4 weeks post planting. For each pot, plants were pulled from the soil, the shoots harvested, and the shoot biomass dried. The drying process varied depending on the size of the plant. To ensure complete drying, 5-6 samples were weighed (with bag) for 3 consecutive days to ensure weight was constant (0% moisture) in the last two days. Tiller number, head number, flowering, shoot fresh weight, total grain weight, grain metrics, including nutrition composition, days to flower, harvest index, emergence, kernel row number, leaf nutrient composition, days to silk, days to shed, plant height and anthesis silk interval were also measured and recorded.

TABLE 8
Description of phenotypes for each wilt scores

| Wilt | |
|---|---|
| score | Description of wilt phenotype |
| 9 | Normal no wilting - turgid green healthy |
| 8 | Leaves start losing turgor but are not soft yet no folding or |
| | rolling or change of leaf surface some small area of leaves |
| | becomes pale between the minor veins |
| 7 | Leaves further lose turgor become soft and pale at least one |
| | leaf starts slightly rolling |
| 6 | Leaves are further soft and pale all leaves are rolling except |
| | the center growing leaf |
| 5 | All leaves are very soft and pale with rolling - one leaf may |
| | be completely closed |
| 4 | Whole plant looks very bad - center leaves are very pale and |
| | rolling badly - all leaves may be completely closed leaf |
| | sheath starts losing turgor |
| 3 | Leaf base is still fresh - leaf sheath loses turgor 2 lower |
| | leaves start drying |
| 2 | Center leaf starts drying - leaf base is not fresh anymore - |
| | all leaves are dried |
| 1 | For any plant that is worse than score 2 - the wilting score |
| | will be 1 |

TABLE 9
Water deficit assay of endophyte and control treated corn seedlings

| | | | | % Delta relative |
|---|---|---|---|---|
| MIC-ID | Crop | Stressor | Metric | to Control |
| MIC-28421 | Corn | Water Deficit | Shoot Dry | −5 |
| | | | Weight | |
| MIC-28421 | Corn | Water Deficit | Kernel Row | 6 |
| | | | Number | |
| MIC-28421 | Corn | Water Deficit | Leaf S | 30 |
| MIC-28421 | Corn | Water Deficit | Total Grain | 18 |
| | | | Weight | |
| MIC-28421 | Corn | Water Deficit | Leaf B | 5 |
| MIC-28421 | Corn | Water Deficit | Days to Silk | 0 |
| MIC-28421 | Corn | Water Deficit | Leaf Al | −2 |
| MIC-28421 | Corn | Water Deficit | Leaf Cu | 9 |
| MIC-28421 | Corn | Water Deficit | Leaf Ca | 15 |
| MIC-28421 | Corn | Water Deficit | Anthesis Silk | −2 |
| | | | Interval | |
| MIC-28421 | Corn | Water Deficit | Leaf Mg | 22 |
| MIC-28421 | Corn | Water Deficit | Leaf Zn | −1 |
| MIC-28421 | Corn | Water Deficit | Leaf Na | 68 |
| MIC-28421 | Corn | Water Deficit | Shoot Fresh | 0 |
| | | | Weight | |
| MIC-28421 | Corn | Water Deficit | Leaf K | 10 |
| MIC-28421 | Corn | Water Deficit | Plant Height | −1 |
| MIC-28421 | Corn | Water Deficit | Leaf P | 2 |
| MIC-28421 | Corn | Water Deficit | Leaf Mn | 0 |
| MIC-28421 | Corn | Water Deficit | Leaf N | 10 |
| MIC-28421 | Corn | Water Deficit | Leaf Fe | −2 |
| MIC-28421 | Corn | Water Deficit | Harvest Index | 5 |
| MIC-28421 | Corn | Water Deficit | Days to Shed | 0 |
| MIC-28421 | Corn | Water Deficit | Days to Silk | −1 |
| MIC-28421 | Corn | Water Deficit | Total Grain | 28 |
| | | | Weight | |
| MIC-28421 | Corn | Water Deficit | Leaf Na | 25 |
| MIC-28421 | Corn | Water Deficit | Leaf B | 21 |
| MIC-28421 | Corn | Water Deficit | Leaf Mg | 17 |
| MIC-28421 | Corn | Water Deficit | Leaf S | 17 |
| MIC-28421 | Corn | Water Deficit | Shoot Fresh | 15 |
| | | | Weight | |
| MIC-28421 | Corn | Water Deficit | Leaf K | 11 |
| MIC-28421 | Corn | Water Deficit | Leaf Ca | 10 |
| MIC-28421 | Corn | Water Deficit | Leaf Mn | 9 |
| MIC-28421 | Corn | Water Deficit | Harvest Index | 8 |
| MIC-28421 | Corn | Water Deficit | Leaf Cu | 5 |
| MIC-28421 | Corn | Water Deficit | Kernel Row | 4 |
| | | | Number | |
| MIC-28421 | Corn | Water Deficit | Leaf N | 1 |
| MIC-28421 | Corn | Water Deficit | Leaf P | 1 |
| MIC-28421 | Corn | Water Deficit | Plant Height | 0 |
| MIC-28421 | Corn | Water Deficit | Leaf Fe | −1 |
| MIC-28421 | Corn | Water Deficit | Leaf Zn | −6 |
| MIC-28421 | Corn | Water Deficit | Leaf Al | −17 |
| MIC-28421 | Corn | Water Deficit | Anthesis Silk | −4 |
| | | | Interval | |
| MIC-28421 | Corn | Water Deficit | Emergence | −12 |
| MIC-28421 | Corn | Water Deficit | Shoot Dry | 1 |
| | | | Weight | |

TABLE 10
Water deficit assay of endophyte and
control treated soybean seedlings

| | | | | % Delta relative |
|---|---|---|---|---|
| MIC-ID | Crop | Stressor | Metric | to Control |
| MIC-28421 | Soybeans | Water | Shoot | −1 |
| | | Deficit | Dry Weight | |

TABLE 11
Water deficit assay of endophyte and
control treated wheat seedlings

| | | | | % Delta relative |
|---|---|---|---|---|
| MIC-ID | Crop | Stressor | Metric | to Control |
| MIC-28421 | Wheat | Water | Shoot | 1 |
| | | Deficit | Dry Weight | |

### Example 6. Greenhouse Assessment of Improved Plant Characteristics Under Water Deficit Conditions

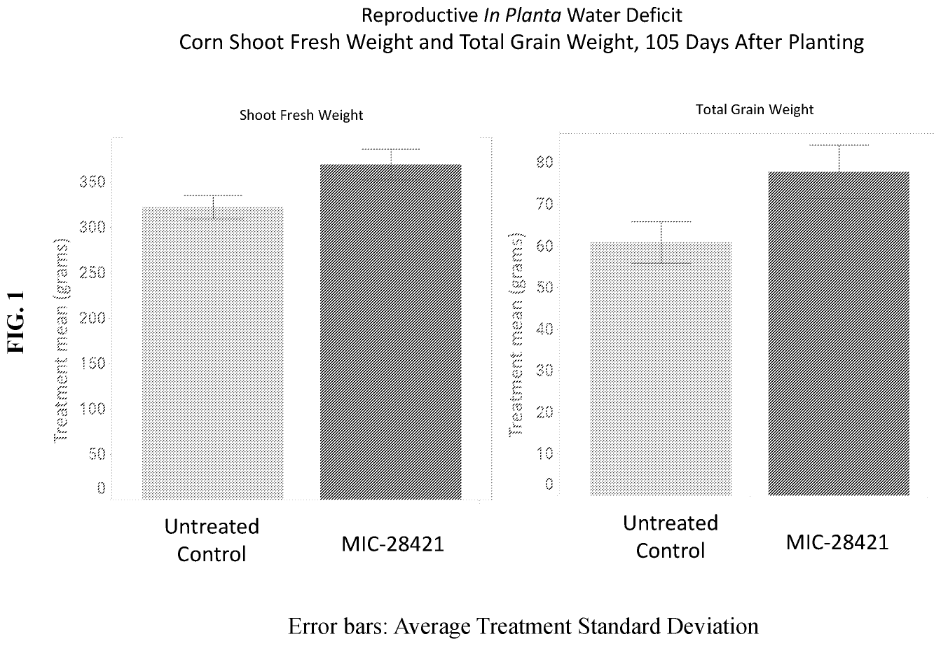

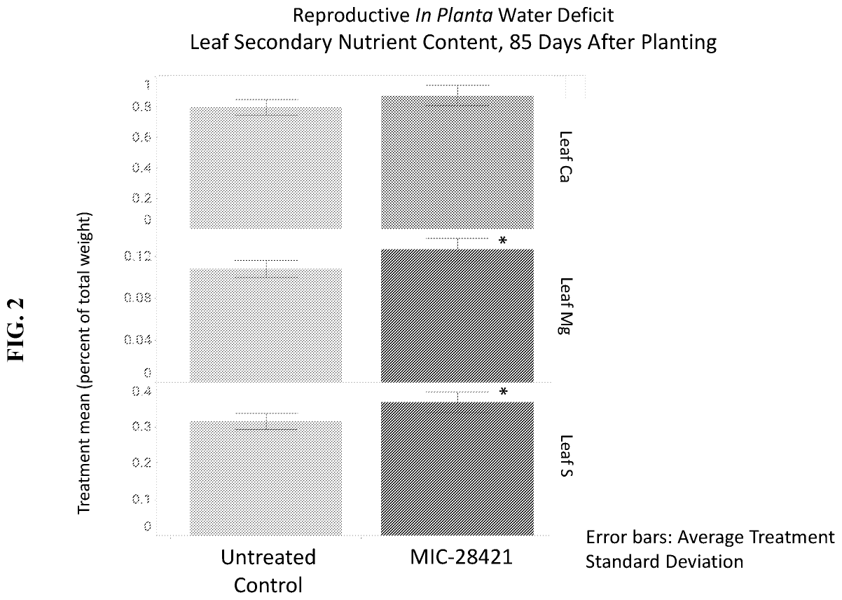

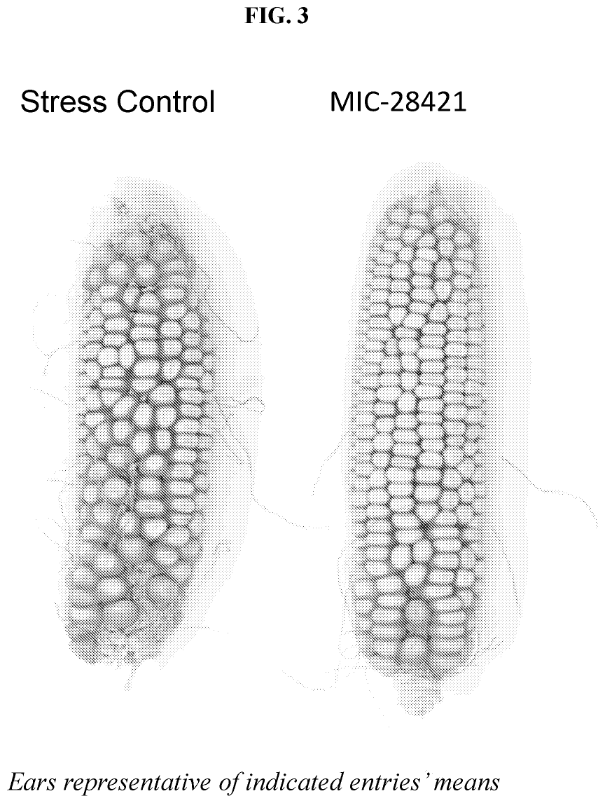

Greenhouse assay setup: This greenhouse assay was conducted in individual plastic pots, filled with moistened potting soil. This greenhouse assay was conducted using corn seeds coated with one or more of the endophytes described herein and untreated controls. Seeds were placed into each pot and lightly covered with potting mix. Replicated pots of each treatment were set up. The plants were grown in the greenhouse receiving limited water for 105 days, at which time the shoot fresh (wet) weight of the plants was collected, the grain harvested and weighed. Final growth stage of plants at harvest is approximately R2. The shoot wet weights and grain weights of MIC-28421 treated corn were significantly greater than shoots and grain of untreated corn grown in the same water deficient conditions, the results of which are shown in FIG. 1. Leaf secondary nutrient content was measured 85 days after planting; MIC-28421 treated corn showed significantly increased magnesium and sulfur content compared to untreated controls, the results of which are shown in FIG. 2. Corn ears treated with MIC-28421 showed improved grain fill compared to untreated controls. Images of representative ears indicative of the treatment mean for MIC-28421 and untreated control plants are shown in FIG. 3.

TABLE 12
Meta-analysis of MIC-28421 across greenhouse
reproductive water deficit assays in corn

| | | | Average | | |
|---|---|---|---|---|---|
| | | Days after | value | Percent | |
| Treatment | Yield metric | planting | (Grams) | delta | p-value |
| Stress Control | Total Grain | 107 | 61.16 | | — |
| | Weight | | | | |
| MIC-28421 | Total Grain | 107 | 82.2 | +34% | 0.0582* |
| | Weight | | | | |
| Stress Control | Primary Ear | 71 | 26.87 | | — |
| | Fresh Weight | | | | |
| MIC-28421 | Primary Ear | 71 | 41.91 | +56% | 0.476 |
| | Fresh Weight | | | | |
| Stress Control | Primary Ear | 71 | 44.72 | | — |
| | Fresh Weight | | | | |
| MIC-28421 | Primary Ear | 71 | 61.14 | +37% | 0.154* |
| | Fresh Weight | | | | |
| Stress Control | Yield | — | 45.49 | | — |
| MIC-28421 | Yield | — | 59.1 | +30% | 0.0348* |

TABLE 13
Summary performance of MIC-93265, MIC-93265, and
the combination of MIC-93265 & MIC-93265 stack
in the reproductive water deficit assay in corn

| | Harvest Index | | | Primary Ear Fresh Weight | | |
|---|---|---|---|---|---|---|
| | Average | Percent | p- | Average | Percent | p- |
| Treatment | (Index) | delta | value | (Grams) | delta | value |
| Untreated | 0.055 | — | — | 44.72 | — | — |
| MIC-93265 | 0.05 | −9.09091 | 0.84 | 43.86 | −1.92 | 0.9 |
| MIC-28421 | 0.073 | 32.72727 | 0.2* | 61.14 | 36.72 | 0.15* |
| MIC-28421 & | 0.064 | 16.36364 | 0.3 | 49.94 | 11.67 | 0.41 |
| MIC-93265 | | | | | | |

TABLE 14
Summary performance of MIC-93265, MIC-93265, and
the combination of MIC-93265 & MIC-93265 stack
in the reproductive water deficit assay in corn

| | Root Area | | | Shoot Fresh Weight | | |
|---|---|---|---|---|---|---|
| | Average | Percent | | Average | Percent | |
| Treatment | (cm²) | delta | p-value | (Grams) | delta | p-value |
| Untreated | 170.71 | — | — | 730.25 | — | — |
| MIC-93265 | 163.1 | −4.46 | 0.45 | 712.73 | −2.40 | 0.51 |
| MIC-28421 | 160.59 | −5.93 | 0.35 | 737.19 | 0.95 | 0.82 |
| MIC-28421 & | 158.02 | −7.43 | 0.24 | 697.44 | −4.49 | 0.2* |
| MIC-93265 | | | | | | |

Percent delta = Percent delta of each treatment versus the untreated control
p-value = p-value of the least square means difference of Student's T using the JMP Fit Least Squares program holding location (rep) and treatment as known variables
*indicates significant difference versus the Untreated control at a p-value less than or equal to 0.2

### Example 7. Method of Preparation of Endophytes and Heterologous Disposition of Endophytes on Seeds for Field Trials

### Preparation of Endophytes

An agar plug of each bacterial strain was transferred using a transfer tube to 4 ml of potato dextrose broth (PDB) in a 24 well plate and incubated at room temperature at 675 rpm on a shaker for 3 days. After growth of bacteria in broth, 200 μl was transferred into a spectrophotometer reading plate and bacteria OD was read at 600 nm absorbance. All bacteria strains were then normalized to 0.05 OD utilizing PBS 1×buffer.

### Preparation of Formulation for Seed Treatments

A 2% weight/volume solution of sodium alginate for the seed coatings was prepared by the following method. An Erlenmeyer flask was filled with the appropriate volume of deionized water and warmed to 50° C. on a heat plate with agitation using a stir bar. The appropriate mass of sodium alginate powder for the desired final concentration solution was slowly added until dissolved. The solution was autoclaved at 121° C. at 15 PSI for 30 minutes to sterilize.

Talc for the powdered seed coatings was prepared by the following method. Talc was aliquoted into bags or 50 ml Falcon tubes and autoclaved in dry cycle (121° C. at 15 PSI for 30 minutes) to sterilize.

### Heterologous Disposition of Endophytes on Seeds

Seeds treated were heterologously disposed to each endophyte according to the following seed treatment protocol.

Liquid formulation: Liquid culture was added to the seeds at a rate of 23 (for fungal endophyte treatments) or 8.4 (for bacterial endophyte treatments) ml per kg of seeds, with equivalent volumes of the prepared sodium alginate. Control treatments were prepared using equivalent volumes of sterile broth. The seeds were then agitated to disperse the solution evenly on the seeds. For fungal endophytes, 15 g per kg of seed of talc powder as prepared above was added and the seeds were agitated to disperse the powder evenly on the seeds. Then 16.6 ml (for fungal endophyte treatments) or 2.4 ml (for bacterial endophyte treatments) per kg of seed of Flo-Rite® 1706 (BASF, Ludwigshafen, Germany) was added and the seeds were agitated to disperse the powder evenly on the seeds. Slightly less Flo-Rite® was used for small grains and canola seeds, slightly more Flo-Rite® was used for seeds such as corn, soy, cotton, and peanut seeds. The target dose was generally between 10\hat{ }0-10\hat{ }6 CFU per seed, in some cases at least 10\hat{ }3 CFU per seed, or at least 10\hat{ }4 CFU per seed. Treated seeds were allowed to dry overnight in a well-ventilated space before planting.

Dry formulation: The 2% sodium alginate solution prepared above was added to the seeds at a rate of 23 ml per kg of seeds. Equal parts of dry biomass and talc prepared as above were mixed. The solution was applied so that an equivalent of 10 g of powdered dry biomass was applied per kg of seeds. Control treatments were prepared using equivalent volumes of talc. The seeds were then agitated to disperse the solution evenly on the seeds. Then 16.6 ml per kg of seed of Flo-Rite® 1706 (BASF, Ludwigshafen, Germany) was added and the seeds were agitated to disperse the powder evenly on the seeds. Slightly less Flo-Rite® was used for small grains and canola seeds, slightly more Flo-Rite® was used for seeds such as corn, soy, cotton, and peanut seeds. The target dose was generally between 10\hat{ }0-10\hat{ }6 CFU per seed, in some cases at least 10\hat{ }3 CFU per seed, or at least 10\hat{ }4 CFU per seed. Treated seeds were allowed to dry overnight in a well-ventilated space before planting.

### Example 8. Field Assessment of Improved Plant Characteristics

### Rice

Field trials were conducted, preferably, at multiple locations. In some embodiments, rice seeds were treated with commercial fungicidal and insecticidal treatment. Seeds were heterologously disposed with the endophyte treatments and formulation control (lacking the one or more heterologously disposed endophytes) as described in Example 7, untreated seeds (lacking formulation and the one or more heterologously disposed endophyte) were also planted. Seeds were sown in regularly spaced rows in soil at 1.2 million seeds/acre seeding density. At each location at least 3 replicate plots were planted for each endophyte or control treatments in a randomized complete block design. For example, each plot consisted of seven, 15.24 m (40 ft.) rows. At the end of the field trial employing endophyte treatment and control treatment plants, plots were harvested, for example, by machine with a 5-ft research combine and yield was calculated by the on-board computer.

TABLE 15
Exemplary field trial of endophyte treated rice

| | SEQ | | Field | | % Delta relative |
|---|---|---|---|---|---|
| MIC-ID | ID NO: | Crop | Trial Year | Metric | to Control |
| MIC-28421 | 29-38 | Rice | 2018 | Yield | 0.6 |
| MIC-28421 | 29-38 | Rice | 2019 | Yield | −0.9 |
| MIC-28421 | 29-38 | Rice | 2019 | Yield | 1.7 |

### Wheat

Field trials were conducted at multiple locations with multiple plots per location. Wheat seeds (optionally treated with commercial fungicidal and insecticidal treatments) were heterologously disposed with the endophyte treatments as described in Example 16, untreated seeds (lacking formulation and the one or more heterologously disposed endophyte) were also planted. Seeds were sown in regularly spaced rows in soil at 1.2 million seeds/acre seeding density. At each location at least 3 replicate plots were planted for each endophyte or control treatments in a randomized complete block design. Each plot consisted of seven, 15.24 m (40 ft.) rows.

Plots were harvested by machine, for example with a 5-ft research combine and yield and shoot dry weight were calculated by the on-board computer.

TABLE 16
Exemplary field trial of endophyte treated wheat

| | | | Field | | |
|---|---|---|---|---|---|
| | SEQ | | Trial | | % Delta relative |
| MIC-ID | ID NO: | Crop | Year | Metric | to Control |
| MIC-28421 | 29-38 | Wheat | 2017 | Yield | 3.5 |
| MIC-28421 | 29-38 | Wheat | 2018 | Shoot Dry | −2 |
| | | | | Weight | |

### Corn

Field trials were conducted at multiple locations, preferably with multiple plots per location. Plots were irrigated, non-irrigated (dryland), or maintained with suboptimal irrigation at a rate to target approximately 25% reduction in yield. In some embodiments, corn seeds were treated with commercial fungicidal and insecticidal treatment. Seeds were heterologously disposed with the endophyte treatments as described in Example 7, untreated seeds (lacking formulation and the one or more heterologously disposed endophyte) were also planted. Seeds were sown in regularly spaced rows in soil at planting densities typical for each region. At each location at least 3 replicate plots were planted per endophyte or control treatment in a randomized complete block design. For example, each plot consisted of four 15.24 m (40 ft.) rows, each separated by 76.2 cm (30 in).

At the end of the field trial employing endophyte treatment and control treatment plants, plots were harvested, for example, by machine with a 5-ft research combine and yield was calculated by the on-board computer. Only the middle two rows of the 4 row plots were harvested to prevent border effects.

TABLE 17
Exemplary field trial of endophyte treated corn

| | | Field | | % Delta relative |
|---|---|---|---|---|
| MIC-ID | Crop | Trial Year | Metric | to Control |
| MIC-28421 | Corn | 2018 | Yield | 1.8 |
| MIC-28421 | Corn | 2019 | Yield | 1.7 |
| MIC-28421 | Corn | 2019 | Yield | −1.5 |

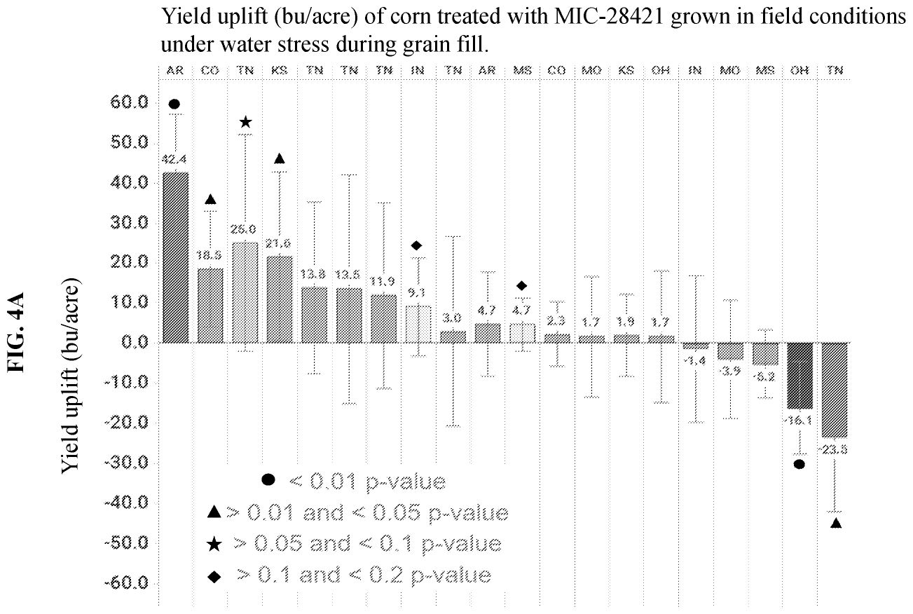

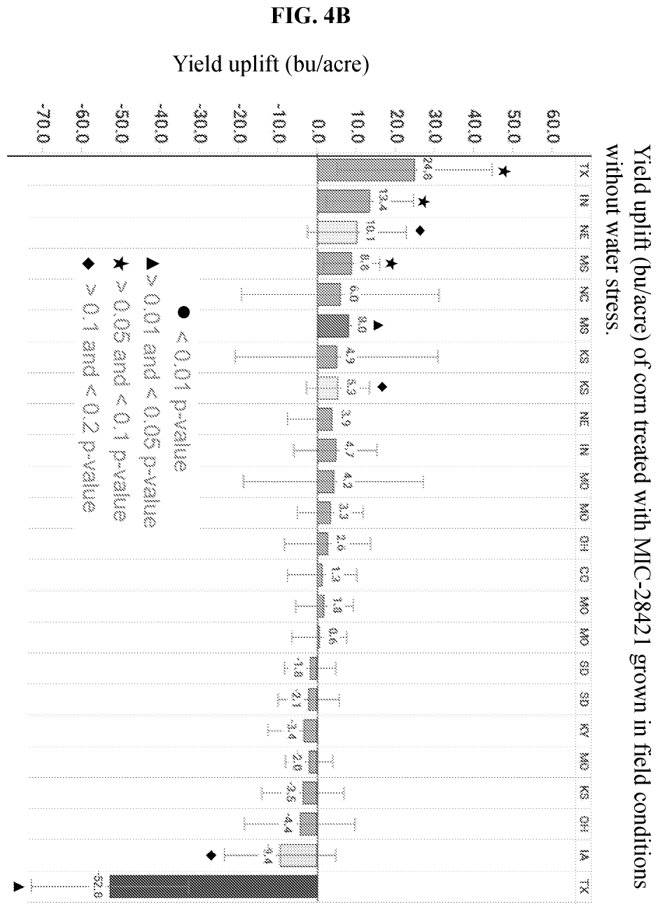

Forty-four field trials were conducted in 2019 and spring 2020 across AR, CO, IA, IN, KS, KY, MO, MS, NC, NE, OH, OK, SD, TN, and TX. In trials not under drought stress, corn plants grown from seeds treated with MIC-28421 showed an average of 1 bu/acre yield uplift, representing an average of +0.5% increase in yield, and a 67% win rate. In trials under water stress during grain fill, where water stress under grain fill represents 3.5 inches of required water or less from flowering through physiological maturity, corn plants grown from seeds treated with MIC-28421 showed an average of 6.3 bu/acre increase yield uplift, representing an average of 3.8% increase in yield, and a 75% win rate. Results are shown in FIG. 4A and FIG. 4B.

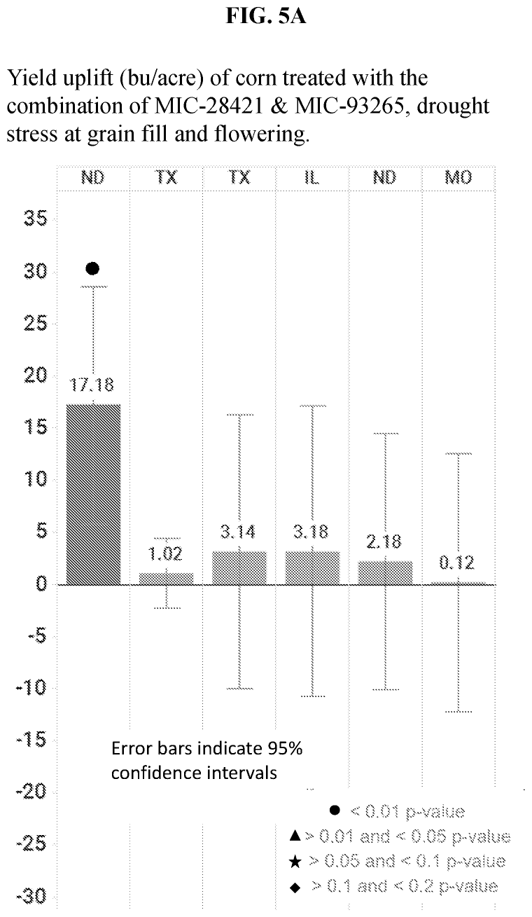

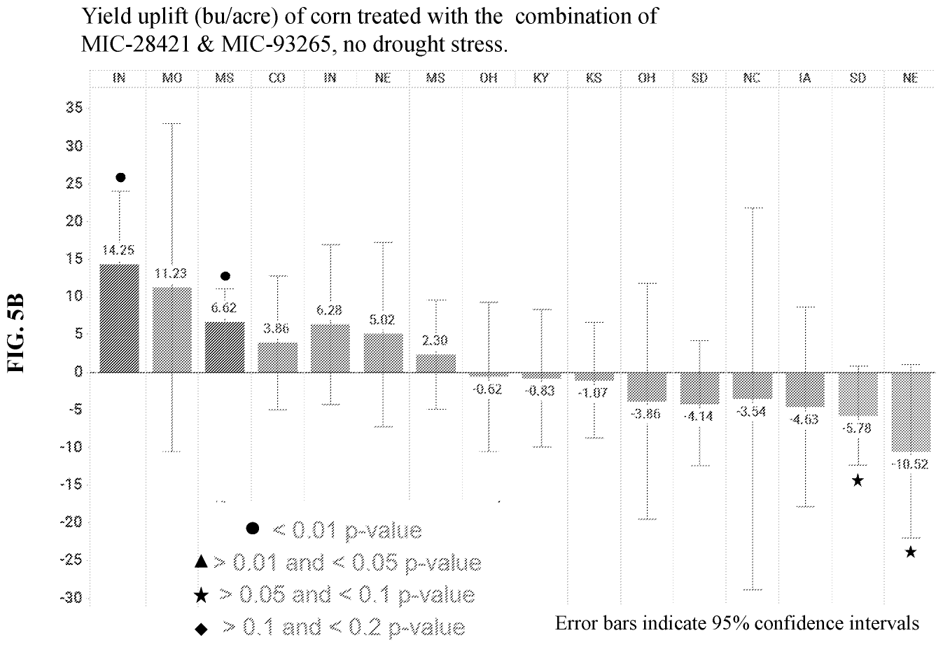

Twenty-two field trials were conducted in spring 2020 across CO, IA, IL, IN, KS, KY, MO, MS, NC, ND, NE, OH, SD, and TX. In trials not under drought stress, corn plants grown from seeds treated with both MIC-28421 and MIC-93265 showed an average of 1.7 bu/acre yield uplift, representing an average of +0.8% increase in yield, and a 44% win rate. In trials under water stress during grain fill, where water stress under grain fill represents 3.5 inches of required water or less from flowering through physiological maturity, corn plants grown from seeds treated with both MIC-28421 and MIC-93265 showed an average of 4.8 bu/acre yield uplift, representing an average of +4.9% increase in yield, and a 100% win rate. Results are shown in FIG. 5A and FIG. 5B.

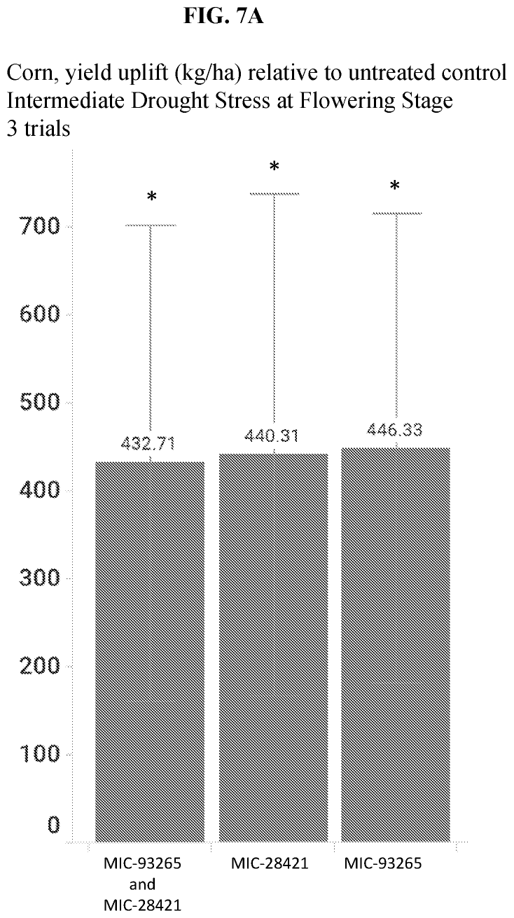

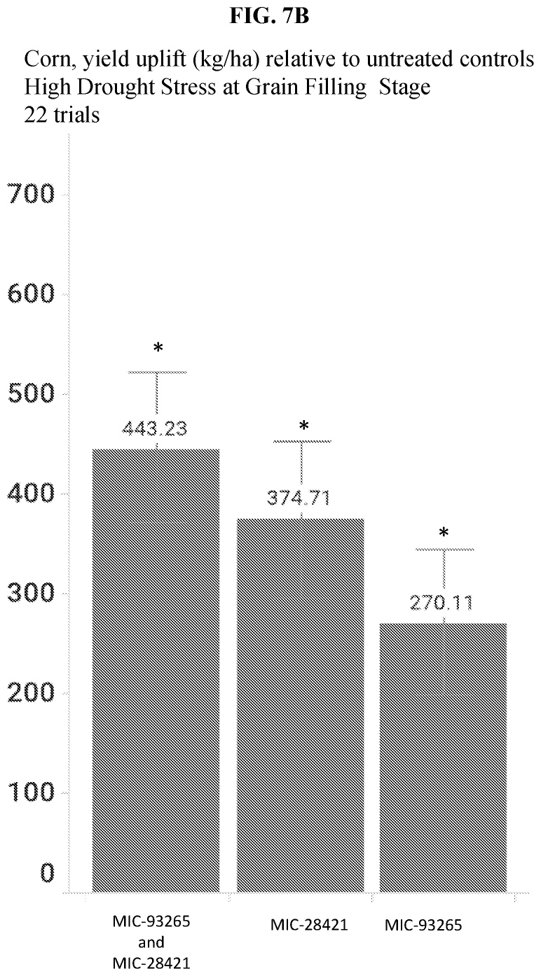

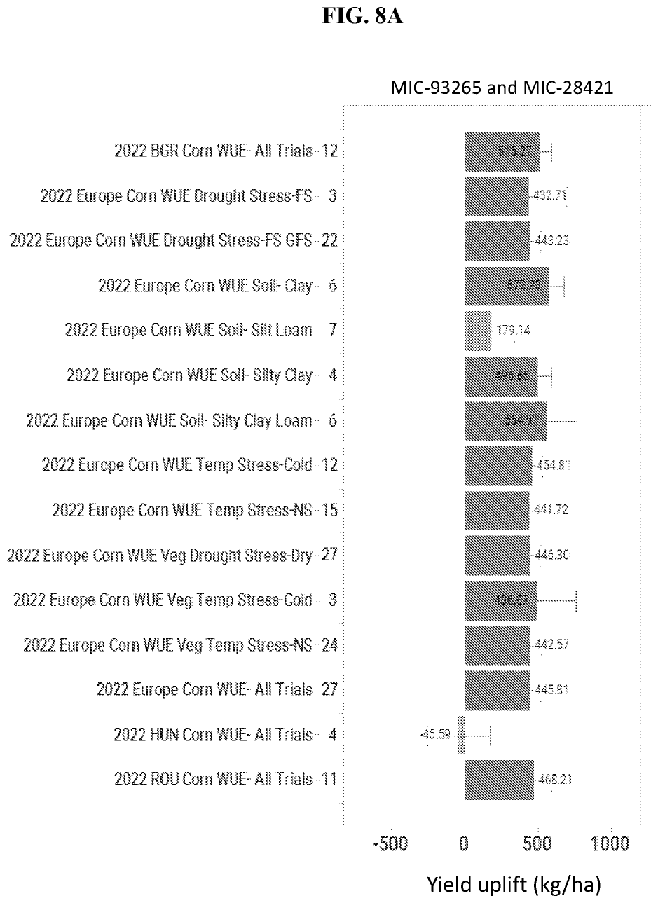

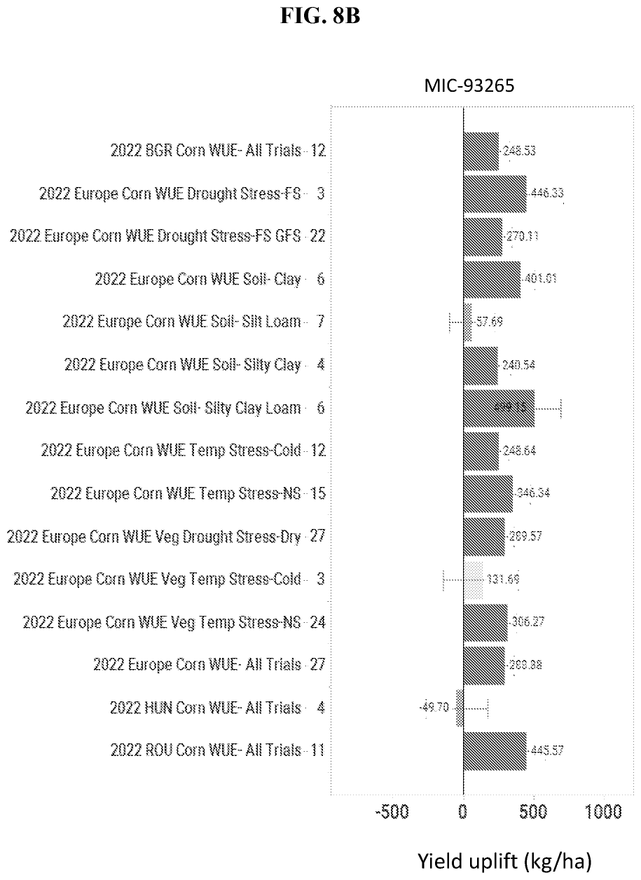

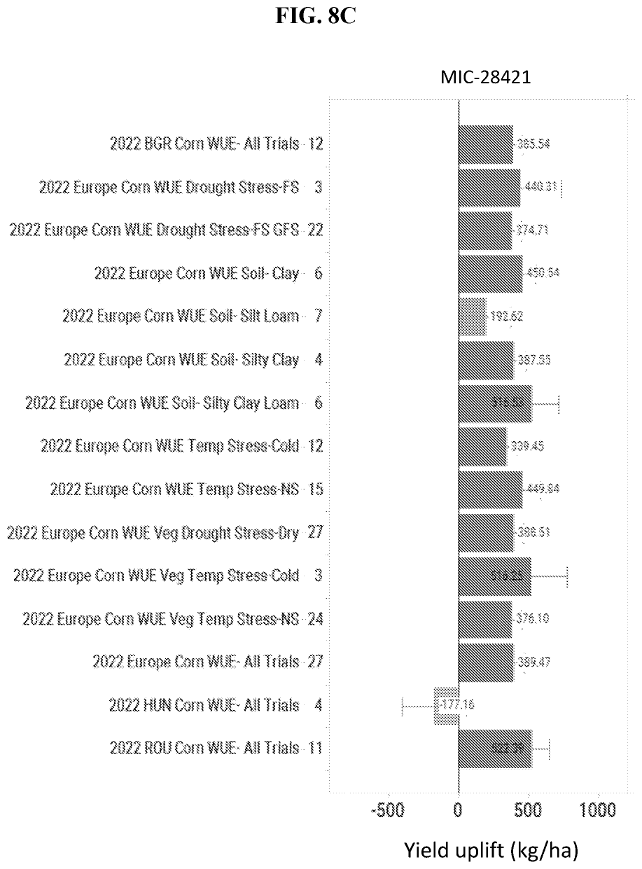

Twenty-seven field trials in corn were conducted in locations across Bulgaria, Hungary, and Romania, selected for a varieties of opportunistic stress, in particular drought. Results from three endophyte treatments were compared to untreated controls in each trial. One endophyte treatment had both MIC-28421 and MIC-93265, one endophyte treatment had only MIC-28421, and one endophyte treatment had only MIC-93265. Twenty-five of the locations showed either moderate or severe stress during flowering or grain filling. Stress during flowering is abbreviated FS in FIGS. 8A-8C. Stress during grain filling is abbreviated GFS in FIGS. 8A-8C. Eight of the twelve sites in Bulgaria received less than 200 mm of rain during the growing season; all locations in Bulgaria received less than 304 mm of rain during the growing season. Average rainfall in Bulgaria (abbreviated BG or BGR in FIGS. 8A-8C) was 194 mm. The locations in Hungary received on average of 344 mm rainfall during the growing season. Locations within Romania received an average of 295 mm rainfall during the growing season. In trials under intermediate drought stress during flowering, all three treatments showed significant improvement in yield relative to untreated controls: treatment with the combination of MIC-28421 and MIC-93265 showed an average improvement of 432.71 kg/ha relative to untreated control, treatment with MIC-28421 showed an average improvement of 440.31 kg/ha relative to untreated control, treatment with MIC-93265 showed an average improvement of 446.33 kg/ha relative to untreated control. In trials under high drought stress during flowering and grain-filling, all three treatments showed significant improvement in yield relative to untreated controls, however, treatment with the combination of MIC-28421 and MIC-93265 (443.23 kg/ha yield uplift) performed markedly better than either component individually (MIC-28421: 374.71 kg/ha, MIC-93265: 270.11 kg/ha). Results are shown in FIGS. 7A and 7B. All twenty-seven sites were characterized by soil type, rainfall and temperature. FIGS. 8A, 8B, and 8C show the three endophyte treatments performed well in clay and clay loam soils. The combination of MIC-28421 and MIC-93265 (454.81 kg/ha yield uplift) performed markedly better than either component individually (MIC-28421: 339.45 kg/ha, MIC-93265: 248.64 kg/ha).

### Soy

Field trials were conducted, preferably, at multiple locations. Seeds were heterologously disposed with the endophyte treatments as described in Example 16, untreated seeds (lacking formulation and the one or more heterologously disposed endophyte) were also planted. In some embodiments, soybean seeds were treated with commercial fungicidal and insecticidal treatment. Seeds were sown in regularly spaced rows in soil at planting densities typical for each region, for example, at 180,000 seeds/acre seeding density. At each location at least 3 replicate plots were planted per endophyte or control treatment in a randomized complete block design). For example, each plot consisted of four 15.24 m (40 ft.) rows, each separated by 76.2 cm (30 in).

At the end of the field trial employing endophyte treatment and control treatment plants, plots were harvested, for example, by machine with a 5-ft research combine and yield and shoot dry weight were calculated by the on-board computer. Only the middle two rows of the 4 row plots were harvested to prevent border effects.

TABLE 18
Exemplary field trial of endophyte treated soy

| | | Field | | % Delta relative |
|---|---|---|---|---|
| MIC-ID | Crop | Trial Year | Metric | to Control |
| MIC-28421 | Soybeans | 2018 | Yield | −2 |
| MIC-28421 | Soybeans | 2019 | Yield | −15.2 |
| MIC-28421 | Soybeans | 2019 | Yield | 2.6 |
| MIC-28421 | Soybeans | 2019 | Yield | 1.2 |
| MIC-28421 | Soybeans | 2019 | Yield | 0.2 |
| MIC-28421 | Soybeans | 2019 | Yield | 1.5 |
| MIC-28421 | Soybeans | 2018 | Shoot Dry | −4 |
| | | | Weight | |

### Cotton

Field trials were conducted, preferably, at multiple locations. Seeds were heterologously disposed with the endophyte treatments as described in Example 7, untreated seeds (lacking formulation and the one or more heterologously disposed endophyte) were also planted. In some embodiments, cotton seeds were treated with commercial fungicidal and insecticidal treatment. Seeds were sown in regularly spaced rows in soil at planting densities typical for each region, for example, at 40,000 seeds/acre seeding density. At each location at least 3 replicate plots were planted per endophyte or control treatment in a randomized complete block design). For example, each plot consisted of four 15.24 m (40 ft.) rows, each separated by 76.2 cm (30 in).

At the end of the field trial employing endophyte treatment and control treatment plants, plots were machine harvested, and yield was calculated by the on-board computer.

TABLE 19
Exemplary field trial of endophyte treated cotton

| | SEQ | | Field | | % Delta relative |
|---|---|---|---|---|---|
| MIC-ID | ID NO: | Crop | Trial Year | Metric | to Control |
| MIC-28421 | 29-38 | Cotton | 2018 | Yield | −3.2 |

### Canola

Field trials are conducted at multiple locations, preferably in diverse geographic regions. Plots are irrigated, non-irrigated (dryland) or maintained with suboptimal irrigation at a rate to target approximately 25% reduction in yield. In some embodiments, canola seeds are treated with commercial fungicidal and insecticidal treatment. Seeds are heterologously disposed with the endophyte treatments as described in Example 7, untreated seeds (lacking formulation and the one or more heterologously disposed endophyte) are also planted. At each location, at least 3 replicate plots are planted for each endophyte or control treatment in a randomized complete block design.

At the end of the field trial employing endophyte treatment and control treatment plants, plots are harvested, for example, by machine with a 5-ft research combine and yield and shoot dry weight are calculated by the on-board computer.

### Peanut

Field trials are conducted at multiple locations, preferably in diverse geographic regions. Optionally, plots are non-irrigated (dryland) or maintained with suboptimal irrigation at a rate to target approximately 25% reduction in yield. In some embodiments, peanut seeds are treated with commercial fungicidal and insecticidal treatment. Seeds are heterologously disposed with the endophyte treatments as described in Example 16, untreated seeds (lacking formulation and the one or more heterologously disposed endophyte) are also planted.

At the end of the field trial employing endophyte treatment and control treatment plants, plots are harvested, for example, by machine with a 5-ft research combine and yield and shoot dry weight are calculated by the on-board computer.

### Sorghum

Field trials are conducted at multiple locations with multiple plots per location. Sorghum seeds (optionally treated with commercial fungicidal and insecticidal treatments) are heterologously disposed with the endophyte treatments as described in Example 16, untreated seeds (lacking formulation and the one or more heterologously disposed endophyte) are also planted. Seeds are sown in randomized split plot, at a rate typical for the growing region. When irrigation is provided, crops are maintained with suboptimal irrigation to a rate to target an approximately 25% reduction in yield. Emergence counts are recorded at emergence of the first plants (e.g., approximately 4 days after planting) and again 7-10 days after the first emergence count. Emergence data is taken from the middle two rows of plants. Seedling vigor is scored on the date of the last emergence count by surveying the entire plot and rating the apparent health and extent of growth of the plants in the plot (on a scale of 1-10). After harvesting the middle two rows of plants, the test weight (lb/bushel), moisture (%), one thousand (1000) kernel weight, and yield (lb/plot and/or bu/A) is recorded. Additional metrics taken include vigor, NDVI measurements, silking, lodging score, gap count, and stand count. In some embodiments, NDVI measurements are taken at growth stages V10, R1, R3 and R5. In some embodiments, NDVI is measured at 36 inches above the crop canopy. One pass per row is made over each of the two interior rows. In some embodiments, silking date is recorded when 50% of the plants within the plot have started to silk.

### Example 9. Method of Determining Seed Nutritional Quality Trait Component: Fat

Seed samples from harvested plants are obtained as described in Example 8. Analysis of fat is conducted on replicate samples according to the Association of Official Agricultural Chemists Reference Method AOAC 920.39, of the Official Methods of Analysis of AOAC International, 20th Edition (2016), herein incorporated by reference in its entirety. Samples are weighed onto filter paper, dried, and extracted in hot hexane for 4 hrs. using a Soxlhet system. Oil is recovered in pre-weighed glassware, and % fat is measured gravimetrically. Mean percent changes between the treatment (endophyte-treated seed) and control (seed treated with the formulation but no endophyte) are calculated.

### Example 10. Method of Determining Seed Nutritional Quality Trait Component: Ash

Seed samples from harvested plants are obtained as described in Example 8. Analysis of ash is conducted on replicate samples according to the Association of Official Agricultural Chemists Reference Method AOAC 920.39, of the Official Methods of Analysis of AOAC International, 20th Edition (2016). Samples are weighed into pre-weighed crucibles, and ashed in a furnace at 600° C. for 3 hr. Weight loss on ashing is calculated as % ash. Mean percent changes between the treatment (one or more heterologously disposed endophytes) and control (lacking the one or more heterologously disposed endophytes) with the formulation but no endophytes are calculated.

### Example 11. Method of Determining Seed Nutritional Quality Trait Component: Fiber

Seed samples from harvested plants are obtained as described in Example 8. Analysis of fiber is conducted on replicate samples according to the Association of Official Agricultural Chemists Reference Method AOAC 920.39, of the Official Methods of Analysis of AOAC International, 20th Edition (2016). Samples are weighed into filter paper, defatted and dried, and hydrolyzed first in acid, then in alkali solution. The recovered portion is dried, weighed, ashed at 600°, and weighed again. The loss on ashing is calculated as % Fiber. Mean percent changes between the treatment (one or more heterologously disposed endophytes) and control (lacking the one or more heterologously disposed endophytes) with the formulation but no endophyte are calculated.

### Example 12. Method of Determining Seed Nutritional Quality Trait Component: Moisture

Seed samples from harvested plants are obtained as described in Example 8. Analysis of moisture is conducted on replicate samples according to the Association of Official Agricultural Chemists Reference Method AOAC 920.39, of the Official Methods of Analysis of AOAC International, 20th Edition (2016). Samples are weighed into pre-weighed aluminum dishes and dried at 135° C. for 2 hrs. Weight loss on drying is calculated as % Moisture. Mean percent changes between the treatment (one or more heterologously disposed endophytes) and control (lacking the one or more heterologously disposed endophytes) with the formulation but no endophyte are calculated.

### Example 13. Method of Determining Seed Nutritional Quality Trait Component: Protein

Seed samples from harvested plants are obtained as described in Example 8. Analysis of protein is conducted on replicate samples according to the Association of Official Agricultural Chemists Reference Method AOAC 920.39, of the Official Methods of Analysis of AOAC International, 20th Edition (2016). Samples are combusted and nitrogen gas is measured using a combustion nitrogen analyzer (Dumas). Nitrogen is multiplied by 6.25 to calculate % protein. Mean percent changes between the treatment (one or more heterologously disposed endophytes) and control (lacking the one or more heterologously disposed endophytes) with the formulation but no endophyte) are calculated.

### Example 14. Method of Determining Seed Nutritional Quality Trait Component: Carbohydrate

Seed samples from harvested plants are obtained as described in Example 8. Analysis of carbohydrate is determined for replicate samples as a calculation according to the following formula:

$$Total Carbohydrate = 100 \% - \% ( Protein + Ash + Fat + Moisture + Fiber )$$

Where % Protein is determined according to the method of Example 13, % Ash is determined according to the method of Example 10, % Fat is determined according to the method of Example 9, % Moisture is determined according to the method of Example 12, and % Fiber is determined according to the method of Example 11. Mean percent changes between the treatment (endophyte-treated seed) and control (seed treated with the formulation but no endophyte) are calculated.

### Example 15. Method of Determining Seed Nutritional Quality Trait Component: Calories

Seed samples from harvested plants are obtained as described in Example 8. Analysis of Calories is determined for replicate samples as a calculation according to the following formula:

Total Calories=(Calories from protein)+(Calories from carbohydrate)+(Calories from fat), where Calories from protein are calculated as 4 Calories per gram of protein (as determined according to the method of Example 13), Calories from carbohydrate are calculated as 4 Calories per gram of carbohydrate (as determined according to the method of Example 14), and Calories from fat are calculated as 9 Calories per gram of fat (as determined according to the method of Example 9). Mean percent changes between the treatment (one or more heterologously disposed endophytes) and control (lacking the one or more heterologously disposed endophytes) are calculated.

### Example 16. Additional Methods for Creating Synthetic Compositions

### Osmopriming and Hydropriming

One or more endophytes are manually or mechanically inoculated onto seeds during the osmopriming (soaking in polyethylene glycol solution to create a range of osmotic potentials) and/or hydropriming (soaking in de-chlorinated water) process. Osmoprimed seeds are soaked in a polyethylene glycol solution containing one or more endophytes for one to eight days and then air dried for one to two days. Hydroprimed seeds are soaked in water for one to eight days containing one or more endophytes and maintained under constant aeration to maintain a suitable dissolved oxygen content of the suspension until removal and air drying for one to two days. Talc and or flowability polymer are added during the drying process.

### Foliar Application

One or more endophytes are manually or mechanically inoculated onto aboveground plant tissue (leaves and stems) as a liquid suspension in dechlorinated water containing adjuvants, sticker-spreaders, and UV protectants. The suspension is sprayed onto crops with a boom or other appropriate sprayer.

### Soil Inoculation

One or more endophytes are manually or mechanically inoculated onto soils in the form of a liquid suspension, either; pre-planting as a soil drench, during planting as an in-furrow application, or during crop growth as a sidedress (e.g., application between the rows of growing crops). In some embodiments, one or more endophytes are mixed directly into a fertigation system via drip tape, center pivot or other appropriate irrigation system.

### On-Planter Inoculation

One or more endophytes are manually or mechanically inoculated on to seed on-planter in the form of a dry or liquid suspension. In some embodiments, one or more endophytes are inoculated while a planter is being loaded with seed or as the planter is actively planting. In some embodiments, one or more endophytes are inoculated onto seed via a seed tender attachment while the planter is being loaded with seed, for example by a seed treater attachment for the seed tender. In some embodiments, one or more endophytes are inoculated onto seed via a seed hopper (e.g., one or more endophytes are manually added to a seed hopper on the planter). In some embodiments, a low dust powder formulation for the one or more endophytes is used. In some embodiments, one or more endophytes are inoculated onto seed while planting, e.g., on the planter in the field before, or concurrently as the seed is being dropped into the field for planting, e.g., one or more endophytes are inoculated onto seed between the seed hopper and being dropped.

### Hydroponic and Aeroponic Inoculation

One or more endophytes are manually or mechanically inoculated into a hydroponic or aeroponic system either as a powder or liquid suspension applied directly to the rockwool substrate or applied to the circulating or sprayed nutrient solution.

### Vector-Mediated Inoculation

One or more endophytes are introduced in power form in a mixture containing talc or other bulking agent to the entrance of a beehive (in the case of bee-mediation) or near the nest of another pollinator (in the case of other insects or birds). The pollinators pick up the powder when exiting the hive and deposit the inoculum directly onto the crop's flowers during the pollination process.

### Root Wash

The method includes manually or mechanically contacting the exterior surface of a plant's roots with a liquid inoculant formulation containing one or more endophytes. The plant's roots are briefly passed through standing liquid microbial formulation or liquid formulation is liberally sprayed over the roots, resulting in both physical removal of soil and microbial debris from the plant roots, as well as inoculation with microbes in the formulation.

### Seedling Soak

The method includes manually or mechanically contacting the exterior surfaces of a seedling with a liquid inoculant formulation containing one or more endophytes. The entire seedling is immersed in standing liquid microbial formulation for at least 30 seconds, resulting in both physical removal of soil and microbial debris from the plant roots, as well as inoculation of all plant surfaces with microbes in the formulation. Alternatively, the seedling can be germinated from seed in, or transplanted into, media soaked with the microbe(s) of interest and then allowed to grow in the media, resulting in soaking of the plantlet in microbial formulation for much greater time, for example: hours, days, or weeks. Endophytic microbes likely need time to colonize and enter the plant, as they explore the plant surface for cracks or wounds to enter, so the longer the soak, the more likely the microbes will successfully be installed in the plant.

### Wound Inoculation

The method includes manually or mechanically contacting the wounded surface of a plant with a liquid or solid inoculant formulation containing one or more endophytes. Plant surfaces are designed to block entry of microbes into the endosphere, since pathogens attempt to infect plants in this way. One way to introduce beneficial endophytic microbes into plant endospheres is to provide a passage to the plant interior by wounding. This wound can take many forms, including pruned roots, pruned branches, puncture wounds in the stem breaching the bark and cortex, puncture wounds in the tap root, puncture wounds in leaves, and puncture wounds in the seed allowing entry past the seed coat. Wounds can be made using tools for physical penetration of plant tissue such as needles. Microwounds may also be introduced by sonication. The microbial inoculant can then be contacted to the wound (for example, as a liquid powder, or in a pressurized reservoir), allowing the microbes to enter and colonization the endosphere. Alternatively, the entire wounded plant can be soaked or washed in the microbial inoculant for at least 30 seconds, giving more microbes a chance to enter the wound, as well as inoculating other plant surfaces with microbes in the formulation—for example pruning seedling roots and soaking them in inoculant before transplanting is a very effective way to introduce endophytes into the plant.

### Injection

The method includes manually or mechanically injecting microbes into a plant to successfully install them in the endosphere. Plant surfaces are designed to block entry of microbes into the endosphere, since pathogens attempt to infect plants in this way. To introduce beneficial endophytic microbes to endospheres, we need a way to access the interior of the plant which we can do by puncturing the plant surface with a needle and injecting microbes into the inside of the plant. Different parts of the plant can be inoculated this way including the main stem or trunk, branches, tap roots, seminal roots, buttress roots, and even leaves. The injection can be made with a hypodermic needle, a drilled hole injector, or a specialized injection system, and through the puncture wound, can then contact the microbial inoculant as liquid, as powder, inside gelatin capsules, in a pressurized capsule injection system, or in a pressurized reservoir and tubing injection system, allowing entry and colonization by microbes into the endosphere.

### Example 17. Identification of Sequence Variants Across Core Genes

Phylogenomic analysis of whole genome sequences of endophytes can be used to identify distinguishing sequence variants. Sets of genes suitable for phylogenomic analysis as well as methods for identifying the same are well known in the art, for example Floutas et al. (2012) The Paleozoic origin of enzymatic lignin decomposition reconstructed from 31 fungal genomes. Science, 336(6089):1715-9. doi: 10.1126/science.1221748 and James T Y, Pelin A, Bonen L, Ahrendt S, Sain D, Corradi N, Stajich J E. Shared signatures of parasitism and phylogenomics unite Cryptomycota and microsporidia. Curr Biol. 2013; 23(16):1548-53. doi: 10.1016/j.cub.2013.06.057. Orthologous genes to the reference set are identified in protein data bases derived from the genome of each species. Orthologous genes can be identified in the genomes using methods well known including reciprocal best hits (Ward N, Moreno-Hagelsieb G. Quickly Finding Orthologs as Reciprocal Best Hits with BLAT, LAST, and UBLAST: How Much Do We Miss? de Crécy-Lagard V, ed. PLoS ONE. 2014; 9(7):e101850. doi:10.1371/journal.pone.0101850) and Hidden Markov Models (HMMs). The best hits are extracted and a multiple sequence alignment generated for each set of orthologous genes. The alignments are used to build phylogenetic trees using methods well known in the art including Bayesian inference and maximum likelihood methods, for example using software tools MrBayes (Huelsenbeck, J. P. & Ronquist (2001) MRBAYES: Bayesian inference of phylogenetic trees. Bioinformatics, 17(8):754-755) and RAxML (Stamatakis, A. (2014) RAxML version 8: a tool for phylogenetic analysis and post-analysis of large phylogenies. Bioinformatics, 30 (9): 1312-1313. doi: 10.1093/bioinformatics/btu033). Sequence variants which distinguish between closely related species are identified.

### Example 18. Identification of Unique Genes in an Endophyte of Interest

Whole genome analysis of endophytes was used to identify genes whose presence, absence or over or under representation (“differential abundance”) are associated with desirable phenotypes. Key genomic features were identified by sequencing the genome of MIC-28421 searching for the presence of features using standard sequence alignment tools such as BLAST and bowtie (Langmead B, Trapnell C, Pop M, Salzberg SL. Ultrafast and memory-efficient alignment of short DNA sequences to the human genome. Genome Biol 10:R25). MIC-28421 has all the gene encoding enzymes for nitrate assimilation. It has an ammonia transporter for ammonia uptake and urease and its accessory proteins for mobilization of urea. MIC-28421 genome contains multiple phosphatase gene sequences indicative of its capability to mineralize soil organic phosphates, and also contains siderophore biosynthetic gene clusters and siderophore transporter genes. Siderophores mobilize soil iron for plant uptake.

To identify genes with differential abundance in the genome of an endophyte of interest, protein sequences predicted from the genomes of the endophyte and closely related species are compared in an all-vs-all pairwise comparison (for example, using BLAST) followed by clustering of the protein sequences based on alignment scores (for example, using MCL: Enright A. J., Van Dongen S., Ouzounis C. A. An efficient algorithm for large-scale detection of protein families. Nucleic Acids Research 30(7):1575-1584 (2002)). Additional software tools useful for this analysis are well known in the art and include OMA, OrthoMCL and TribeMCL (Roth A C, Gonnet G H, Dessimoz C. Algorithm of OMA for large-scale orthology inference. BMC Bioinformatics. 2008; 9:518. doi: 10.1186/1471-2105-9-518, Enright A J, Kunin V, Ouzounis C A. Protein families and TRIBES in genome sequence space. Nucleic Acids Res. 2003; 31(15):4632-8.; Chen F, Mackey A J, Vermunt J K, Roos D S. Assessing performance of orthology detection strategies applied to eukaryotic genomes. PLoS One. 2007; 2(4):e383.). The protein clusters are queried to identify clusters with differential abundance of proteins derived from endophytes having desirable phenotypes. Proteins of these clusters define the unique properties of these endophytes, and the abundance of genes encoding these proteins may be used to identify endophytes of the present invention.

### Example 19. Determining the Capability of Endophytes to Utilize ACC as a Sole Nitrogen Source

The methods of the following example enable the detection of bacteria which possess the ability to utilize 1-aminocyclopropane-1-carboxylate (ACC) as a sole nitrogen source. This method was adapted from the principles in Patil et al. 2016 “Improved method for effective screening of ACC(1-aminocyclopropane-1-carboxylate) deaminase-producing microorganisms”. In the present example, bacteria are tested in a high throughput format along with positive and negative controls. Bacteria capable of utilizing ACC as a sole nitrogen source produce ammonia in the process of catabolizing ACC. This ammonia causes a pH change and a change of the phenol red indicator dye from yellow/orange to pink.

### ACC Agar Solution Preparation

Trace element solution. 70 mL deionized water, 10 mg Boric acid, 11.19 mg Manganese sulfate monohydrate, 124.6 mg Zinc sulfate heptahydrate, 78.22 mg Cupic sulfate pentahydrate, and 10 mg Sodium molybdenum oxide dihydrate are combined then brought to a volume of 100 mL.

Iron solution. 100 mg of Iron sulfate heptahydrate and 10 mL of deionized water are combined, then the solution is filter sterilized.

Preparation of ACC media. 4.0 g KH2PO4, 6.0 g Na2HPO4, 0.2 g MgSO4 7H2O, 2.0 g glucose, 2.0 g D-gluconic acid, 0.1 ml of the trace elements solution, 0.1 ml of the iron solution, and 1000 ul 0.5% Phenol red are combined. The ACC media is brought to a pH of 7.2 and brought to a volume of 1 L. 18 g of bacto-agar is then added to the prepared media and autoclaved. 100 ul of the media is added to each well of a 96-well plate.

Application of ACC media. ACC media is prepared in water at 25 mg/ml. The media is filter sterilized. 6 μl of the sterilized media is added into each well of 3 plates of DF media. 6 μl of sterile water is added to 3 “No-ACC” control plates.

Preparation of bacteria. The bacteria of interest is inoculated in 24 well plates with 3 mL of TSB and grown for three days at room temperature. A positive control (Burkholderia strain having known ability to use ACC as a sole nitrogen source) and negative control (E. coli) are grown in the same conditions. On the third day of growth, the bacteria are normalized to an initial OD₆₀₀ of 0.2 in 1×phosphate buffered saline buffer.

Stamping the bacteria onto Pikovskaya agar. 2 μl of each bacterial culture is stamped into media containing ACC in triplicate and 3 “No-ACC” control plates. All four plates are then placed in 24° C. chamber for growth for 7 days.

Imaging and Analyzing. Each plate is photographed. Color change is scored by eye. A color change in the ACC containing plates relative to the plates not containing ACC is considered a positive result for that microbe. Microbes indicating positive results for more than 50% of replicates tested are considered to be capable of utilizing ACC as their sole nitrogen source.

### Example 20. Determining the IAA Production Capability of Endophytes

The methods of the following example enable the identification of microbes able to produce the phytohormone indole-3-acetic acid (IAA). In the present example, microbes are tested in a high throughput panel, by inoculation into an amended media, incubation, and color change reaction. Colorimetric estimations of the oxidation of indole-3-acetic acid (IAA) are based on the Salkowski reaction with unoxidized IAA in the oxidation reaction mixture. This reagent yields a pink color with IAA. The intensity of the color diminishes in proportion to the IAA oxidized.

IAA media and reagent preparation. The microbes are inoculated into Tryptic Soy broth amended with 0.1% 1-tryptophan (“TSB with 1-trp”) in replicates of three. This media is available from Sigma. The TSB media with media contains 30 g Tryptic Soy Agar, 1 g L-Trytophan, and 1 L dH2O. A modified Salkowski's Reagent (ERIC GLICKMANN AND YVES DESSAUX, A Critical Examination of the Specificity of the Salkowski Reagent for Indolic Compounds Produced by Phytopathogenic Bacteria) is used to produce the color change with the microbe's supernatant and is made day of. The Salkowski's Reagent contains 2 ml 0.5 mM FeCl₃·6H₂O, 63 ml dH2O, and 37 ml sulfuric acid.

Experimental Methods. Microbes are prepared and inoculated at a normalized concentration of 0.2 OD into three replicate 96-well plates. The plates are filled with 600 ml of Tryptic Soy broth amended with 0.1% 1-tryptophan (“TSB with 1-try”). Each plate includes a positive control, a Pantoea ananatis strain known to produce IAA, and a negative control, E. coli 5H a. They are grown for three days under agitation at a temperature of 24° C., and with gas exchange in a controlled environment. The plates are spun down in a centrifuge at 5000 rpm for four minutes. After spinning, 100 ul of the supernatant is extracted off. That supernatant is placed into a 96-well costar plate and then 100 μl of Salkowski's reagent is added. The plates are then placed in a dark space for 30 minutes and allowed to incubate for full color change.

Data Collection. One method for data collection involves image capture under standard, controlled light conditions exactly 30 minutes after adding reagents. Experimental 96 well plates are captured in both jpeg and CR2 formats. Images are then referenced for data capture. Any color change away from the negative control is recorded as a positive result, meaning IAA is produced by the given microbe. Microbes indicating positive results for more than 50% of replicates tested are considered to have IAA production capability.

### Example 21. Determining the Nitrogen Utilizing Capability of Endophyte Strains

The methods of the following example are an indication of bacteria able to utilize N from a number of sources including the atmosphere, limited amounts from residual N rich medias, or possibly from within its own cellular mass. In the present example, bacteria are normalized to 0.2 OD and 2.0 μl of each sample are stamped onto individual wells of a 96-well costar plate containing 100 μl of Nitrogen free media containing the colorimetric dye bromothymol blue. The bacteria are grown for three days at 24° C. Bacteria capable of fixing atmospheric nitrogen, or utilizing it from residual growth medium, or from its own cellular mass will begin to grow, the growth of which induces the production of ammonium. This basic compound causes the pH change within the environment to shift, driving the color of the media to turn from its original green color, to blue.

Nitrogen Free agar media preparation. In 1 L of Di H2O the following is added: 5.0 g Malic Acid, 0.5 g K₂HPO₄, 0.2 g MgSO₄·7H₂O, 0.01 g NaCl, 0.02 g CaCl₂, 2 ml bromthymol blue solution: 0.5% (w/v) in 0.2N KOH, 4 ml FeEDTA solution:1.64% (w/v) in nH₂O, 1 ml CuSO₄·2H₂O solution: 0.124% (w/v) in nH₂O, 100 μl ZnSO₄·7H₂O solution: 0.24% (w/v) in nH₂O, 1 ml H₃BO₃ solution: 0.28% (w/v) in nH₂0, 1 ml Na₂MoO₄·2H₂O solution: 0.2% (w/v) in nH₂O, 1 ml MnSO₄—H₂O solution:0.3% (w/v) in nH₂O, and 15 g agar. The final pH is adjusted to 6.8 and the solution is then autoclaved on L2 cycle for sterilization. Then, 100 μl of media is poured into each well of a 96 well costar plate.

Preparation of bacteria. The bacteria of interest are inoculated in 24 well plates with 3 mL of TSB and grown for three days at room temperature. On the third day, the growth of the bacteria is normalized by reading a 200 μl suspension of half bacteria TSB growth and half PBS 1× buffer at an absorbance of 600 nm and normalized to 0.02 to standardize the growth of the bacteria. One well is reserved for the positive control (a Pantoea ananatis strain known for its nitrogen utilization capabilities) and two wells are reserved for the negative controls (E. coli DH5a and B. thuringiensis).

Stamping the bacteria onto Pikovskaya agar. From the master plate a 1:10 serial dilution of the 96 bacteria is created. Two replicates of two dilutions (undiluted and 1:10) are created by stamping 2 μl of each bacteria onto the omni tray. Plates are then placed in 24° C. chamber for growth.

Imaging and Analyzing. Each plate is photographed. A jpeg image is taken and stored for analysis to be performed. The media color is scored by eye. Microbes indicating positive results for more than 50% of replicates tested are considered to have nitrogen utilizing capability.

### Example 22. Determining Nitrogen Fixing Capability of Endophyte Strains

Preparation of media. 39.10 g of Jensen's broth is mixed with 15.0 g agarose per 1,000 ml of Di H20. A stirbar is added to the mixture and autoclaved twice. 60 ml of media is added to an omnitray. The media is then allowed to solidify.

Culture Preparation. A sterile loop is used to inoculate bacteria into each well of a 24-well plate. One well is reserved for the positive control (a Pantoea ananatis strain known for its nitrogen fixing capabilities), two wells are reserved for the negative controls (E. coli DH5a and B. thuringiensis), and one well is left for un-inoculated media. The plate is then covered with a breathable membrane and incubated at room temperature in a 400 rpm shaker for 3 days.

Bacterial Normalization. The cultures are then centrifuged, supernatant removed and re-suspended in 1×PBS solution, and diluted 1:2 for OD reading. The concentration of each bacteria is adjusted by diluting in PBS to 0.05 OD. 1:10 dilutions in 1×PBS are then created in a 96 well plate.

Media Inoculation, Incubation and colony scoring. 2 μl of each bacteria onto TSA media omni trays. The inoculated omni trays are parafilmed and inverted to prevent condensation from forming on the lid and are stored at room temperature for 72 hours. Colony growth greater than the negative control indicates a positive result. Microbes indicating positive results for more than 50% of replicates tested are considered to have nitrogen fixing capability.

### Example 23. Determining the Phosphorous Solubilizing Capability of Endophyte Strains

The methods of the following example enable the detection of phosphate solubilizing bacteria, where the bacteria in question solubilizes tri-calcium phosphate (a form biologically unavailable to plants) into phosphate. In the present example, bacteria are tested in sets along with positive and negative controls. Bacteria capable of solubilizing the tri-calcium phosphate are identified by the formation of a “halo” around the site of growth.

Pikovskaya agar solution preparation. 31.3 grams of Pikovskaya Agar broth is mixed with 1,000 ml of distilled water and autoclaved in a L2 cycle for sterilization. 50 ml of sterilized broth is then poured into omni trays.

Preparation of bacteria. The bacteria of interest are inoculated in 24-well plates with 3 mL of TSB and grown for three days at room temperature. A positive control (a Pantoea ananatis strain known for its abilities to solubilize phosphorous) and negative control (E. coli DH5a) are grown in the same conditions. On the third day of growth, the bacteria are normalized to an initial OD₆₀₀ of 0.2 in 1×phosphate buffered saline buffer. These are further diluted 1:1,000 and 1:10,000 for screening.

Stamping the bacteria onto Pikovskaya agar. 2 μl of each dilution of bacteria is stamped onto 50 ml of Pikovskaya agar in omni trays in duplicate. All four plates are then placed in a 24° C. chamber for growth.

Imaging and Analyzing. Each plate is photographed. A jpeg image is taken and stored for analysis to be performed. The presence of “halos” is scored by eye. The presence of a halo is considered a positive result for that microbe. Microbes indicating positive results for more than 50% of replicates tested are considered capable of solubilizing phosphate.

### Example 24: High Throughput Screening Assay of Endophytes for Phytase Activity

This example describes a method for the detection and identification of bacterial microorganisms having the ability to solubilize phytic acid. In the present example, bacteria are stamped on phytate solubilizing media that allows for the bacteria to hydrolyze the phytic acid. When the bacteria hydrolyzes the phytic acid, a zone of clearing forms around that bacterial colony. Phytase activity is identified by a defined halo formation surrounding the bacterial colony.

Phytate solubilizing media preparation. To prepare 500 ml of phytate solubilizing media, the 2.5 g of Calcium phytate, 1.5 g of ammonium sulfate, 0.05 g of Calcium chloride, 0.05 Manganese sulphate pentahydrate, 0.05 Iron sulphate heptahydrate, 5 g glucose, and 7.5 g of agar are added to 350 mL of diH₂O. The solution pH is adjusted to 7.0, before bringing the total volume to 500 ml and finally adding the agar. After autoclaving, 50 ml of the media is poured into sterile omni trays and allowed to cool.

Bacterial inoculum preparation. The bacteria are inoculated in 3 ml of Tryptic Soy Broth (TSB) if vegetative, or sporulation media if spore forming. If vegetative, the bacteria are grown for 3 days before being normalized in phosphate buffer to an OD₆₀₀ of 0.2. If spore-forming, the bacteria is grown for 5 days before being normalized in phosphate buffer to an OD₆₀₀ of 0.2. Once normalized, the bacteria are diluted down to 10-7 in 96-well costar plates filled with 1×PBS.

After the microbes are normalized and diluted, 2 μl of the bacteria are stamped in triplicate onto the phytate solubilizing media omni trays at a concentration of 10⁻¹ using the Biomek FxP. These plates are allowed to incubate for 72 hours at 25° C. After incubation, the omni trays are imaged for the data collection. At this time, the presence of halos is measured.

Controls: Biological controls are used in each plate. The controls include a Pantoea ananatis strain known for its abilities to solubilize phytase for the positive control and E. coli DH5a as the negative control.

Data collection: When the omni trays with media are removed from the incubator, they are imaged under a standard, controlled light imaging station. The presence of “halos” is scored by eye. The presence of a halo is considered a positive result for that microbe. Microbes indicating positive results for more than 50% of replicates tested are considered to have phytase activity.

### Example 25: Determining the Protease Producing Capability of Endophyte Strains

The method of the following protocol enables the detection of bacteria able to break down extracellular protein through secretion of proteases. Proteases are enzymes which can hydrolyze peptide bonds and come in many forms such as cysteine, metallo, and serine proteases. The design of this assay does not determine what kind of protease is produced. The ability to produce a protease is inferred from the presence of a “halo” formation around the site of growth.

Skim Milk agar solution preparation. 51 grams of Skim Milk Agar broth is mixed with 1,000 mL of distilled water and autoclaved for sterilization. 50 ml of sterilized broth is then poured into omni trays.

Preparation of bacteria. The bacteria of interest are inoculated in 24-well plates with 3 mL of TSB and grown for three days at room temperature. The positive controls (B. thuringiensis and B. subtilis, both known for their protease production capabilities), and negative control (E. coli), are grown in the same conditions. On the third day of growth, the bacteria are normalized to an initial OD₆₀₀ of 0.2 in 1×phosphate buffered saline buffer. These are further diluted 1:1,000 and 1:10,000 for screening.

Stamping the bacteria onto Skim Milk agar. Two replicates of two dilutions are created by stamping 2 μl of each bacteria onto the omni tray. Plates are then placed in 24° C. chamber for growth.

Imaging and Analyzing. Each plate is photographed. A jpeg image is taken and stored for analysis to be performed. The presence of “halos” is scored by eye. The presence of a halo is considered a positive result for that microbe. Microbes indicating positive results for more than 50% of replicates tested are considered to have the ability to produce a protease.

### Example 26: Determining the Siderophore Production Capability of Endophyte Strains

The methods of the following example allow for the high throughput identification of a bacteria able to produce siderophores. In the present example, microbes are grown in an iron-deficient media to induce the expression of siderophore production gene pathways. This example adopts the CAS assay protocol and is scaled to a higher throughput plate-based format (H B. Schwyn and J. B. Neilands, Anal. Biochem. 160, 47 (1987). This method exploits the siderophore's high affinity for iron (III), which is initially bound in a chrome azurol S/ironIII/hexadecyltrimethylammonium bromide dye color indicator. When a strong chelator, such as a siderophore, removes iron from this complex, the color turns from blue to orange. The methods of this example employ spectrophotometric optical density metrics from 630-640 nm absorbance to measure the amount of ironIII lost from the CAS/FeIII/HDTMA complex.

Siderophore reagent solution preparation. All CAS assay solutions are prepared according to H B. Schwyn and J. B. Neilands, Anal. Biochem. 160, 47 (1987).

Experimental Methods. Microbes are prepared and inoculated at a normalized concentration of OD₆₀₀ into three replicate 96-well plates filled with 600 μl of Iron deficient King B media in each well. Microbes are grown for 3 days under agitation at 24° C. temperature, with gas exchange, in a controlled environment. The plates are then spun down in a centrifuge at 5000 rpm for five minutes. The Biomek is used to transfer 100 μl of microbial supernatant, 100 ul CAS assay solution, and 2 μl of shuttle solution to 96 well costar plate.

Controls: Biological and chemical controls are used in each experimental plate.

Data Collection: One method for data collection involves image capture under standard, controlled light conditions exactly 1 hour after combining reagents. Experimental 96 well plates are captured in both jpeg and CR2 formats. Images are then referenced for manual data capture. Any color change away from blue is recorded as a “hit”, meaning siderophore was produced by the given microbe. A second method of scoring employs spectrophotometric optical density metrics from 630-640 nm absorbance to measure the amount of ironIII lost from the CAS/FeIII/HDTMA complex. The following calculation, where Ar is the OD640 read of CAS/FeIII/HDTMA, and As is the OD640 read of the microbial supernatant plus CAS assay solutions, is then used to determine the approximate amount of siderophore units in solution. [(A r−As)/Ar]*100=% siderophore units.

### Example 27: Screening Endophytes for Potassium Solubilization Activity

Preparation of media. 29.605 g of Aleksandrow agar is suspended in 1000 ml of distilled water, then autoclaved. 50 ml of the solution is added to an omnitray and left to dry.

Culture Preparation. A sterile loop is used to inoculate a bacteria into a well of a 24-well plate. One well is reserved for the positive control (a Pantoea strain known for its nitrogen fixing capabilities), two wells are reserved for the negative controls (E. coli DH5a), and one well is left for un-inoculated media. The plate is then covered with a breathable membrane and incubated at room temperature in a shaker at 400 rpm for 3 days.

Bacterial Normalization. The cultures are diluted 1:2 in 1×PBS for OD reading and 1 mL of bacteria normalized to OD₆₀₀ in phosphate buffer.

Incubation and analysis. From the master plate a 1:10 serial dilution of the 96 bacteria is created. Two replicates of two dilutions (undiluted and 1:10) are created by stamping 2 μl of each bacteria onto TSA media omni tray. The plates are incubated for 7 days at 30° C. The presence of “halos” is scored by eye. The presence of a halo is considered a positive result for that microbe. Microbes indicating positive results for more than 50% of replicates tested are considered to have the ability to produce a solubilize potassium.

### Example 28: Screening Endophytes for Chitinase Producing Capacity

The methods of the following example enable a high throughput screen for determining the chitinase producing capability of bacterial and yeast strains.

Media Preparation: 1 L of Agar medium is combined with 22.2 ml/L of colloidal chitin. 50 mL media is poured into an omni tray and left to solidify.

Culture Preparation. A sterile loop is used to inoculate bacteria into a well of a 24-well plate. One well is reserved for the positive control (a Stenotrophomonas strain known for its chitinase producing capabilities), one well reserved for the negative controls (E. coli DH5a), and one well is left for un-inoculated media. The plate is then covered with a breathable membrane and incubated at room temperature in a shaker at 400 rpm for 3 days.

Bacterial Normalization. The cultures are then centrifuged, supernatant removed and re-suspended in 1×PBS solution, and diluted 1:2 for OD reading. The concentration of each bacteria is adjusted by diluting in PBS to 0.05 OD. 1:10 dilutions in 1×PBS are then created in a 96-well plate.

Media Inoculation, Incubation and colony scoring. 2 μl of each bacteria is stamped onto M9 agar in omni trays. The inoculated omni trays are parafilmed and inverted to prevent condensation from forming on the lid and are stored at room temperature for 72 hours.

The presence of “halos” zone clearing around the stamped bacteria is scored by eye. The presence of a halo is considered a positive result for that microbe. Microbes indicating positive results for more than 50% of replicates tested are considered to have chitinase activity.

Having illustrated and described the principles of the present invention, it should be apparent to persons skilled in the art that the invention can be modified in arrangement and detail without departing from such principles. It is to be understood that while the invention has been described in conjunction with the detailed description thereof, the foregoing description is intended to illustrate and not limit the scope of the invention, which is defined by the scope of the appended claims. Other embodiments, advantages, and modifications are within the scope of the following claims.

1. A method of improving plant health, comprising heterologously disposing one or more endophytes to a plant element in an effective amount to improve a trait of agronomic importance in a plant derived from the treated plant element relative to a reference plant derived from a reference plant element, wherein the one or more endophytes comprise a first endophyte comprising at least one polynucleotide sequence that is at least 97% identical to SEQ ID NO. 29-37, or 38.

2. The method of claim 1, wherein the one or more endophytes additionally comprises a second endophyte comprising at least one polynucleotide sequence that is at least 97% identical to SEQ ID NO. 39.

3. The method of claim 1, wherein the one or more endophytes comprise at least one sequence at least 97% identical to each of SEQ ID NO. 29-37, or 38.

4. The method of claim 1, wherein the one or more endophytes comprise at least one polynucleotide sequence at least 99% identical to SEQ ID NO. 29-37, or 38.

5. The method of claim 1, wherein the one or more endophytes comprise at least one polynucleotide sequence 100% identical to SEQ ID NO. 29-37, or 38.

6. The method of claim 1, wherein the one or more endophytes comprise at least one polynucleotide sequence at least 97% identical to each of SEQ ID NO. 29-37, or 38 and a second endophyte having at least one polynucleotide sequence at least 97% identical to SEQ ID NO. 39.

7. The method of claim 2, wherein the second endophyte comprises at least one polynucleotide sequence that is at least 99% identical to SEQ ID NO. 39.

8. The method of claim 2, wherein the second endophyte comprises at least one polynucleotide sequence that is 100% identical to SEQ ID NO. 39.

9. The method of claim 1, wherein the plant element is selected from the group consisting of wheat, rice, barley, buckwheat, rye, millet, oats, corn, sorghum, triticale and spelt.

10. The method of claim 1, wherein the plant element is selected from the group consisting of cotton, canola, sunflower, tomato, lettuce, peppers, cucumber, endive, melon, potato, and squash.

11. The method of claim 1, wherein the plant element is a whole plant, seedling, meristematic tissue, ground tissue, vascular tissue, dermal tissue, seed, leaf, root, shoot, stem, flower, fruit, stolon, bulb, tuber, corm, keikis, shoot, or bud.

12. The method of claim 1, wherein the one or more endophytes are heterologously disposed to a plant element via one or more seed treatments or soil pre-treatments, one or more foliar applications, and one or more floral applications.

13. The method of claim 1, wherein the trait of agronomic importance is selected from the group consisting of drought tolerance, heat tolerance, cold tolerance, salinity tolerance, metal tolerance, herbicide tolerance, improved water use efficiency, improved nitrogen utilization, improved nitrogen fixation, improved nutrient use efficiency, improved nutrient utilization, improved phosphorous use efficiency, biotic stress tolerance, yield improvement, health enhancement, vigor improvement, decreased necrosis, decreased chlorosis, decreased area of necrotic tissue, decreased area of chlorotic tissue, growth improvement, photosynthetic capability improvement, nutrition enhancement, altered protein content, altered oil content, increased biomass, increased shoot height, increased root length, increased shoot biomass, increased root biomass, increased leaf area, increased shoot area, increased root area, increased seedling area, improved root architecture, increased seed germination percentage, increased seed germination rate, increased seedling survival, increased survival, photosynthetic efficiency, transpiration rate, seed/fruit number or mass, plant grain or fruit yield, total grain weight, leaf chlorophyll content, photosynthetic rate, wilt recovery, turgor pressure, production of a volatile organic compound (VOC), increased seed weight, altered seed carbohydrate composition, altered seed oil composition, altered seed protein composition, altered seed nutrient composition, and combinations thereof.

14. The method of claim 9, wherein the plant element is wheat and the trait of agronomic importance is one of more of increased root length, increased yield, and increased shoot length.

15. The method of claim 9, wherein the plant element is corn and the trait of agronomic importance is one of more of total grain weight, nutrition enhancement, shoot fresh weight, increased yield, increased win rate, and improved grain fill.

16. The method of claim 9, wherein the plant element is rice and the trait of agronomic importance is one or more of increased shoot dry weight, and tiller number.

17. The method of claim 9, wherein the trait of agronomic importance is nutrition enhancement, and the nutrition enhancement comprises an increase in magnesium (Mg) or sulfur (S).

18. The method of claim 1, wherein an effective amount is an average application of least 1E+02 endophytes per seed, least 1E+03 endophytes per seed, at least 1E+04 CFU per seed, at least 1E+05 CFU per seed, at least 1E+06 CFU per seed, at least 1E+07 CFU per seed, or at least 1E+08 CFU per seed.

19. The method of claim 1, where the heterologously disposed one or more endophytes are formulated in a synthetic composition comprising one or more of a liquid state fermentation broth, a solid carrier, an adherent, talc, mineral oil, kaolin clay, a dispersant, and a surfactant, a sugar, a peptide, fertilizer, or a vitamin.

20. A synthetic composition, comprising one or more endophytes heterologously disposed to a treatment formulation, wherein the endophytes comprise one or more polynucleotide sequences at least 97% identical to one or more of SEQ ID NO. 29-38, wherein the endophyte in the synthetic combination is capable of improving a trait of agronomic importance in a plant or plant element compared to an untreated control plant.

21. The synthetic composition of claim 20, wherein the composition additionally comprises a plant element.

22. The synthetic composition of claim 20, wherein the one or more endophytes are capable of improving a trait of agronomic importance in a plant derived from the plant element relative to a plant derived from a reference plant element.

23. The synthetic composition of claim 20, wherein the plant element is selected from the group consisting of wheat, rice, barley, buckwheat, rye, millet, oats, corn, sorghum, triticale and spelt.

24. The synthetic composition of claim 20, wherein the plant element is selected from the group consisting of cotton, canola, sunflower, tomato, lettuce, peppers, cucumber, endive, melon, potato, and squash.

25. The synthetic composition of claim 20, wherein the trait of agronomic importance is selected from the group consisting of drought tolerance, heat tolerance, cold tolerance, salinity tolerance, metal tolerance, herbicide tolerance, improved water use efficiency, improved nitrogen utilization, improved nitrogen fixation, improved nutrient use efficiency, improved nutrient utilization, improved phosphorous use efficiency, biotic stress tolerance, yield improvement, health enhancement, vigor improvement, decreased necrosis, decreased chlorosis, decreased area of necrotic tissue, decreased area of chlorotic tissue, growth improvement, photosynthetic capability improvement, nutrition enhancement, altered protein content, altered oil content, increased biomass, increased shoot height, increased root length, increased shoot biomass, increased root biomass, increased leaf area, increased shoot area, increased root area, increased seedling area, improved root architecture, increased seed germination percentage, increased seed germination rate, increased seedling survival, increased survival, photosynthetic efficiency, transpiration rate, seed/fruit number or mass, plant grain or fruit yield, total grain weight, leaf chlorophyll content, photosynthetic rate, wilt recovery, turgor pressure, modulation of a metabolite, production of a volatile organic compound (VOC), modulation of the proteome, increased seed weight, altered seed carbohydrate composition, altered seed oil composition, altered seed protein composition, altered seed nutrient composition, and combinations thereof.

26. The synthetic composition of claim 20, additionally comprising a treatment formulation, wherein the treatment formulation comprises one or more of a surfactant, a buffer, a tackifier, a microbial stabilizer, a fungicide, an anticomplex agent, an herbicide, a nematicide, an insecticide, a plant growth regulator, a rodenticide, a desiccant, a nutrient, an excipient, a wetting agent, a salt, and a polymer.

27. The method of claim 20, wherein the treatment formulation comprises one or more of a liquid state fermentation broth, a solid carrier, an adherent, talc, mineral oil, kaolin clay, a dispersant, and a surfactant, a sugar, a peptide, fertilizer, or a vitamin.

28. The method of claim 20, wherein the synthetic composition additionally comprises one or more endophyte of the genus Bacillus.

29. The method of claim 20, wherein the synthetic composition additionally comprises one or more endophyte of the genus species Bacillus simplex.

30. The synthetic composition of claim 20, wherein the one or more endophytes comprise at least one polynucleotide sequence at least 97% identical to each of SEQ ID NOs. 29-38.

31. A synthetic composition, comprising one or more endophytes heterologously disposed to a plant element, wherein the one or more endophytes comprises a first endophyte having at least one polynucleotide sequence that is at least 97% identical to SEQ ID NO. 29-37, or 38, and a second endophyte having at least one polynucleotide sequence that is at least 97% identical to SEQ ID NO. 39.

32. The synthetic composition of claim 31, wherein the synthetic composition additionally comprises 1) a treatment formulation, wherein the treatment formulation comprises one or more of a surfactant, a buffer, a tackifier, a microbial stabilizer, a fungicide, an anticomplex agent, an herbicide, a nematicide, an insecticide, a plant growth regulator, a rodenticide, a desiccant, a nutrient, an excipient, a wetting agent, a salt, and a polymer, and or 2) a plant element, wherein the plant element is a seed.

33. The synthetic composition of claim 32, wherein the seed is a cereal seed.


SEQUENCE LISTING (39 sequences)

SEQ ID NO: 1 - DNA, 19 bp, synthetic construct
  source 1..19: mol_type = other DNA
ghgacaagcgtttctcngg

SEQ ID NO: 2 - DNA, 21 bp, synthetic construct
  source 1..21: mol_type = other DNA
cttvavytggaacttgatggt

SEQ ID NO: 3 - DNA, 19 bp, synthetic construct
  source 1..19: mol_type = other DNA
atgtgcaaggccggtttcg

SEQ ID NO: 4 - DNA, 20 bp, synthetic construct
  source 1..20: mol_type = other DNA
tacgagtccttctggcccat

SEQ ID NO: 5 - DNA, 20 bp, synthetic construct
  source 1..20: mol_type = other DNA
ccgagtacaaggargccttc

SEQ ID NO: 6 - DNA, 21 bp, synthetic construct
  source 1..21: mol_type = other DNA
ccgatrgaggtcatracgtgg

SEQ ID NO: 7 - DNA, 20 bp, synthetic construct
  source 1..20: mol_type = other DNA
taagtagccccgagactgct

SEQ ID NO: 8 - DNA, 20 bp, synthetic construct
  source 1..20: mol_type = other DNA
cttctgggtggcagtgtagg

SEQ ID NO: 9 - DNA, 22 bp, synthetic construct
  source 1..22: mol_type = other DNA
cttggtcatttagaggaagtaa

SEQ ID NO: 10 - DNA, 17 bp, synthetic construct
  source 1..17: mol_type = other DNA
tcctgagggaaacttcg

SEQ ID NO: 11 - DNA, 20 bp, synthetic construct
  source 1..20: mol_type = other DNA
gctgcgttcttcatcgatgc

SEQ ID NO: 12 - DNA, 20 bp, synthetic construct
  source 1..20: mol_type = other DNA
gcatcgatgaagaacgcagc

SEQ ID NO: 13 - DNA, 19 bp, synthetic construct
  source 1..19: mol_type = other DNA
gtgartcatcgaatctttg

SEQ ID NO: 14 - DNA, 20 bp, synthetic construct
  source 1..20: mol_type = other DNA
tcctccgcttattgatatgc

SEQ ID NO: 15 - DNA, 17 bp, synthetic construct
  source 1..17: mol_type = other DNA
gtygayttcaaygtycc

SEQ ID NO: 16 - DNA, 20 bp, synthetic construct
  source 1..20: mol_type = other DNA
acaccdggdggrccgttcca

SEQ ID NO: 17 - DNA, 20 bp, synthetic construct
  source 1..20: mol_type = other DNA
gartgyccdggdcayttygg

SEQ ID NO: 18 - DNA, 23 bp, synthetic construct
  source 1..23: mol_type = other DNA
ccngcdatntcrttrtccatrta

SEQ ID NO: 19 - DNA, 23 bp, synthetic construct
  source 1..23: mol_type = other DNA
cccatrgcytgyttmcccatdgc

SEQ ID NO: 20 - DNA, 20 bp, synthetic construct
  source 1..20: mol_type = other DNA
gaygaycgkgaycayttcgg

SEQ ID NO: 21 - DNA, 20 bp, synthetic construct
  source 1..20: mol_type = other DNA
cccatrgcytgyttrcccat

SEQ ID NO: 22 - DNA, 23 bp, synthetic construct
  source 1..23: mol_type = other DNA
cccatrgcytgyttmcccatdgc

SEQ ID NO: 23 - DNA, 20 bp, synthetic construct
  source 1..20: mol_type = other DNA
gaygaymgwgatcayttygg

SEQ ID NO: 24 - DNA, 20 bp, synthetic construct
  source 1..20: mol_type = other DNA
cccatwgcytgcttmcccat

SEQ ID NO: 25 - DNA, 20 bp, synthetic construct
  source 1..20: mol_type = other DNA
taccatcgagaagttcgaga

SEQ ID NO: 26 - DNA, 20 bp, synthetic construct
  source 1..20: mol_type = other DNA
catccttggagataccagcc

SEQ ID NO: 27 - DNA, 23 bp, synthetic construct
  source 1..23: mol_type = other DNA
gtbcacctycaraccggycartg

SEQ ID NO: 28 - DNA, 26 bp, synthetic construct
  source 1..26: mol_type = other DNA
ccrgaytgrccraaracraagttgtc

SEQ ID NO: 29 - DNA, 485 bp, Coniochaeta nivea
  source 1..485: mol_type = genomic DNA
caagaagccgaaaggctacttcaaaccattgcgaactcgtccaagttgcttcggcggcgcggcaccccctaacgggggcgccgcagccctgcctctccggaggttcggggcgcccgccggaggtacgaaactctgtattatagtggcatctctgagtataaagcaaataagttaaaactttcaacaacggatctcttggctctggcatcgatgaagaacgcagcgaaatgcgataagtaatgtgaattgcagaattcagtgaatcatcgaatctttgaacgcacattgcgcccggtagtactctaccgggcatgcctgttcgagcgtcatttcaaccctcaagctctgcttggtgttggggccctacggctgccgtaggccctgaaaggaagtggcgggctcgctacaactccgagcgtagtaattcattatctcgctagggaggttgcggcgtgctcctgccgttaaagacccatctttaacca

SEQ ID NO: 30 - DNA, 407 bp, Coniochaeta nivea
  source 1..407: mol_type = genomic DNA
cttcagctggaacttgatggtcgacttgacctcggtgatcttgttggagaggtcatcggcgtgggacacgggggtcgggaacttgccggccttggagagaccgggaccgagcagacgggggatctgcttgatcagggtgtcggaggcgatgaaagcggcgtacttgcgggccagcttcttgatcactgttgtatcggttagtatcgaggtcaaagaccaggaaggcgaaccggttgccacatacgcttcttgttcttgttcagcttcttcaagtcgtcggccgacatggagtcgacaccgccgtgcttcgcacgatcgatatcgtgctggtcaccgagaatgcagatggccatgttcgggcggggaacagtgggcagcttgacggtgccagagaaacgcttgtcacg

SEQ ID NO: 31 - DNA, 549 bp, Coniochaeta nivea
  source 1..549: mol_type = genomic DNA
ccggtatcgtgctcgactccggtgacggtgtcacccacgttgtgcccatctacgagggtttcgctcttccccacgccattgcccgtgtcgacatggctggccgtgacttgaccgactacctcatgaagatccttgctgagcgcggttacgccttctccaccactgccgagcgcgaaatcgttcgtgacatcaaggagaagctctgctacgttgccctcgacttcgagcaggagatccagaccgccgcccagagctccagcttggagaagtcgtatgagcttcccgacggtcaggtcatcaccctcggcaacgagcgcttccgtgctcctgaggctctcttccagccttctgtcctcggtctcgagagcggtggcatccacgtcaccactttcaactcgatcatgaagtgcgacgtcgatgtccggaaggatctctatggtaacatcgttatggtacgttctgcctcttatggctcctcaaagcgaaaacaatggaagaatatagctaacagcgcactagtctggtggcaccaccatgtaccccggtctc

SEQ ID NO: 32 - DNA, 542 bp, Coniochaeta nivea
  source 1..542: mol_type = genomic DNA
ggtcgaaaacctgttgatatcggttagcacgtgtgctatatctgttataaaaaatgtgattgtatgtatcttgccttgaaagcttctcgaatctcttcctcggaatcagtatccttcatcttccgggccatcatggtaagaaactctggcaatcgtcagcgccgcgacctccacacgtgtacttttcaagtgatagacctaccggggaagtcaatggtgccgttgttgtcggcatcaacctcgttgatcatgtcttggagctcggactcggaggggttctgtcctagagagcgcatgaccgttcccagttccttggtagtgatttgtccttggtgtaagtcagcaatgttgttactcgagaaatgagatcgagtggtaaggcagtagcagcgacacagcgcctgcggatctatcgacgccgtagaattgatcgcgcatccgcatttgccgcagagagataaaggtagggcagtactaaccatcgccatctttgtcctgaacatgcgcccgtcagcaatagtcacacgcgccgaacaagacgc

SEQ ID NO: 33 - DNA, 521 bp, Coniochaeta nivea
  source 1..521: mol_type = genomic DNA
aacgaggagacctacgacggcaaggccgacgtcatctccaacgcctcgtgcaccaccaactgccttgcccccctcgccaaggtcgtcaacgacaagttcaccatcattgagggtctgatgaccaccatccactcctacaccgccacccagaagaccgtcgacggtccttcgtccaaggactggcgcggtggccgtggtgctgcccagaacatcatccccagcagcaccggtgccgccaaggccgtcggcaaggtcatccccgagctcaacggcaagctgaccggcatgtcgatgcgtgtccccacctccaacgtctccgttgtcgatcttacctgccgtatcgagaagcctgcttcgtacgacgagatcaaggccgccatcaaggaggccgctgagggtcccctcaagggtatgtctcattctcctcaacccctaaatgaaaccagaacagaaaagctaacctgtcccaggagtccttgcctacaccgaggacgaggtcgtctcctccgacatgaacggca

SEQ ID NO: 34 - DNA, 1022 bp, Coniochaeta nivea
  source 1..1022: mol_type = genomic DNA
acaccggcggggccgttccacaggatggtctgcgcgtcgccgatggcctcctcgtacagcttgatcgactccgggccgcagtcgaggcccatccagccgtcggggatgccgtccttgtccgtggcggtgccagtctgcgcgtccttgtcgaacttgtcggcgataacgtagtcgacggggagcacgaccttgacgttgttggccttggccttctccatgagcttgccgaccgtcttggcgccggcctcgtcgaagagcgaggtgccgatggggatgttgtccagcgtcttcttgaaggtgaaggccatgccgccgcagatgacgagcgtgtccaccttctcgagcaggttgtcgatcagctggatcttgtcgctgaccttggcgccgcccaggatggccaggaagggccgcttggggttctcgagggccttggcgaagtagtcgagctccttcttcatcaggtagccggcggccttttgtgggaggtcgatgccgaccatggaggagtgggcgcggtgtgcggtgccgaaggcatcgtctgaagtgcggttgtgttagtttcaggggcgcatactagagctgctgtctgacaaaacttactgacgtaaacgtccccgagcttggtgagtcccttgcggaactcctcgaccttggccttgtcggccttgaccttgttgccctcggcatcaacgcccttgccctcctcctcgatgtggaagcggaggttctccagcagcacaacgtcgccattgtccgccttggagacgatctcctcgacctcggggccgacgcagtccggcgcgaaggtgaccttcttgcccagcagcttctcgagctcgggcacgacgggcttgagcgagaacttctccttgacctggccgtcggggcggccgaggtgggacatgaggatcacggccttggcgccgttgtcgatggcatacttgattgtggggatggcgccggcaatgcgctggttgttggtgaccttcttgttcgcgtcgagggggacgttgaagtcaacc

SEQ ID NO: 35 - DNA, 810 bp, Coniochaeta nivea
  source 1..810: mol_type = genomic DNA
ccagcaatatcgttgtccatgtacgtggcaacgtgatactgcagcagatcctcgaaatccgtgataatgtgctgaggcgcaccatccgcgatagcttgcctaacattgctgttggctcgaatgatgtcaccaagcttgtatgtcaaatcgtcctcgttacgcaggcctgtcccgctgccgtccatggaaatgctaggccgaacaggaggtggcggcacgggaagaacggtgacaatcaaccactctgggcgagcatagtcaacgttgaggcccatatcacgaaggtccgaatccgagatccggcgaagaatgccgtgagccatctctggtgtcaaaaccgttttgtctttgacggtagcaccatcctcggtcttcgagctcttcgtcgtaaacagctgaagagctgcctggcgaaccgtcggctgctggtttccacatccaccatgcttgattgtcggcttagcattcggatcattggaaccaaattcggcatccttgtcctgcttgacatccttctctatgtagcacatggtctgcttcttgcaaacagcccacacgcggttgaagcgaagtttggggtctctgatattgacggcggcgagaaattcatcatcagactagagcggagtgcaagattagcatcaagaagatcatgcaaagcaaccaaagcagtgcagactaaccctgtcagccaacaccttgctacagttatgacagacgatctcgaggatcttcttgactttcttgatgaagccagggtggtagacgggtttagccaactcgatgtgaccaaaatggcccggacactca

SEQ ID NO: 36 - DNA, 1167 bp, Coniochaeta nivea
  source 1..1167: mol_type = genomic DNA
cccatggcttgcttgcccatggcagactgataggtgtttcggggcgactggttgtggtcagggaacgggataatgctggcgcagatgcccaggagcatgctggggtggatttcgcagtgagtgtaggcgtgtgtggtgggatttatccgggtcttgatgcgcgcgttgggctcattctcgtggttgtccggaatcttgaagccagccttttgtgctcggaagtcggcaagatcttccggcgtcatacatatcatggatgtctcttcctcctcagcgtcgaggtagtcgatgcagcctgcgtcctccaaccccttgaagccaaagtactctgggtcgtccttggctaattgagcgtcgcgtctgagcttctcaatgtgcgtctggttgatgaccagctcgcccacaggtgtttccacgttctcccgtgtctcaacgacgaacagcggtctcatcacacgtcccgcatcggagaagatcttgaattctcggtctcgaatatctcggaccagcgagacctcgggcggtatgtcaccctcacgacggaggtttgtgatttgagccacaagattcttcggatcctggtgcacgccgacccatgtgccgttgacaaaaaccttggttgcgttcgggaaccgaaggggctcgtactcctccaacacctccatattccttgcaatcatgaagtcgataagcggctcagcgggttgccccacgctcacgtagcacatgagggccagattcttgacaaggccacaagcttgcccctcgggggtctcagcagggcagacaagaccccaatgtgtgttgtgcagttgtcgtggctttgctaacttgccgtcacggccaattggcgtgttggtgcgtctcaaatgcgacagtgtggaagcgaacgtataacggttcagcacttgcgagacaccagcggtggtgctcattgccttcttctggtcgccccagtttcctgtcgcaagagagtacttcagagcgttggtgatggtggacgggtggatgccctggaccaacgagaacttcttgtcttggtccacgcacttcttcaagtagctcatgaggtcctgcgtcattctgcgaacgatgtttcgaaaaagcttcgccagcagaggaccggccaggtcgaggcgcttcttgccaaagtggtcacggtcgtcg

SEQ ID NO: 37 - DNA, 871 bp, Coniochaeta nivea
  source 1..871: mol_type = genomic DNA
ctcatcattgccgctggtactggtgagttcgaggctggtatctccaaggatggccagactcgtgagcacgctctgctcgccttcaccctcggtgtcaggcagctcatcgtcgccatcaacaagatggacaccgccaagtggtctgaggatcgttacaacgagatcatcaaggagacctcttccttcatcaagaaggtcggctacaaccccaagcaggtcgctttcgtccccatctcgggtttcaacggcgacaacatgctggccgcctccaccaactgcccctggtacaagggctggaagaaggagggcaagaacggtgagatcaccggcaagaccctcctcgaggccatcgactcttgcgagccccccaagcgtcctaccgacaagcccctccgtcttcccctccaggatgtttacaagatcggcggcattggaacagtgcccgtcggccgtatcgagactggaaagctcattcccggtatggtcgttaccttcgctcccgccaacgtcaccactgaagtcaagtccgtcgagatgcaccacgagcagctcaaggagggtctccccggtgacaacgtcggcttcaacgtgaagaacgtctccgtcaaggagatccgtcgtggcaacgtcgctggtgacagcaagaacgacccccccatgggcgccgcttctttcctcgcccaggtcatcgtcctgaaccaccccggtcaggtcggtgctggatacgccccggttctcgactgccacactgcccacattgcctgcaagttcgccgagatccaggagaagattgaccgccgtaccggaaagtcgactgagtccaaccccaagttcatcaagtcgggtgacgctgccatcgtcaagatgattc

SEQ ID NO: 38 - DNA, 529 bp, Coniochaeta nivea
  source 1..529: mol_type = genomic DNA
cccgactggccgaaaacgaagttgtcgggacggaagagctgaccgaagggaccagcgcggacagcgtccatggtacccggttcgagatcgacgaggacagcgcggggaacatacttgttgccggaagcctagttttgtgattacgagtcaatagctgcggtatggagccatggcttgatgactggatggatagcttacctcgttgaagtagacgctcatacgctcgagctggagctccgaagtgccgttgtacctagatggcggtgagcttgagcggtacgaagatcacgttagagctgttacgtacacgccattgctgtcgaggccgtgctcgccggagatagtttgcctgttatcgttattagaatctcagttcgaacgcaggtctcaacgtcgagttgttggtctgaaggcgaacgcaccagaaggcagcaccgatttggttaccctgcgagacatgttagcaggccatcactcaagcacgagaagatagacacgacttacgcattgaccggtctgaagatggacc

SEQ ID NO: 39 - DNA, 1479 bp, Bacillus simplex
  source 1..1479: mol_type = genomic DNA
ggctcaggacgaacgctggcggcgtgcctaatacatgcaagtcgagcgaatcgatgggagcttgctccctgagattagcggcggacgggtgagtaacacgtgggcaacctgcctataagactgggataacttcgggaaaccggagctaataccggatacgttcttttctcgcatgagagaagatggaaagacggtttacgctgtcacttatagatgggcccgcggcgcattagctagttggtgaggtaatggctcaccaaggcgacgatgcgtagccgacctgagagggtgatcggccacactgggactgagacacggcccagactcctacgggaggcagcagtagggaatcttccgcaatggacgaaagtctgacggagcaacgccgcgtgaacgaagaaggccttcgggtcgtaaagttctgttgttagggaagaacaagtaccagagtaactgctggtaccttgacggtacctaaccagaaagccacggctaactacgtgccagcagccgcggtaatacgtaggtggcaagcgttgtccggaattattgggcgtaaagcgcgcgcaggtggttccttaagtctgatgtgaaagcccacggctcaaccgtggagggtcattggaaactggggaacttgagtgcagaagaggaaagtggaattccaagtgtagcggtgaaatgcgtagagatttggaggaacaccagtggcgaaggcgactttctggtctgtaactgacactgaggcgcgaaagcgtggggagcaaacaggattagataccctggtagtccacgccgtaaacgatgagtgctaagtgttagagggtttccgccctttagtgctgcagctaacgcattaagcactccgcctggggagtacggccgcaaggctgaaactcaaaggaattgacgggggcccgcacaagcggtggagcatgtggtttaattcgaagcaacgcgaagaaccttaccaggtcttgacatcctctgacaaccctagagatagggctttccccttcgggggacagagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatggatggtacaaagggctgcaaacctgcgaaggtaagcgaatcccataaagccattctcagttcggattgcaggctgcaactcgcctgcatgaagccggaatcgctagtaatcgcggatcagcatgccgcggtgaatacgttcccgggccttgtacacaccgcccgtcacaccacgagagtttgtaacacccgaagtcggtgaggtaaccttcatggagccagccgcctaaggtgggacagatgattggggt